(19) United States
(12) Patent Application Publication
(10) Pub. No.: US 2015/0105696 A1
(43) Pub. Date: Apr. 16, 2015

(54) Instruments, Methods and Systems for Harvesting and Implanting Graft Materials

(71) Applicant: Accelerated Orthopedic Technologies, Inc., Guildford, CT (US)

(72) Inventors: Ronald G. Litke, Sandy Hook, CT (US); Ernest Corrao, Bethel, CT (US)

(73) Assignee: Accelerated Orthopedic Technologies, Inc., Guilford, CT (US)

(21) Appl. No.: 14/401,589

(22) PCT Filed: May 23, 2013
(86) PCT No.: PCT/US13/42414

§ 371 Date: Nov. 17, 2014

Related U.S. Application Data
(60) Provisional application No. 61/788,693, filed on Mar. 15, 2013.
(60) Provisional application No. 61/664,976, filed on Jun. 27, 2012.
(60) Provisional application No. 61/650,841, filed on May 23, 2012.

(51) Int. Cl. A61F 2/30; A61B 17/16; A61F 2/46; A61B 5/107; A61B 5/00

(52) U.S. Cl. CPC A61F 2/30756; A61B 5/1077; A61B 5/742; A61F 2/4657; A61B 17/1635; A61B 5/4836; A61F 2002/4663; A61F 2002/30764
USPC 600/587; 606/102; 606/79

(57) ABSTRACT

Exemplary embodiments are directed to instruments, methods and systems for harvesting and implanting graft materials, including instruments for capturing a surface topography of an anatomical location, instruments for defining an implant region, and graft harvesting devices. Exemplary embodiments are also directed to methods for capturing a surface topography of an anatomical location, methods for defining an implant region, and methods for harvesting a donor plug. The exemplary instruments, methods and systems generally include capturing a surface topography of a defect region, creating a defect region cavity, and harvesting a donor plug configured and dimensioned to be implanted in the defect region cavity.

### CROSS-REFERENCE TO RELATED APPLICATIONS

This application claims the benefit of co-pending, commonly assigned U.S. Provisional Patent Application No. 61/650,841 entitled “Instruments, Methods and Systems for Defining Implant Site For Cartilage Materials,” filed on May 23, 2012, U.S. Provisional Application No. 61/664,976 entitled “Instruments, Methods and Systems for Defining Implant Site For Cartilage Materials,” filed on Jun. 27, 2012, and U.S. Provisional Application No. 61/788,693 entitled “Instruments, Methods and Systems for Harvesting and Implanting Graft Materials,” filed on Mar. 15, 2013. The entire content of the foregoing provisional patent applications is incorporated herein by reference.

### TECHNICAL FIELD

The present disclosure relates to instruments, methods and systems for use in defining an implant sites for graft materials, e.g., cartilage, and harvesting graft materials from donor sites. More particularly, the present disclosure provides apparatus and systems that may be used by clinicians to define implant sites for osteochondral grafts of desired shapes, sizes and/or depths, to acquire osteochondral grafts of desired shapes, sizes and/or depths in an efficient and reliable manner, and to implant such grafts in desired locations. The disclosed instruments, methods and systems have wide clinical utility and applicability, and may be employed with beneficial results to harvest and/or implant allograft, autograft and/or synthetic materials.

### BACKGROUND

Articular cartilage is a complex structure that, once damaged, has little capacity for permanent repair. One technique that has received attention for addressing cartilage-related issues involves repair with living hyaline cartilage through osteochondral autograft transplant. The procedure is known as mosaicplasty and generally involves removing injured tissue from a damaged area. One or more cylindrical sockets are drilled into the underlying bone and a cylindrical plug graft—consisting of healthy cartilage from the knee—is implanted in each socket.

As discussed in PCT applications entitled “Systems, Devices and Methods for Cartilage and Bone Grafting” and “Instruments, Methods and Systems for Harvesting and Implanting Cartilage Materials,” which published as WO 2009/154691 A9 (corrected version) and WO 2011/008968 A1, respectively, commercially available instruments for use in mosaicplasty procedures include Acufex instruments available from Smith & Nephew, Inc. (Andover, Mass.), the COR System available from Innovasive Technologies (Marlborough, Mass.), and the Arthrex Osteochondral Autograft Transfer System available from Arthrex (Naples, Fla.). The contents of the foregoing PCT applications are incorporated herein by reference.

Despite efforts to date, a need remains for instruments and systems for efficient, effective and reliable access to desired graft/cartilage sites and removal of desired graft/cartilage tissue. In addition, a need remains for instruments/systems that facilitate graft/cartilage access and/or removal in a minimally less invasive manner. Still further, a need remains for instruments/systems that facilitate effective, efficient and reliable selection of donor graft/cartilage sites and/or graft/cartilage source materials that geometrically match the removed cartilage tissue and/or void region. These and other needs are met by the instruments/systems and associated methods disclosed herein.

### SUMMARY

In accordance with embodiments of the present disclosure, an instrument for capturing a surface topography of an anatomical location is provided, generally including a plurality of elongated rod members and a locking mechanism. The locking mechanism generally releasably secures the plurality of elongated rod members relative to each other. The plurality of elongated rod members can be oriented to capture the surface topography of the anatomical location. The plurality of elongated rod members can independently translate relative to each other. The surface topography of the anatomical location generally includes a combination of a peripheral surface topography and a central surface topography of the anatomical location of a defect region. In some embodiments, the plurality of elongated rod members can include a visual indicator, e.g., a color variation, a texture variation, and the like, thereon to indicate an effectiveness of the plurality of elongated rod members to capture the surface topography of the anatomical location.

In accordance with embodiments of the present disclosure, an instrument for defining an implant region is provided, generally including a template and an adapter. The template can be configured to be driven into the anatomical location and/or secured to the anatomical location with, e.g., screws, K-wires, and the like. The adapter generally includes a plurality of elongated rod members for capturing a surface topography surrounding a defect region. In some embodiments, the plurality of elongated rod members can include a visual indicator, e.g., a color variation, a texture variation, and the like, thereon to indicate an effectiveness of the plurality of elongated rod members to capture the surface topography surrounding the defect region. The template generally defines a geometry configured and dimensioned to capture or substantially surround the defect region in the anatomical location. The geometry of the template can be one of a predetermined geometry or a variable geometry. For example, in some embodiments, the template geometry can be variable such that a user can adjust the geometry based on the configuration or dimensions of the defect region. The geometry of the template can be one of an asymmetrical geometry or a symmetrical geometry.

In some embodiments, the adapter can be detachable from the template. The exemplary instrument includes a driving mechanism, e.g., a hammer mechanism, a crank-actuated mechanism, combinations thereof, and the like, and a cutter. The hammer mechanism can be configured as a slap hammer or can facilitate the use of a drill. The crank-actuated mechanism generally includes a screw for anchoring the template to the defect region and an actuator for driving the template into the anatomical location. The driving mechanism generally drives the template into the anatomical location. As the driving mechanism drives the template into the anatomical location, the adapter generally captures the surface topography surrounding the defect region. In some embodiments, rather than driving the template into the anatomical location, screws, K-wires, and the like, can be used to secure the template to the anatomical location prior to forming a defect region cavity. The cutter generally forms one of a smooth defect region cavity or a stepped defect region cavity. The stepped defect region cavity can create a press fit between the stepped defect region cavity and a donor plug.

In accordance with embodiments of the present disclosure, an exemplary instrument for defining an implant region is provided, generally including a punch and a guide. The guide can be detachably engaged relative to the punch. In general, the guide includes a mounting mechanism, e.g., an internally threaded aperture, configured and dimensioned to facilitate interaction with an ancillary device, e.g., a hammer mechanism, a crank-actuated mechanism, a drill, and the like. The punch and the guide can cooperatively define a mechanism, e.g., a slide mechanism, a keying feature, combinations thereof, and the like, for detachably mounting the guide relative to the punch.

The guide includes an aperture configured and dimensioned to permit cutting passage therethrough. In some embodiments, the instrument includes a cutter member guide detachably engaged relative to the punch configured and dimensioned to permit cutter passage therethrough. Repositioning of the guide or the cutter member guide relative to the punch generally facilitates cutting action at a distinct location relative to the punch.

The punch can define a peripheral cutting edge, e.g., a clean cutting edge, a serrated cutting edge, and the like. In some embodiments, rather than or in combination with being driven into an anatomical location with the peripheral cutting edge, the punch can be secured or fixated to the anatomical location with, e.g., screws, K-wires, and the like, without using a hammering mechanism or mallet. In some embodiments, the punch can be partially driven into the anatomical location to position the punch relative to the anatomical location and alternative devices, e.g., screws, K-wires, and the like, can be used to secure the punch to the anatomical location. The punch generally includes at least one inner wall that defines an interior cutting region within the punch, e.g., an asymmetrical geometry, a symmetrical geometry, and the like. In some embodiments, the punch includes a peripheral template track configured and dimensioned to partially receive a mounting track therein. The punch further includes a plurality of peripheral apertures configured and dimensioned to receive therein a locking screw for securing the mounting track to the punch. The mounting track includes a plurality of apertures configured and dimensioned to receive therethrough K-wires for securing the mounting track to an anatomical location. In some embodiments, the punch includes a peripheral protrusion. Alignment of the peripheral relative to a bushing of a cutter can visually indicate a position of a drill bit within the punch.

In accordance with embodiments of the present disclosure, an exemplary graft harvesting device is provided, generally including an elongated shaft and a cutting member. The cutting member can be mounted with respect to the elongated shaft and can be operative to form a harvest cavity of a predetermined geometry. The exemplary device generally includes a plurality of elongated rod members for capturing a peripheral surface topography of an anatomical location surrounding the defect region cavity. In general, the device includes a locking mechanism for releasably locking the plurality of elongated rod members in a desired relative orientation. The plurality of elongated rod members can be actuated manually and/or electronically to translate against a surface of the anatomical location. In some embodiments, the plurality of elongated rod members include a visual indicator, e.g., a color variation, a texture variation, and the like, thereon to indicate an effectiveness of the plurality of elongated rod members to capture the peripheral surface topography of the anatomical location surrounding the defect region cavity.

The exemplary device can include a driving mechanism, e.g., a hammer mechanism, a crank-actuated mechanism, a spring-actuated mechanism, combinations thereof, and the like. The crank-actuated mechanism includes a platform for securing a donor cartilage thereon. The platform can be translatable relative to the cutting member. The crank-actuated mechanism generally includes an actuator for driving the donor cartilage into the cutting member and/or driving the cutting member into the donor cartilage. The predetermined geometry of the harvest cavity can be asymmetrical or symmetrical. The device generally includes a broach movably mounted within the cutting member. The broach can be axially translatable into a protruded position extending out of the cutting member and a retracted position within a cavity of the cutting member. The device generally includes a broach flange for regulating a position of the broach between the protruded position and the retracted position. In some embodiments, the device includes a cutter guide for trimming a donor plug extending from the cutting member.

In accordance with embodiments of the present disclosure, an exemplary method for capturing a surface topography of an anatomical location is provided, generally including establishing a referential orientation of an instrument relative to the anatomical location. The instrument generally includes a plurality of elongated rod members and a locking mechanism for releasably securing the plurality of elongated rod members relative to each other. The plurality of elongated rod members can be orientated to capture the surface topography of the anatomical location. The exemplary method includes pressing the plurality of elongated rod members against the anatomical location to capture the surface topography of the anatomical location. Pressing the plurality of elongated rod members against the anatomical location generally independently translates each of the plurality of elongated rod members relative to each other.

In accordance with embodiments of the present disclosure, an exemplary method for defining an implant region is provided, generally including establishing a referential orientation of an instrument relative to an anatomical location. The instrument generally includes a template configured to be driven into and/or secured the anatomical location and an adapter including a plurality of elongated rod members for capturing a surface topography surrounding a defect region. The exemplary method includes driving the template into and/or securing the template to the anatomical location. For example, in some embodiments, the template can be driven into the anatomical location. In some embodiments, the template can be partially driven into the anatomical location and can be further secured or fixated to the anatomical location with, e.g., screws, K-wires, and the like. In some embodiments, rather than driving the template into the anatomical location, screws, K-wires, and the like, can be used to secure the template to the anatomical location. Driving the template into and/or securing the template to the anatomical location simultaneously captures the surface topography surrounding the defect region by pressing the plurality of elongated rod members against the anatomical location. The method generally includes forming one of a smooth defect region cavity or a stepped defect region cavity with a cutter. The method includes press fitting a donor plug into the stepped defect region cavity.

In accordance with embodiments of the present disclosure, an exemplary method for defining an implant region is provided, generally including establishing a referential orientation of an instrument relative to an anatomical location. The instrument generally includes a punch and a guide detachably engaged relative to the punch. The guide includes a mounting mechanism configured and dimensioned to facilitate interaction with an ancillary device. The exemplary method includes driving the punch into and/or securing the punch to the anatomical location. For example, in some embodiments, the punch can be driven into the anatomical location. In some embodiments, the punch can be partially driven into the anatomical location and can be further secured or fixated to the anatomical location with, e.g., screws, K-wires, and the like. In some embodiments, rather than driving the punch into the anatomical location, screws, K-wires, and the like, can be used to secure the punch to the anatomical location.

In general, the method includes introducing a cutter through an aperture in the guide to define a first cut in the anatomical location. The method further includes repositioning the guide relative to the punch to reposition the aperture relative to the punch and reintroducing the cutter through the aperture in the guide to define a second cut in the anatomical location. The method includes removing the guide from the punch and introducing a clean-up cutter to an internal region defined by the punch. In some embodiments, the method includes detaching the guide from the punch and detachably engaging a cutter member guide to the punch. The cutter member guide includes an aperture configured and dimensioned to permit cutter passage therethrough and can be repositioned relative to the punch to reposition the aperture for creating a first and second cut. In some embodiments, the method includes securing a mounting track to a peripheral template track in the punch.

In accordance with embodiments of the present disclosure, an exemplary method for harvesting a donor plug is provided, generally including establishing a referential orientation of a graft harvesting device relative to a donor cartilage. The graft harvesting device generally includes an elongated shaft and a cutting member mounted with respect to the elongated shaft. The cutting member can be operative to form a harvest cavity of a predetermined geometry. The method generally includes driving the cutting member into the donor cartilage. The exemplary device generally includes a broach and a plurality of elongated rod members. The method includes axially translating the broach within the cutting member into a protruded position extending out of the cutting member. In general, the method includes driving the broach into a defect region cavity and capturing a surface topography surrounding the defect region cavity with the plurality of elongated rod members. The exemplary method includes matching the captured surface topography to a complementary topography of the donor cartilage. In some embodiments, the method includes trimming the excess portion of the donor plug extending from the cutting member.

In accordance with embodiments of the present disclosure, an exemplary instrument is provided for capturing a surface topography of a defect region. In particular, the exemplary topographical instrument generally includes a plurality of movably mounted elongated rod members and a locking mechanism for releasably securing the plurality of elongated rod members relative to each other and relative to the instrument axis. The plurality of elongated rod members may be advantageously configured and/or oriented to capture an entire surface topography of the anatomical location of a defect region, including a combination of a peripheral surface topography and a central surface topography. Further, the plurality of elongated rod members are generally independently translatable relative to each other (and relative to the instrument axis) in order to capture an accurate surface topography of the defect region.

In accordance with another embodiment of the present disclosure, an exemplary device that includes graft harvesting functionality is provided, generally including an elongated shaft and a detachable cutting member mounted with respect to the elongated shaft and operative to form a cavity or void region. In exemplary embodiments of the present disclosure, the disclosed device is adapted to form a cavity/void region of a predetermined geometry and/or depth. The exemplary device may further include a plurality of elongated rod members for capturing a topography, e.g., a peripheral surface topography, of the anatomical location in proximity to the intended or actual location of the cavity/void region, a broach member that may advantageously include structural feature(s) for at least one of cleaning and smoothing the periphery of the cavity/void region, and a hammer mechanism configured to slide relative to the axis of the shaft.

In accordance with another embodiment of the present disclosure, an exemplary instrument for removing material from a defect region is provided. The exemplary instrument generally includes a template configured and dimensioned to receive a mounting track. The exemplary instrument generally further includes a cutter configured and dimensioned to be inserted into the template. In particular, the cutter can include a travel indication feature for indicating a cutter position within the template. The template can include a peripheral template track for receiving placement of the mounting track, which further facilitates placement and anchoring of the template relative to an anatomical structure. The travel indication feature can be, e.g., a bushing, and the alignment of a template outer periphery with a travel indication feature outer periphery can indicate the cutter position within the template.

In accordance with yet another embodiment of the present disclosure, an exemplary method for defect repair is provided, generally including the steps of establishing a referential orientation of an instrument relative to an anatomical location, capturing a surface topography of the anatomical location of the defect region (e.g., a complete/entire surface topography), forming a defect region cavity that defines a cavity region geometry in the anatomical location, and using the captured surface topography of the anatomical location of the defect region to identify a donor location and/or graft source with a complementary surface topography as a harvest region or source of graft material for a plug to fill the defect region cavity. The cavity region geometry may be predefined according to the disclosed method. The graft material may be an allograft, autograft and/or synthetic material.

According to exemplary embodiments of the disclosed method, the defect region cavity is generally formed with a predefined depth and is formed at substantially a right angle relative to the axis of the instrument used to form such defect region cavity. The exemplary method generally further includes using a detachable broach member for cleaning and/or smoothing a peripheral wall associated with the defect region cavity and using a plurality of elongated rod members for capturing a surface topography of the anatomical location in proximity to the defect region cavity. Further still, the exemplary method generally includes using a cutter to obtain a graft plug from a harvest region or source of graft material (e.g., autograft, allograft and/or synthetic material), using a cutter guide to trim the graft plug to a predefined depth, using an axially movable member (e.g., a structure that also functions as the broach member) to eject the graft plug from the cutter, and introducing the graft plug into the defect region cavity. In general, the defect region cavity may be advantageously formed using a template having a predefined opening geometry. The graft plug is typically obtained using a cutter that defines a cutting geometry in which the predefined opening geometry of the template and the cutting geometry of the cutter correspond (or substantially correspond) to each other.

In accordance with embodiments of the present disclosure, an exemplary instrument for capturing a surface topography of an anatomical location is provided that generally includes a plurality of elongated rod members and a locking mechanism for releasably securing the plurality of elongated rod members relative to each other. The plurality of elongated rod members can generally be oriented to capture a surface topography of the anatomical location and can be independently translatable relative to each other. The surface topography of the anatomical location includes a combination of a peripheral surface topography and a central surface topography of the anatomical location of a defect region. In some embodiments, the plurality of elongated rod members can include a color variation thereon to indicate an effectiveness of the plurality of elongated rod members to capture the surface topography of the anatomical location.

In accordance with embodiments of the present disclosure, an exemplary instrument for removing material from a defect region is provided that generally includes a template and a detachable adapter. The template generally defines a predetermined geometry, e.g., an asymmetrical geometry, a symmetrical geometry, and the like, and can be configured to be driven into an anatomical location. The detachable adapter generally includes a plurality of elongated rod members for capturing a surface topography surrounding the defect region.

The exemplary instrument generally includes a driving mechanism and a cutter. The driving mechanism can be, e.g., a hammer mechanism, a crank-actuated mechanism, combinations thereof, and the like. The driving mechanism drives the template into the anatomical location. As the driving mechanism drives the template into the anatomical location, the detachable adapter can capture the surface topography surrounding the defect region. The crank-actuated mechanism generally includes a screw for anchoring of the template to the defect region and an actuator for driving the template into the anatomical location. The cutter can form a defect region cavity, e.g., a smooth defect region cavity, a stepped defect region cavity, and the like. The stepped defect region cavity can create a press fit between the stepped defect region cavity and a donor plug being implanted in the stepped defect region cavity.

In accordance with embodiments of the present disclosure, an exemplary assembly for defining an implant region is provided that generally includes a punch member and a guide member. The guide member can be adapted to be detachably engaged relative to the punch member. The guide member generally defines at least one aperture that can be configured and dimensioned to permit drill bit passage therethrough.

The punch member and the guide member generally cooperatively define a mechanism whereby the guide member can be detachably mounted relative to the punch member. The mechanism can be at least one of, e.g., a slide mechanism, a keying feature, and the like. Repositioning of the guide member relative to the punch member generally facilitates a cutting action at a distinct location relative to the punch member. The guide member defines a mounting mechanism to facilitate interaction with an ancillary device. The ancillary device can be, e.g., a slap hammer, a crank-actuated mechanism, and the like. The mounting mechanism can be an internally threaded aperture. The punch member can define a peripheral cutting edge selected from a group of, e.g., a clean cutting edge, a serrated cutting edge, and the like. The punch member generally includes an inner wall that defines an interior cutting region. The interior cutting region defined by the inner wall of the punch member can include a geometry selected from a group of, e.g., an oval design, a racetrack design, a pear-shaped design, and the like.

In accordance with embodiments of the present disclosure, an exemplary method for forming an implant region is provided that generally includes providing an assembly. The assembly includes a punch member and a guide member that is adapted to be detachably mounted with respect to the punch member. The guide member can define at least one cutting aperture. The method generally includes introducing a cutting member through the at least one cutting aperture to define a first cut in an anatomical location. The method further includes repositioning the guide member relative to the punch member so as to reposition the at least one cutting aperture relative to the punch member. The method includes reintroducing the cutting member through the at least one cutting aperture for defining a second cut in the anatomical location.

The exemplary method includes removing the guide member from the punch member and introducing a further cutting member to an internal region defined by the punch member. The further cutting member generally includes a bushing that controls a cutting depth thereof. Embodiments of the present disclosure are also directed to a kit including a plurality of assemblies described herein, the plurality of assemblies being of different dimensions.

In accordance with embodiments of the present disclosure, an exemplary graft harvesting device is provided that generally includes an elongated shaft and a cutting member. The cutting member can be mounted with respect to the elongated shaft and can be operative to form a harvest cavity of a predetermined geometry, e.g., an asymmetrical geometry, a symmetrical geometry, and the like. The exemplary device generally includes a plurality of elongated rod members for capturing a peripheral surface topography of the anatomical location in proximity to a defect region cavity. Further, the exemplary device generally includes a locking mechanism for releasably locking the plurality of elongated rod members in a desired relative orientation.

The plurality of elongated rod members can be actuated manually and/or electronically to translate against a surface of the anatomical location. The plurality of elongated rod members generally include a color variation thereon to indicate an effectiveness of the plurality of elongated rod members to capture the peripheral surface topography of the anatomical site. The exemplary device generally includes a driving mechanism, e.g., a hammer mechanism, a crank-actuated mechanism, combinations thereof, and the like. The crank-actuated mechanism generally includes a platform for securing a donor cartilage thereon. The platform can be translatable relative to the cutting member. The crank-actuated mechanism includes an actuator for driving the donor cartilage into the cutting member.

In accordance with embodiments of the present disclosure, an exemplary method for capturing a surface topography of an anatomical location is provided that generally includes establishing a referential orientation of an instrument relative to the anatomical location. The instrument generally includes a plurality of elongated rod members oriented to capture the surface topography of the anatomical location and including a color variation thereon to indicate an effectiveness of the plurality of elongated rod members to capture the surface topography of the anatomical location, and a locking mechanism for releasably securing the plurality of elongated rod members relative to each other. The exemplary method generally includes pressing the instrument against the anatomical location to capture the surface topography.

In accordance with embodiments of the present disclosure, an exemplary method for removing material from a defect region is provided that generally includes establishing a referential orientation of an instrument relative to the defect region. The instrument generally includes a template defining a predetermined geometry configured to be driven into an anatomical location and a detachably adapted including a plurality of elongated rod members for capturing a surface topography surrounding the defect region. The exemplary method generally includes driving the template into the defect region. In some embodiments, the method includes securing the template to the anatomical location with the defect region with, e.g., screws, K-wires, and the like. In some embodiments, the method includes partially driving the template into the defect region and securing the template to the defect region with, e.g., screws, K-wires, and the like. The exemplary method further includes capturing the surface topography surrounding the defect region simultaneously with driving the template into the defect region. Further, the exemplary method includes forming a defect region cavity, e.g., a smooth defect region cavity, a stepped defect region cavity, and the like, with a cutter.

In accordance with embodiments of the present disclosure, an exemplary method for harvesting a donor plug is provided that generally includes providing a graft harvesting device. The graft harvesting device generally includes an elongated shaft, a cutting member mounted with respect to the elongated shaft and operative to form a harvest cavity of a predetermined geometry, and a crank-actuated mechanism including a platform. The exemplary method generally includes securing a donor cartilage on the platform and driving the donor cartilage into the cutting member.

In accordance with embodiments of the present disclosure, exemplary instruments and systems for accessing and removing hyaline cartilage from desired implant sites are provided. The exemplary instruments and/or systems include a punch and a guide adapted to be detachably secured to the punch. The punch defines a cutting edge around its exposed periphery. The guide defines at least one aperture sized for receipt of a cutting member. The guide can also include means for cooperating with an ancillary device, e.g., a slap hammer, a crank-actuated mechanism, and the like, to facilitate engagement with and removal from an anatomical site. The ancillary device can typically be detachably coupled to the guide during use. The guide can be advantageously adapted to be repositioned relative to the punch such that the aperture can be relocated relative to the anatomical location. For example, the guide can be rotated by approximately 180° relative to the punch to allow introduction of the cutting member at a different controlled location relative to the anatomical location. The guide can then be removed from the punch to allow a clean-up cutting action in the region defined by the punch.

The exemplary instruments and/or systems of the present disclosure can be used with complementary instruments and/or systems to implant cartilage grafts, e.g., to fill osteochondral defects. Exemplary complementary instruments and systems are provided in a PCT application entitled “Instruments, Methods and Systems for Harvesting and Implanting Cartilage Materials,” which published as WO 2011/008968 A1.

The exemplary apparatus and/or systems may be used in connection with mapping techniques and systems of the type set forth in a PCT application entitled “Systems, Devices and Methods for Cartilage and Bone Grafting,” which published as WO 2009/154691 A9 (corrected version). Thus, in exemplary embodiments of the present disclosure, a clinician may be guided in his use of graft harvesting instrumentation by articular joint surface mapping data in locating/identifying harvest sites for “best fit” grafts, i.e., grafts that exhibit desired geometric and/or surface attributes for use in particular implantation site(s). Alternatively, the clinician may locate appropriate graft harvesting sites independent of such mapping techniques/systems. For purposes of the present disclosure, reference is made to the noted PCT application (WO 2009/154691 A9) for purposes of advantageous data mapping systems and techniques that may be employed with the disclosed instruments/systems and associated methods.

In exemplary implementations of the disclosed instruments/systems—which are adapted for use in defining a desired implant site—one or more of the following features/functionalities may be utilized in conjunction with the disclosed instruments/systems: (i) means for establishing referential orientation of instrumentation relative to an anatomical location or defect, e.g., a locking cannula assembly; (ii) means for capturing information concerning surface contour of an anatomical location, e.g., a surface contour tool featuring a plurality of circumferentially spaced, axially translatable rod/pin members and a centrally located plunger member for positioning within a defect, the surface contour tool adapted to key to a cannula assembly, or a balloon member surrounding a defect insert that is adapted to receive a curing agent; (iii) means for excising a plug from a defect plug material, such plug exhibiting a geometry that substantially conforms to the surface topography surrounding the defect site and that substantially conforms to the geometry of the defect itself, e.g., a cutting tool associated with a surface contour tool that is adapted to key to a cannula assembly; and (iv) means for implanting an excised plug in a defect.

In exemplary implementations of the disclosed instruments/systems, one or more of the following additional features/functionalities may be utilized in conjunction with the disclosed instruments/systems: (i) means for accessing a defect region-of-interest at an angle relative to an elongated shaft (e.g., approximately 90°), wherein a probe tip can be associated with a pin that moves within a control member (e.g., defect template) associated with a handle member; (ii) means for effectuating cutting functionality at an angle relative to an elongated shaft (e.g., approximately 90°), wherein the cutting blade can be adapted for movement relative to a distally-located housing between a recessed/shielded orientation and an operative orientation; and (iii) means for driving the cutting blade at an angle relative to an elongated shaft (e.g., approximately 90°), e.g., a bevel gear drive mechanism, a rotating vane mechanism, and/or a belt/pulley mechanism.

In exemplary implementations of the disclosed instruments/systems, one or more of the following further clinical features/functionalities may be utilized in conjunction with the disclosed instruments/systems: (i) means for pointing to a defect location; and (ii) means for effectuating cutting functionality at the desired defect location, wherein the foregoing functionalities are achieved utilizing in part a “four-bar” linkage mechanism.

Thus, the exemplary instruments, systems and associated methods described herein provide efficient, effective and reliable access to desired cartilage sites, removal of desired cartilage tissue and selection of donor cartilage sites or sources of graft material (allograft, autograft and/or synthetic) which geometrically match (or substantially match) an associated cavity region, and facilitate cartilage access and/or removal in a minimally invasive manner.

Other objects and features will become apparent from the following detailed description considered in conjunction with the accompanying drawings. It is to be understood, however, that the drawings are designed as an illustration only and not as a definition of the limits of the invention.

### BRIEF DESCRIPTION OF THE DRAWINGS

To assist those of skill in the art in making and using the disclosed instruments, methods and systems, reference is made to the accompanying figures, wherein:

FIG. 1 shows a perspective view of an exemplary trial member according to the present disclosure;

FIG. 2 shows a perspective view of an exemplary trial member according to the present disclosure;

FIG. 3 shows a perspective view of an exemplary trial member according to the present disclosure;

FIG. 4 shows a side view of an exemplary trial member according to the present disclosure;

FIG. 5 shows a side view of an exemplary trial member according to the present disclosure;

FIG. 6 shows a perspective view of an exemplary template and cutter according to the present disclosure;

FIG. 7 shows a perspective view of an exemplary template and defect region according to the present disclosure;

FIG. 8 shows a perspective view of an exemplary template according to the present disclosure;

FIG. 9 shows a perspective, exploded view of an exemplary template according to the present disclosure;

FIG. 10 shows a side view of an exemplary template and driving mechanism according to the present disclosure;

FIG. 11 shows a perspective view of an exemplary template assembly in cooperation with an orthopedic slap hammer for use according to the present disclosure;

FIG. 12 shows a perspective view of an exemplary template assembly brought into contact with a desired anatomical location according to the present disclosure;

FIG. 13 shows a perspective view of an exemplary template assembly with the slap hammer disengaged and a cutter tool or drill bit in position to be inserted through an aperture formed in the template assembly according to the present disclosure;

FIG. 14 shows a perspective view of an exemplary template assembly with a guide removed from a punch member according to the present disclosure;

FIG. 15 shows a perspective view of an exemplary template assembly with a guide repositioned relative to a punch member and with a cutter tool or drill bit in position to be inserted through a repositioned aperture formed in the template assembly according to the present disclosure;

FIG. 16 shows a perspective view of an exemplary template assembly with a guide removed and with a cutter in position to be introduced to a region defined by a punch member according to the present disclosure;

FIG. 17 shows a perspective view of an exemplary template assembly with a guide removed and a cutter positioned within a region defined by a punch member according to the present disclosure;

FIG. 18 shows a perspective view of an exemplary template assembly with a guide removed and a cutter positioned within a region defined by a punch member according to the present disclosure;

FIG. 19 shows a perspective view of an exemplary template assembly reengaged with a slap hammer for removal from an anatomical location according to the present disclosure;

FIGS. 20A-E show perspective, consecutive views of forming a defect region cavity in an anatomical location with an exemplary template assembly according to the present disclosure;

FIG. 21 shows a top, perspective view of an exemplary template assembly for use according to the present disclosure;

FIG. 22 shows a bottom, perspective view of an exemplary template assembly for use according to the present disclosure;

FIG. 23 shows a perspective view of an exemplary template assembly in an implant site according to the present disclosure;

FIG. 24 shows a perspective view of an exemplary template assembly with a guide removed from a punch member according to the present disclosure;

FIG. 25 shows a perspective view of an exemplary punch member with a slap hammer disengaged and a cutter tool or drill bit in position to be inserted through an aperture formed in a cutter guide and punch assembly according to the present disclosure;

FIG. 26 shows a perspective view of an exemplary punch member with a cutter guide repositioned relative to a punch member and with a cutter tool or drill bit in position to be inserted through a repositioned aperture formed in a cutter guide and punch assembly according to the present disclosure;

FIG. 27 shows a perspective view of an exemplary punch member with a cutter guide removed and with a cutter in position to be introduced to a region defined by the punch member according to the present disclosure;

FIG. 28 shows a perspective view of an exemplary cutter positioned within a region defined by a punch member according to the present disclosure;

FIG. 29 shows a perspective view of an exemplary cutter positioned within a region defined by a punch member according to the present disclosure;

FIG. 30 shows a perspective view of an exemplary punch member and an anatomical location after removal of a cutter according to the present disclosure;

FIG. 31 shows a perspective view of an exemplary punch and guide assembly reengaged with a slap hammer for removal from an anatomical location according to the present disclosure;

FIG. 32 shows a perspective view of an exemplary anatomical location after removal of a punch and guide assembly according to the present disclosure;

FIG. 33 shows a perspective view of an exemplary template assembly according to the present disclosure;

FIG. 34 shows a perspective view of an exemplary template assembly in an implant site according to the present disclosure;

FIG. 35 shows a perspective view of an exemplary template and cutting member according to the present disclosure;

FIG. 36 shows a perspective view of an exemplary template and cutting member according to the present disclosure;

FIG. 37 shows a perspective view of an exemplary template and cutter according to the present disclosure;

FIG. 38 shows a cross-sectional, side view of an exemplary template and cutter for forming a stepped defect region cavity according to the present disclosure;

FIG. 39 shows a perspective view of an exemplary stepped defect region cavity according to the present disclosure;

FIG. 40 shows a cross-sectional, side view of an exemplary stepped defect region cavity according to the present disclosure;

FIG. 41 shows a cross-sectional, perspective view of an exemplary stepped defect region cavity and donor plug according to the present disclosure;

FIG. 42 shows a cross-sectional, perspective view of an exemplary stepped defect cavity and donor plug according to the present disclosure;

FIG. 43 shows a perspective view of an exemplary template assembly according to the present disclosure;

FIG. 44 shows a perspective view of an exemplary template assembly and cutter according to the present disclosure;

FIG. 45 shows a perspective view of an exemplary template assembly and cutter according to the present disclosure;

FIG. 46 shows a perspective view of an exemplary graft harvesting device according to the present disclosure;

FIG. 47 shows a perspective view of an exemplary cutting member and broach member of an exemplary graft harvesting device prior to insertion into a defect region cavity according to the present disclosure;

FIG. 48 shows a perspective view of an exemplary graft harvesting device during insertion of an exemplary broach member into a defect region cavity according to the present disclosure;

FIG. 49 shows a perspective view of an exemplary graft harvesting device after removal of an exemplary broach member from a defect region cavity according to the present disclosure;

FIG. 50 shows a perspective view of an exemplary graft harvesting device prior to harvesting a donor graft according to the present disclosure;

FIG. 51 shows a perspective view of an exemplary graft harvesting device post-harvesting a donor graft according to the present disclosure;

FIG. 52 shows a perspective view of an exemplary cutter guide of an exemplary graft harvesting device for trimming a donor graft to a predefined depth according to the present disclosure;

FIG. 53 shows a perspective view of an exemplary graft harvesting device ejecting a donor graft according to the present disclosure;

FIG. 54 shows a perspective view of an exemplary graft harvesting device according to the present disclosure;

FIG. 55 shows an exemplary cutting member and broach member of an exemplary graft harvesting device according to the present disclosure;

FIG. 56 shows a perspective view of an exemplary graft harvesting device according to the present disclosure;

FIG. 57 shows a cross-sectional, perspective view of an exemplary graft harvesting device according to the present disclosure;

FIG. 58 shows a perspective view of an exemplary trial device of an exemplary graft harvesting device inserted into an anatomical location according to the present disclosure;

FIG. 59 shows a perspective view of an exemplary graft harvesting device with an extending harvester punch according to the present disclosure;

FIG. 60 shows a perspective view of an exemplary graft harvesting device with an extending trimmer guide and trimmer according to the present disclosure;

FIG. 61 shows a perspective view of an exemplary graft harvesting device ejecting an implant according to the present disclosure;

FIG. 62 shows a perspective view of exemplary graft harvesting devices of various sizes according to the present disclosure;

FIG. 63 shows a perspective view of exemplary graft harvesting devices of various sizes according to the present disclosure;

FIG. 64 shows a side view of an exemplary graft harvesting device according to the present disclosure;

FIG. 65 shows a side view of an exemplary graft harvesting device according to the present disclosure;

FIG. 66 shows a flowchart of an exemplary method for defect repair according to the present disclosure;

FIG. 67 shows a diagram of an exemplary surface mapping system according to the present disclosure;

FIG. 68 shows an exemplary computing system utilized with embodiments of the present disclosure;

FIG. 69 shows a flowchart of an exemplary method to identify suitable sites for bone-cartilage grafts for repairing a defect region of a patient according to the present disclosure; and

FIG. 70 shows an exemplary arrangement of a system to acquire, process and store data according to the present disclosure.

### DESCRIPTION OF EXEMPLARY EMBODIMENTS

In accordance with embodiments of the present disclosure, an instrument is provided for capturing a surface topography of a defect region. In particular, the exemplary topographical instrument generally includes a plurality of elongated rod members and a locking mechanism for releasably securing the plurality of elongated rod members relative to each other and relative to the axis of the instrument. The plurality of elongated rod members may be configured and/or oriented to capture an entire surface topography of the anatomical location of a defect region, including a combination of a peripheral surface topography and a central surface topography. Further, the plurality of elongated rod members are generally independently translatable relative to each other in order to capture an accurate surface topography of the defect region.

In accordance with another embodiment of the present disclosure, a graft harvesting device is provided, generally including an elongated shaft and a detachable cutting member mounted with respect to the elongated shaft and operative to form a harvest cavity of a predetermined geometry. The detachable cutting member may advantageously be included in a detachable subassembly that delivers cutting functionality and potentially one or more additional functionalities. The exemplary device generally further includes a plurality of elongated rod members for capturing a surface topography of the anatomical location (e.g., a peripheral surface topography) in proximity to an existing or intended defect region cavity. The exemplary device may further include an axially movable broach member that includes structural feature(s) for cleaning and/or smoothing a peripheral wall associated with the defect region cavity, and a hammer mechanism configured to slide along the elongated shaft. The disclosed broach member may be advantageously included in the detachable subassembly that includes the cutting member, and may be axially movable relative to such cutting member.

In accordance with yet another embodiment of the present disclosure, an instrument for removing material from a defect region is provided. The exemplary instrument generally includes a template configured and dimensioned to receive a mounting track. The exemplary instrument generally further includes a cutter configured and dimensioned to be inserted into the template. In particular, the cutter can include a travel indication feature for indicating a cutter position within the template. The template can include a peripheral template track for receiving placement of the mounting track, which further facilitates placement and anchoring of the template relative to an anatomical structure. The travel indication feature can be, e.g., a bushing, and the alignment of a template outer periphery with a travel indication feature outer periphery can indicate the cutter position within the template.

Exemplary instruments and systems for accessing and removing hyaline cartilage are also provided herein. The disclosed instruments, methods and/or systems may optionally be used in connection with mapping techniques and systems of the type set forth in PCT application WO 2009/154691 A9 (corrected version) and/or instruments, methods and systems for harvesting and implanting cartilage materials of the type set forth in PCT application WO 2011/008968 A1, both of which were previously incorporated by reference. The disclosed instruments, methods and systems provide efficient, effective and reliable access to desired cartilage sites, removal of desired cartilage tissue and selection of donor cartilage sites or sources of graft material (allograft, autograft and/or synthetic) which geometrically match (or substantially match) an associated cavity region, and facilitate cartilage access and/or removal in a minimally invasive manner.

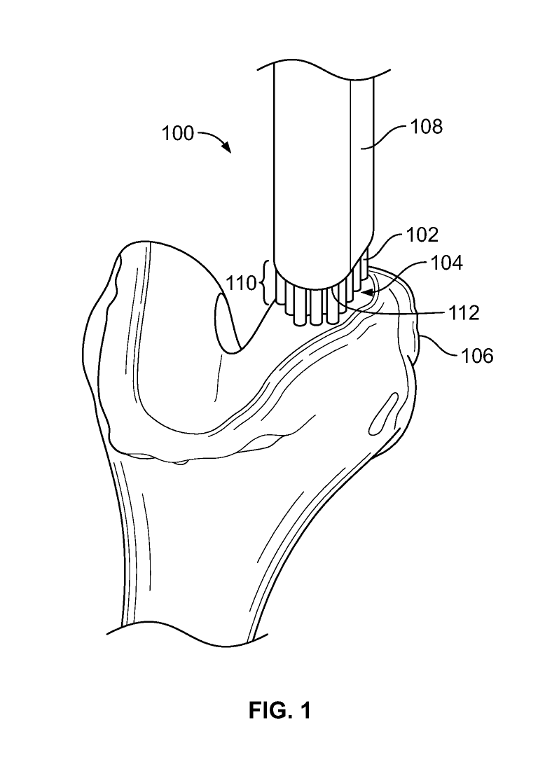

With respect to FIG. 1, an exemplary trial member 100 is presented according to an illustrative embodiment of the present disclosure. In particular, the exemplary trial member 100 generally includes a plurality of elongated rod members 102 that are movably mounted relative to each other and to the vertical axis of the trial member 100. In the illustrated embodiment, the elongated rod members 102 or pins are configured to capture the entire surface topography, e.g., a peripheral surface topography and a central surface topography, of a selected anatomical location, such as a defect region 104. However, the present disclosure is not limited by or to such structural arrangement, and extends to structural arrangements where the plurality of elongated rods members 102 or pins do not cover the full extent of the peripheral and central surface topographies. For example, the plurality of elongated rod members 102 can be configured to capture only the peripheral surface topography of a selected anatomical location, such as a defect region 104.

The plurality of elongated rod members 102 can be manufactured from a material suitable for medical purposes, e.g., stainless steel, titanium, cobalt or cobalt chrome, polymeric materials, and the like, and the selected anatomical location can be a defect region 104 of, e.g., cartilage 106 of a patient. The disclosed trial member 100 can be utilized to capture surface topography at other locations, e.g., a donor site and/or an allograft or synthetic source of potential graft material. The plurality of elongated rod members 102 can be further configured to be independently translatable relative to each other and can thereby capture the specific topography of the surface directly beneath the respective elongated rod members 102, permitting an accurate capture of the entire defect region 104 surface topography. It should be noted that the size and/or number of elongated rod members 102 depicted in FIG. 1 is for illustrative purposes only and in some exemplary embodiments, e.g., a smaller elongated rod member 102 diameter can be utilized for greater accuracy, a larger elongated rod member 102 diameter can be utilized, a greater and/or smaller number of elongated rod members 102 can be utilized depending on the area of the defect region 104 or other region of interest, combinations thereof, and the like.

Still with reference to FIG. 1, the plurality of elongated rod members 102 can generally be grouped together to provide a continuous and/or evenly distributed capture of the defect region 104 surface topography (or other region topography). Thus, in some embodiments, in addition to capturing the peripheral surface topography of the defect region 104, the exemplary trial member 100 further captures the central, e.g., middle, surface topography of the defect region 104.

A locking member 108 can further be implemented for automatically securing the plurality of elongated rod members 102 relative to each other and relative to the vertical axis of the device. The elongated rod members 102 can generally be aligned in substantially parallel, e.g., in substantially aligned paths/conduits, to ensure accuracy of the captured surface topography. The locking member 108 can be configured as, e.g., a rubber O-ring, an elastic band, a sheet of silicone with a plurality of predefined openings/apertures positioned to accommodate passage of the elongated rod members 102 therethrough, a mechanical lock, and the like. As would be apparent to those of ordinary skill in the art, the locking member 108 generally provides radial resistance, e.g., a friction fit, to releasably lock the elongated rod members 102 in place in order to accurately capture the surface topography of the defect region 104.

In particular, while the trial member 100 is being lowered in proximity to the defect region 104 in order to capture the surface topography, the elongated rod members 102 can be free to independently translate along a vertical axis running the length of each elongated rod member 102 relative to each other and the locking member 108. In some embodiments, movement of the elongated rod members 102 relative to the locking member 108 can require the user to apply a vertical force against the elongated rod members 102 to overcome the locking force, e.g., a friction fit, created by the locking member 108 against the elongated rod members 102. Once the trial member 100 has been situated in an acceptable position, the locking member 108 can be actuated and/or automatically releasably locks the elongated rod members 102 in a configuration representative of the defect region 104 surface topography. Absent a mechanical lock, the locking force applied by an O-ring, elastic band or silicone sheet may be overcome by applying an adequate force on a rod-by-rod basis.

As will be discussed in greater detail below, in some exemplary embodiments, the elongated rod members 102 of the trial member 100 can include a color variation along the length of each elongated rod member 102 for indicating when a slope of the cartilage 106 surface is too steep to be measured by the length of the elongated rod members 102 being implemented. The color variation, and thereby the slope of the cartilage 106 which can be measured by the trial member 100, can depend on the protrusion length 110 of the elongated rod members 102. In particular, the protrusion length 110 can be defined as the length of the elongated rod members 102 protruding from the bottom surface 112 of the locking member 108 in the direction of the cartilage 106 surface.

For example, if the protrusion length 110 is approximately 1 inch, it should be understood that the maximum slope variation which can be measured, i.e., the maximum difference in surface topography being measured, is approximately 1 inch. That is, if the trial member 100 is pressed against a cartilage 106 surface to measure its surface topography, the elongated rod members 102 can be axially translated through the locking member 108 up to 1 inch until the bottom surface 112 of the locking member 108 abuts the cartilage 106 surface. Any variation in surface topography of greater than 1 inch would therefore not be captured, unless longer elongated rod members 102 were implemented.

In order to alert a user, e.g., a surgeon, when the elongated rod members 102 may be insufficient for measuring the surface topography of the cartilage 106, in some embodiments, the top end of the elongated rod members 102 opposing the end defining the protrusion length 110 can include a color variation. In particular, when the protrusion length 110 has been almost fully translated into the locking member 108, e.g., approximately 0.875 inches has been translated into the locking member 108, an equal length of the elongated rod members 102 can protrude from the top surface (not shown) of the locking member 108. The surface of the top end of the elongated rod members 102 past the 0.875 inch mark can include a color variation, e.g., red paint, colored markings, and the like, indicating to the user that the full protrusion length 110 has almost been translated into the locking member 108. Thus, if the protrusion length 110 is approximately 1 inch and the color variation has been reached before the desired surface topography has been fully captured, the user can, e.g., utilize a trial member 100 with longer elongated rod members 102 (such as 1.5 inches, 2 inches, 2.5 inches, 3 inches, and the like), utilize a trial member 100 for capturing a smaller surface area of the cartilage 106 which defines less topographical variations, and the like.

Once the trial member 100 has been used to capture surface topography of a defect region 104 as described herein, the trial member 100 can then be implemented for identifying an allograft and/or autograft donor location or synthetic material as a harvest region for a plug based on a complementary surface topography, e.g., matching the defect region 104 surface topography to the surface topography of a donor location. The donor location may be, e.g., a joint of the patient, an allograft joint, or a xenograft material. Alternatively, the trial member 100 may be used to contour a synthetic material.

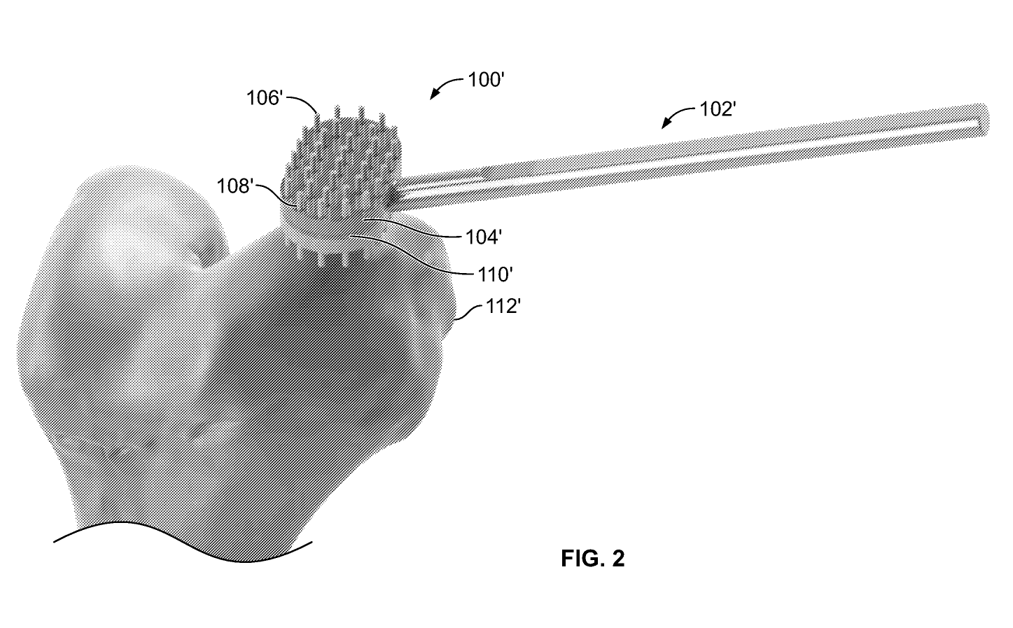

With reference to FIG. 2, an alternative exemplary embodiment of a trial member 100′ is provided. In particular, the trial member 100′ can function substantially similarly to the trial member 100 of FIG. 1. The trial member 100′ generally includes a handle 102′, a trial member body 104′ and a plurality of elongated rod members 106′. The trial member body 104′ can be fabricated from, e.g., a rubber, an elastic material, and the like, and generally includes a plurality of complementary apertures 108′ through which the elongated rod members 106′ can axially translate. In some embodiments, a friction fit of the elongated rod members 106′ within the apertures 108′ of the trial member body 104′ allows the elongated rod members 106′ to be locked in place by a locking mechanism. For example, the trial member body 104′ can include a perimeter groove 110′ configured and dimensioned to receive therein a rubber membrane, e.g., an O-ring, elastic band, and the like, which can impart a frictional force against the elongated rod members 106′. However, if a user applies a sufficient axial force to the ends of the elongated rod members 106′, the elongated rod members 106′ can translate individually relative to each other. Thus, if the trial member 100′ is pressed against the cartilage 112′ surface, the elongated rod members 106′ can axially translate within the trial member body 104′ to capture the surface topography. Although illustrated as capturing a full surface topography, i.e., a periphery surface topography and a center surface topography of the cartilage 112′, in some embodiments, the trial member 110′ can be configured to capture only the periphery surface topography of the cartilage 112′.

As described above with respect to the trial member 100 of FIG. 1, the elongated rod members 106′ can also include a visual indicator, e.g., a color variation, to alert a user when the elongated rod members 106′ may be insufficient for measuring the topography of the cartilage 112′ surface. For example, each elongated rod member 106′ can include a portion of red paint and/or colored markings indicating to the user that the elongated rod members 106′ have been translated almost the full length through the trial member body 104′. Thus, as the elongated rod members 106′ are pressed against the cartilage 112′ surface, if the length of the elongated rod members 106′ is insufficient to capture the topographical variation, the colored markings will notify the user that, e.g., longer elongated rod members 106′ should be utilized. The trial member 100′ can then be switched for, e.g., a trial member 100′ having longer elongated rod members 106′, a trial member 100′ with a smaller surface area may be used for capturing a smaller cartilage 112′ surface topography having a smaller topographical variation, and the like. For example, if the trial member 100′ includes elongated rod members 106′ of approximately 10 mm in length and the topographical variation of the cartilage 112′ surface is approximately 12 mm in length, a trial member 100′ which includes elongated rod members 106′ of approximately 15 mm in length may be utilized to accurately capture the surface topography of the cartilage 112′.

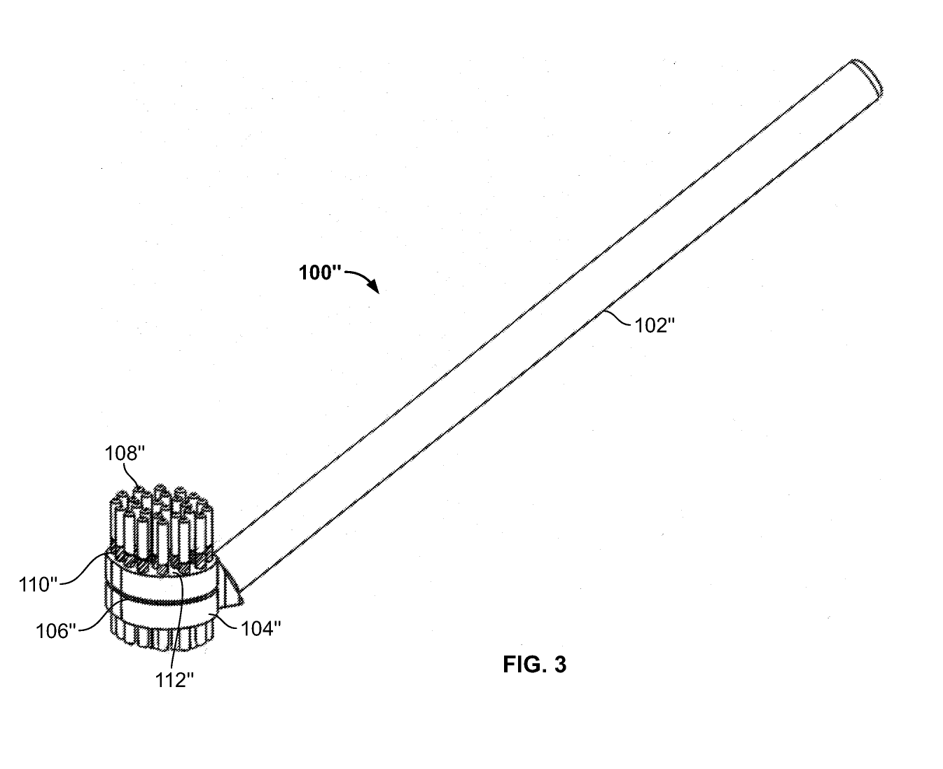

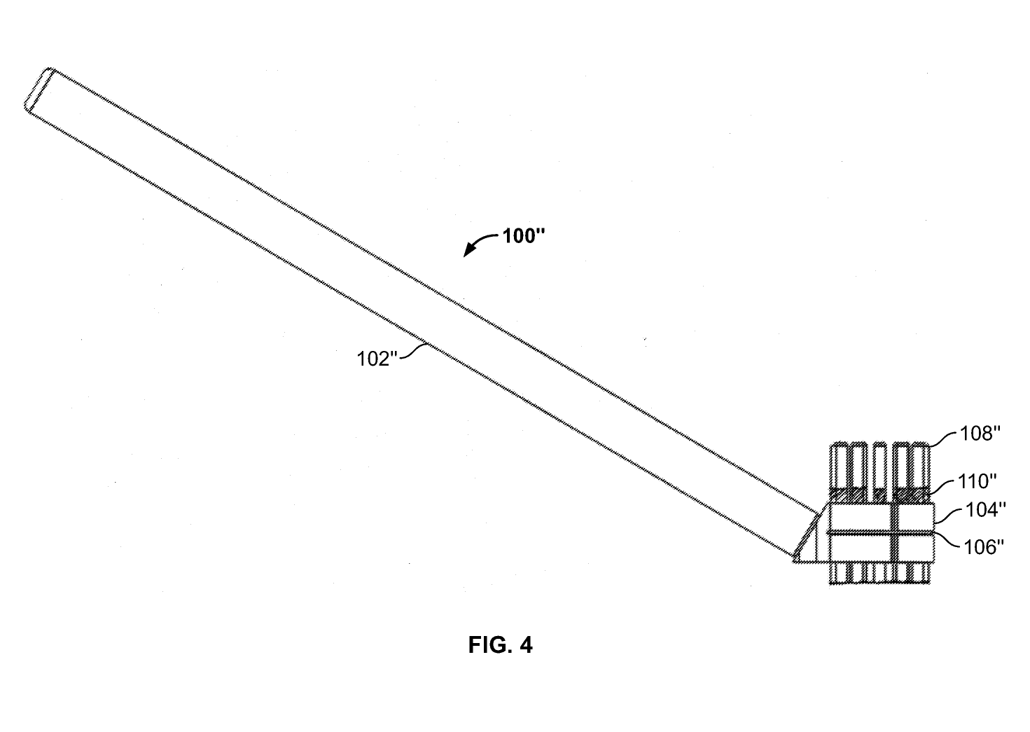

FIGS. 3 and 4 show perspective and side views, respectively, of an exemplary trial member 100″. The trial member 100″ can be substantially similar in structure and function to the trial member 100′ of FIG. 2, including a handle 102″, a trial member body 104″ including a groove 106″, and a plurality of elongated rod members 108″ or pins including a visual indicator 110″ thereon, except for the distinctions discussed herein. In particular, as illustrated in FIGS. 3 and 4, the plurality of elongated rod members 108″ of the trial member 100″ have been almost fully translated through the trial member body 104″ and the visual indicator 110″, e.g., a color variation, a texture variation, and the like, is visible. The visual indicator 110″ can generally be located within the apertures 112″ of the trial member body 104″ such that the visual indicator 110″ is not visible to the user until the elongated rod members 108″ have been almost fully translated through the trial member body 104″. In some embodiments, the visual indicator 110″ can define one color, e.g., red, or can include a plurality of colors to warn the user that the elongated rod members 108″ have been almost fully translated through the trial member body 104″. For example, the visual indicator 110″ can initially be yellow to alert the user when, e.g., 80% of the length of an elongated rod member 108″ has been translated through the trial member body 104″, and can be red to alert the user when, e.g., 90% of the length of an elongated rod member 108″ has been translated through the trial member body 104″.

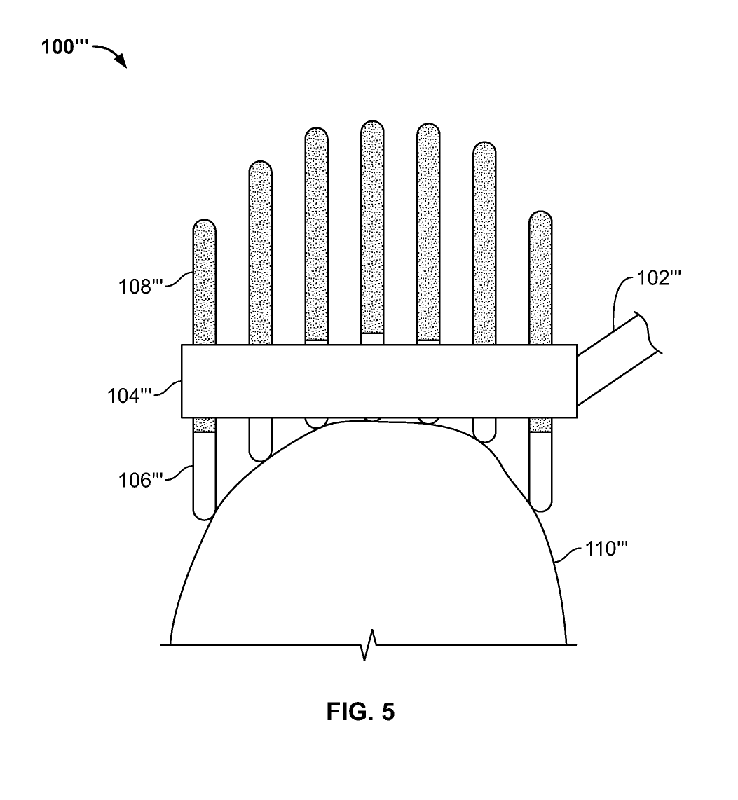

FIG. 5 shows an additional exemplary embodiment of a trial member 100′″ substantially similar in structure and function to the trial member 100″ discussed above. In particular, trial member 100′″ generally includes a handle 102′″, a trial member body 104′″, a plurality of elongated rod members 106′″, i.e., pins, and a visual indicator 108′″, such as a color variation and/or a texture variation. The elongated rod members 106′″ can be marked with the visual indicator 108′″ such that when the trial member 100′″ is brought down to and against the cartilage 110′″ surface, if any of the portions of the elongated rod members 106′″ marked with the visual indicator 108′″ are visible below the trial member body 104′″, the user will be visually alerted that the curvature of the cartilage 110′″ is too great for the trial member 100′″ to accommodate and/or measure. Thus, the visual indicator 108′″ acts as a warning to the user to utilize a trial member 100′″ which includes, e.g., elongated rod members 106′″ having a longer length, a smaller surface area for capturing a smaller surface topography, and the like. In some embodiments, rather than or in combination with the visual indicator 108′″, an auditory indicator (not shown) can be incorporated into the trial member 100′″ to provide an auditory warning signal to a user when the curvature of the cartilage 110′″ is too great for the trial member 100′″ to accommodate and/or measure.

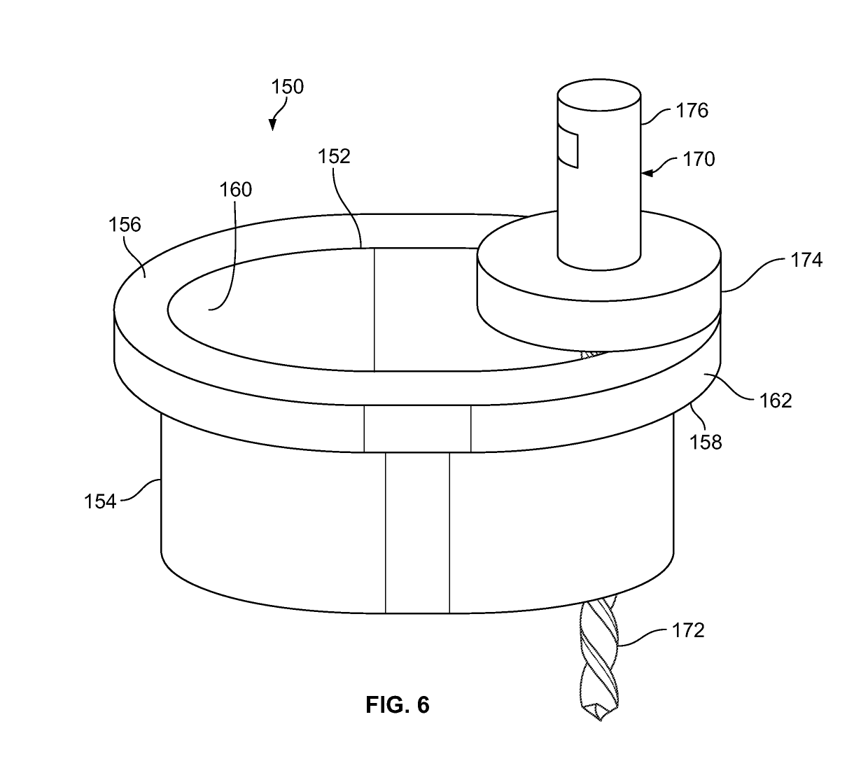

Turning now to FIG. 6, an exemplary template 150 is illustrated according to an exemplary embodiment of the present disclosure. In particular, the exemplary template 150 can be manufactured from, e.g., stainless steel, and can be utilized for forming a defect region cavity, e.g., removing material from a defect region 104 of the patient, and further generally includes a preformed geometric shape opening 152 of a desired and predefined shape. A template bottom portion 154 can be adapted for placing the template 150 onto a desired location, e.g., the cartilage 106 of a patient. In some embodiments, the template bottom portion 154 can be, e.g., a blade, a cutting member, and the like, configured for being driven into the cartilage 106 of the patient via slap hammer and/or a crank-actuated mechanism. In some embodiments, rather than or in combination with being driven into the cartilage 106, the template 150 can be secured to the cartilage 106 with, e.g., screws, K-wires, and the like. For example, the template 150 can be partially driven into the cartilage 106 to initially position the template 150 relative to the cartilage 106 and screws or K-wires can be used to further secure the template 150 to the cartilage 106. In some embodiments, rather than driving the template 150 into the cartilage 106, the template 150 can be secured to the cartilage 106 with screws or K-wires. When the template 150 is driven into the cartilage 106 of the patient, the preformed geometric shape opening 152 of the template 150 can be configured and dimensioned to define the outer periphery of the defect region cavity to be formed. A top portion 156 of the template 150 can include a peripheral protrusion 158, e.g., a lip or flange. The peripheral protrusion 158 can extend from the blade portion of the template 150 to create a stop element which controls the depth the template 150 can be driven into the cartilage 106 of the patient. As will be described below, a top surface of the peripheral protrusion 158 can also act as a stop element with respect to a cutter 170. Although illustrated as substantially oval in shape, it should be understood that the preformed geometric shape opening 152 can be configured and dimensioned as circular and/or non-circular in shape depending on the shape and size of the defect region to be removed.

FIG. 6 also illustrates a cutter 170 which generally includes a drill bit 172 and a stop element 174, e.g., a bushing, and can be utilized to form a defect region cavity by removing materials at the defect region 104. An upper shaft 176 of the cutter 170 can be adapted for engagement with a drive mechanism (not pictured), as is readily apparent to persons skilled in the art. The drill bit 172 can be, e.g., a downcutting drill bit as discussed in a U.S. patent application entitled “Orthopedic Downcutting Instrument and Associated Systems and Methods,” which published as US 2011/0238070 A1. The contents of the foregoing U.S. patent application are incorporated herein by reference.

The stop element 174 of the cutter 170 can abut the top surface of the template 150 and, in particular, the top surface of the peripheral protrusion 158, to ensure a continuous and/or even depth of the defect region cavity being formed by providing support for the cutter 170 and preventing the drill bit 172 from penetrating deeper than the desired depth. The inner side surface 160 of the preformed geometric shape opening 152 can further assist the user by guiding the drill bit 172. Of note, the stop element 174 can be sized such that the outer periphery of the stop element 174 substantially aligns with the outer periphery 162 of the top surface of the template 201 or the peripheral protrusion 158 when the drill bit 172 is in abutment (or substantial abutment) with an inner side surface 160 or wall of the preformed geometric shape opening 152. In this way, a system user can determine when the drill bit 172 has reached its “outer” travel limit based on abutment with the inner side surface 160 or wall of the preformed geometric shape opening 152 without visualization thereof. Accordingly, in such exemplary embodiments, when the side surface of the disk shaped stop element 174 and the outer periphery 162 surface of the peripheral protrusion 158 of the template 150 are aligned (or substantially aligned), it should be understood that the drill bit 172 has reached the inner side surface 160 of the preformed geometric shape opening 152. It should further be understood that the outer periphery of the top surface of the template 150, i.e., the peripheral protrusion 158, can be configured and dimensioned to match the geometry of the preformed geometric shape opening 152, thereby retaining the ability indicate the “outer” travel limit based on alignment of the outer peripheries of the stop element 174 and the top surface of the peripheral protrusion 158. Thus, the user can confidently create a defect region cavity by utilizing the visual references, e.g., the travel indication feature, of the template 150 and cutter 170 to determine where a cut is being made relative to the template 150 geometry.

As discussed above, the template 150 includes a template bottom portion 154, e.g., a blade, configured for being driven into the cartilage 106. In some embodiments, the preformed geometric shape opening 152 of the template 150 can be configured and dimensioned to receive therein a guide or adapter which, in turn, can be configured and dimensioned to receive a driving mechanism. In some exemplary embodiments, the driving mechanism for driving the template bottom portion 154 into the cartilage 106 of the patient can be, e.g., an attachable hammer mechanism, an attachable crank-actuated mechanism, combinations thereof, and the like. For example, the attachable hammer mechanism and/or crank-actuated mechanism can function substantially similarly to the hammer mechanism and/or crank-actuated mechanism described below with respect to the graft harvesting device. The attachable hammer mechanism can provide the necessary force for driving the template bottom portion 154 into the cartilage 106, while the crank-actuated mechanism can reduce the toggle effect during at least a partial insertion of the template bottom portion 154 into the cartilage 106. In some exemplary embodiments, one or more stabilizing members, e.g., K-wires, and the like, can be positioned within the preformed geometric shape opening 152 and into the defect region 104 to stabilize the template 150 during insertion into the cartilage 106, while reducing the damage to healthy cartilage 106 surrounding the defect region 104.

In some exemplary embodiments, the preformed geometric shape opening 152 of the template 150 can be configured and dimensioned to receive therein a guide or adapter which further includes a plurality of elongated rod members substantially similar in function to the elongated rod members 101 described above (not shown). In particular, the guide or adapter can include a plurality of complementary openings configured and dimensioned to receive therein the plurality of elongated rod members. It should be understood that the plurality of elongated rod members can be axially translatable within the complementary openings when a force is imparted against the elongated rod members. In addition, the guide or adapter can be fabricated from a material which imparts a frictional force against the elongated rod members to maintain the elongated rod members in a “captured” position until a force greater than the friction force is imparted to an end of the elongated rod members. Thus, as the template bottom portion 154 is driven into the cartilage 106, the plurality of elongated rod members can be forced against the defect region 104 cartilage. This force can axially translate the elongated rod members through the complementary openings to capture the surface topography within or around the perimeter of the template 150, i.e., the surface topography of the defect region 104. The guide or adapter can then be removed from the template 201 to perform the subsequent steps described below.

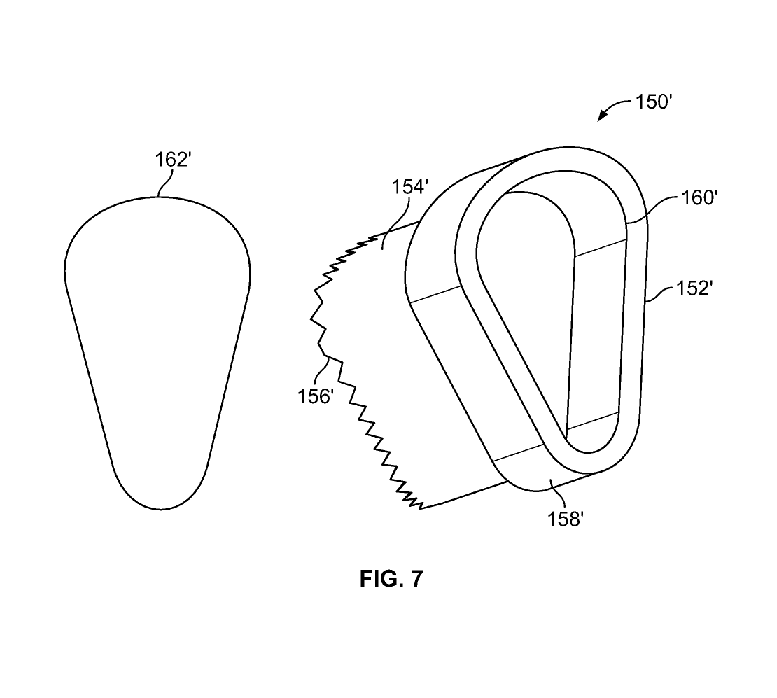

With reference to FIG. 7, an exemplary template 150′ is illustrated which generally includes a template side surface 152′ and a template bottom portion 154′, e.g., a blade defining a serrated edge 156′. The template side surface 152′ generally includes a peripheral protrusion 158′ (e.g., a lip or flange) extending therefrom to act as a stop element for controlling the depth of driving the template 150′ into the cartilage 106 and/or for controlling the depth of insertion of the cutter 170. In some embodiments, rather than or in combination with being driven into the cartilage 106, the template 150′ can be secured to the cartilage 106 with, e.g., screws, K-wires, and the like. In some exemplary embodiments, the preformed geometric shape opening 160′ of the exemplary template 150′ can be configured as non-symmetrical or asymmetrical, e.g., slightly tapered, to prevent incorrect insertion of a cartilage graft into the defect region cavity. In particular, the non-symmetrical shape of the preformed geometric shape opening 160′ creates a non-symmetrical shape of the defect region cavity. The template 150′ can be sized such that the defect region 162′ is fully captured within the asymmetrical preformed geometric shape opening 160′. It should be understood that the graft harvesting device utilized for harvesting a donor plug for implantation within the asymmetrical defect region cavity would include a cutter, i.e., a blade, defining a shape complementary to the preformed geometric shape opening 160′ to ensure proper matching between the defect region 162′ and the harvested bone-cartilage graft. Thus, when a non-symmetrical bone-cartilage graft is harvested for insertion into the non-symmetrical defect region cavity, the orientation of the bone-cartilage graft relative to the defect region cavity can be properly maintained by allowing insertion of the bone-cartilage graft only when the non-symmetrical configurations have been aligned. The non-symmetrical configuration of the template 150′ and the blade of the graft harvesting device further guarantee the correct orientation of the instruments used during the donor plug surgery.

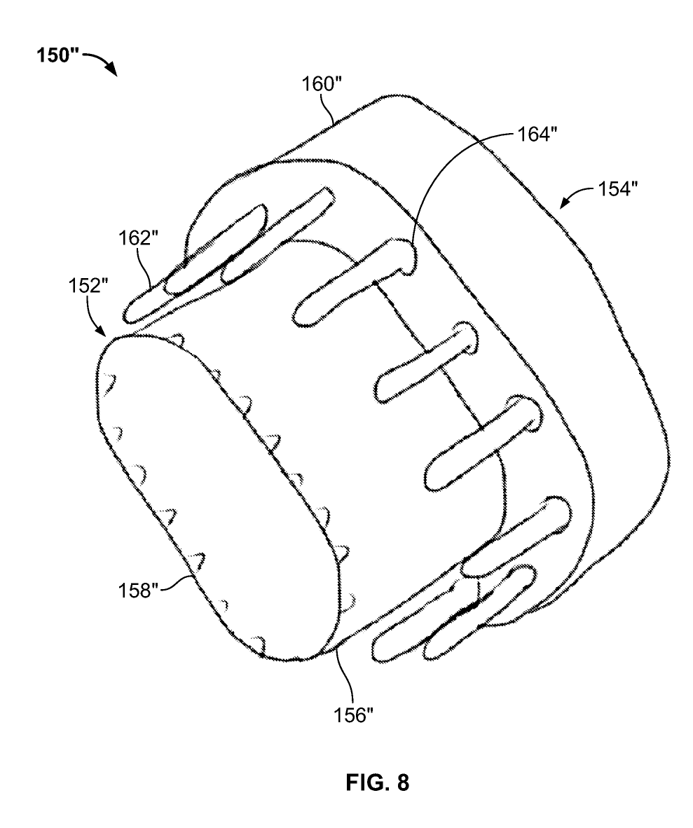

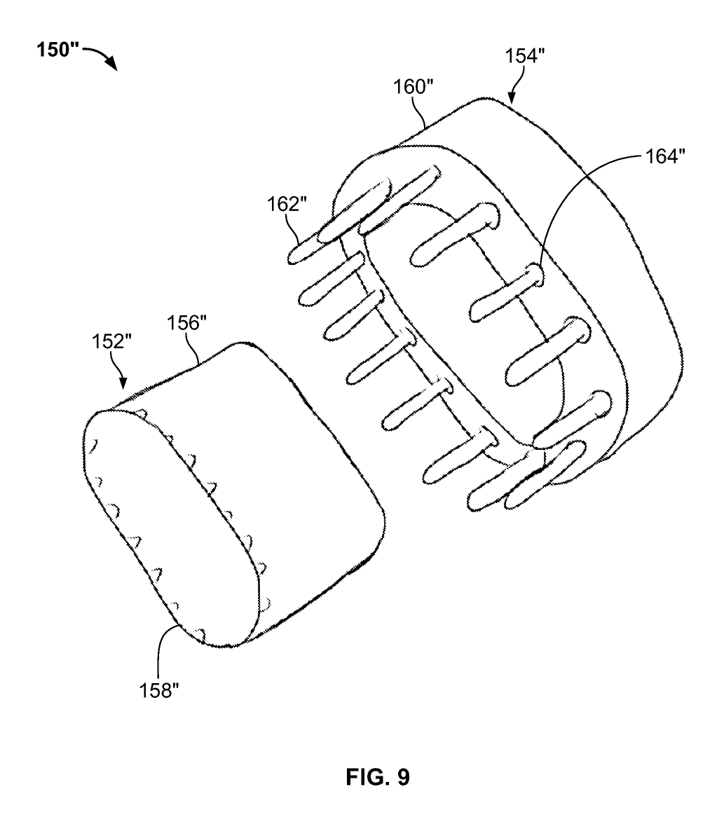

With reference to FIGS. 8 and 9, an exemplary template assembly 150″ is shown which includes a template 152″ and an adapter 154″ which can be configured and dimensioned to detachably interlock with the template 152″. The template 152″ includes a template bottom portion 156″ which includes a blade 158″, e.g., a serrated blade. The shape of the blade 158″ can be configured and dimensioned as needed to create the desired defect region cavity. The adapter 154″ includes a locking member 160″ and a plurality of elongated rod members 162″ or pins. In particular, the locking member 160″ can be configured and dimensioned to interlock with a top surface and/or edge of the template 152″ such that the template 152″ and the adapter 154″ form a substantially unified structure. The locking member 160″ can also be configured and dimensioned to overlap and create a flange extending beyond the perimeter of the template 152″ such that the plurality of elongated rod members 162″ extend down and around the template 152″. It should be understood that the elongated rod members 162″ can translate through complementary apertures 164″ formed in the locking member 160″ when an axial force is applied to the elongated rod members 162″. In addition, the locking member 160″ can be fabricated from a material which imparts a frictional force onto the elongated rod members 162″, e.g., a rubber material, an elastic material, and the like, to “capture” the position of the elongated rod members 162″ when a force is not being imparted on them.

In some embodiments, the locking member 160″ can include a locking mechanism (not shown) for locking the elongated rod members 162″ relative to the locking member 160″. The template assembly 150″ can be configured to receive a driving mechanism (not shown) for driving the template bottom portion 156″ into the cartilage 106. In some embodiments, rather than or in combination with being driven into the cartilage 106, the template 150″ can be secured to the cartilage 106 with, e.g., screws, K-wires, and the like. Thus, as the template assembly 150″ is driven into the cartilage 106 at a defect region 104, the plurality of elongated rod members 162″ can be forced against the cartilage 106 of the defect region. This force can axially translate the elongated rod members 162″ through the complementary apertures 164″ or openings to capture the surface topography at the perimeter of the defect region 104, i.e., the surface topography surrounding the defect region 104. The adapter 154″ can then be detached or removed from the template 152″ and used to substantially match the defect region 104 perimeter topography to a donor site. As described above, the template 152″ can remain in the cartilage 106 to allow the surgeon to create a defect region cavity.

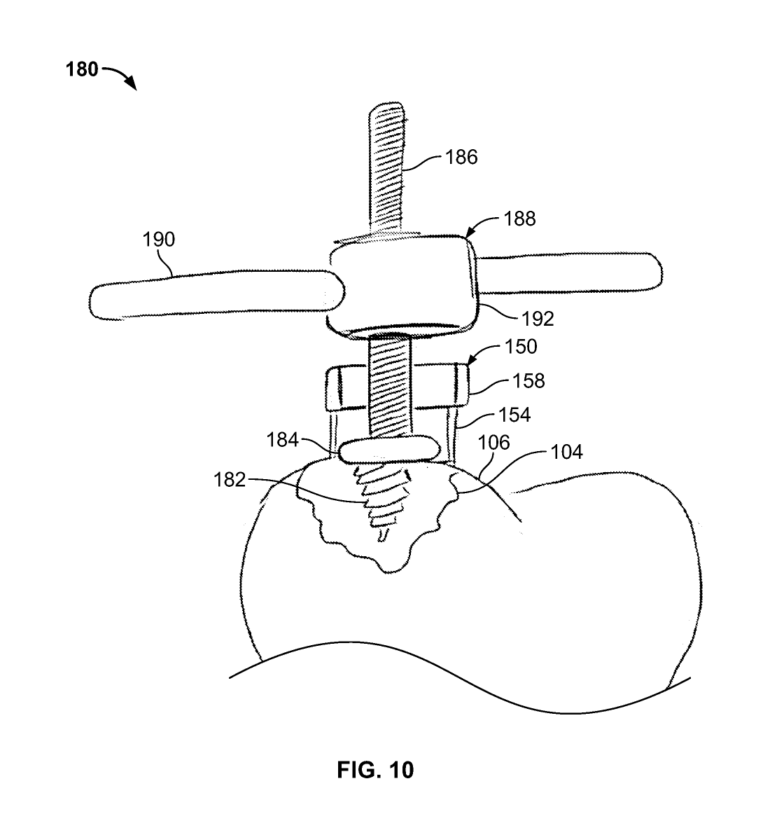

FIG. 10 illustrates an exemplary crank-actuated mechanism 180 mounted with respect to the exemplary template 150. The exemplary mechanism 180 generally includes a screw 182, a threaded rod 186 and an actuator 188. In some embodiments, the mechanism 180 also includes a screw stop element 184 positioned adjacent and fixated relative to the screw 182. In some embodiments, the threaded rod 186 can be attached to the screw 186 and/or the screw stop element 184. The template 150 can initially be positioned over the defect region 104 and the screw 182 can be driven and/or screwed into the defect region 104 to stabilize and/or position the mechanism 180 relative to the cartilage 106. It should be noted that the screw 182 can be driven into the defect region 104 only, thus preventing damage to healthy cartilage 106 surrounding the defect region 104. Optionally, a screw stop element 184 can be implemented for regulating the depth of entry of the screw 182 into the defect region 104 by abutting the top surface of the cartilage 106 when the screw 182 has reached the desired depth. Once the screw 182 has been fully driven into the defect region 104, the actuator 188 can be actuated to drive the template bottom portion 154 into the cartilage 106. In particular, the actuator handles 190 can be utilized to rotate and move the actuator body 192 down the threaded rod 186 and against the top surface of the template 150. The actuator body 192 thereby imparts a driving force against the template 150 and drives the template bottom portion 154 into the cartilage 106. After the template bottom portion 154 has been driven into the cartilage 106 to the desired depth, the mechanism 180 can be removed to perform the steps for forming the defect region cavity.

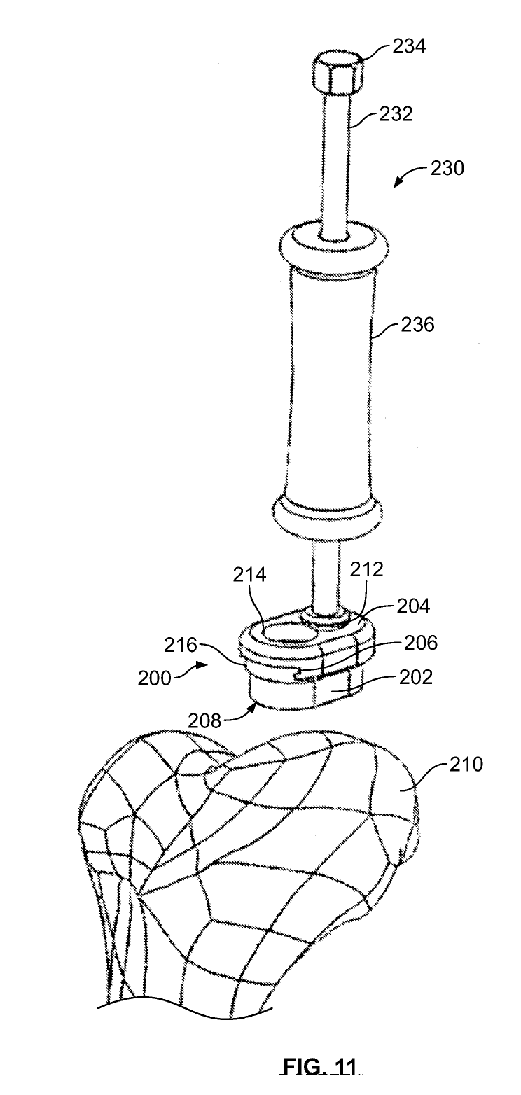

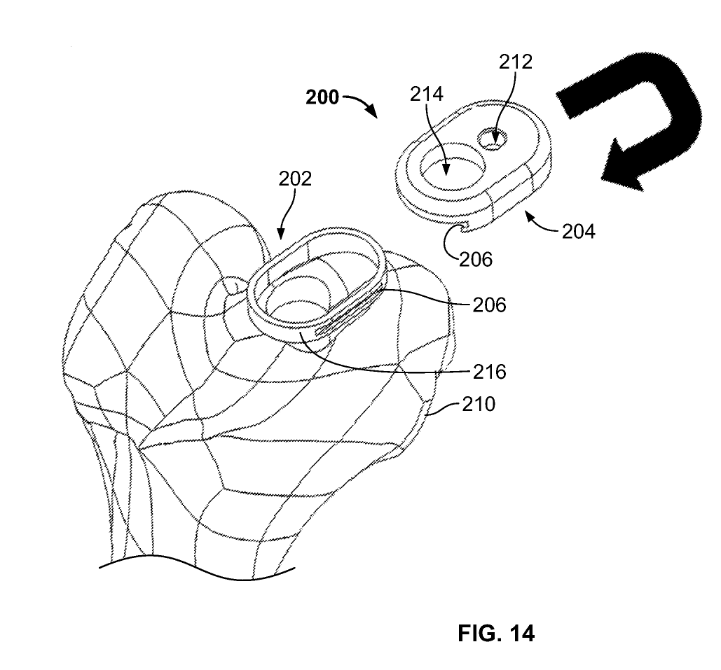

Turning now to FIG. 11, an exemplary template assembly 200, e.g., a punch and template assembly, for use in establishing a desired implant site (i.e., a defect region cavity) in is provided. Assembly 200 generally includes a punch 202 and a removable/detachable guide 204. In the exemplary embodiment depicted in the accompanying figures, the punch 202 and the guide 204 define a cooperating slide mechanism 206, whereby the guide 204 can be slid into (and out of) interlocking engagement with the punch 202 (see, e.g., FIG. 14). In some embodiments, alternative coupling mechanisms, e.g., a snap fit mechanism that permits “top loading” of the guide 204 relative to the punch 202, can be utilized. The punch 202 defines a cutting edge 208 around the exposed periphery of the punch 202 configured to be driven into the cartilage 210. The cutting edge 208 can define a “clean” cutting blade or a serrated blade (not shown), or combinations/variations thereof. Similar to the templates discussed above, the cutting edge 208 of the punch 202 can be configured and dimensioned to capture the desired defect area when driven into the cartilage 210. Thus, although illustrated as substantially oval in shape, in some embodiments, the configuration of the cutting edge 208 can be circular or non-circular. When driven into the bone or cartilage 210, the configuration of the cutting edge 208 can create a substantially complementary sized outline in the cartilage 210.

As shown in FIG. 11, assembly 200 can be adapted to cooperate with an ancillary device 230, e.g., a slap hammer, a crank-actuated mechanism, and the like, that facilitates driving the punch 202 into a desired anatomical location with minimal heat generation. Conventional slap hammers may be used, as are known to persons skilled in the art. The ancillary device 230 of FIG. 11 can be a slap hammer which generally includes an axial shaft 232, a top cap 234 and a hammer mechanism 236. The hammer mechanism 236 can be used as a handle and can further axially slide along the axial shaft 232. A user can thereby slide the hammer mechanism 236 along the axial shaft 232 to generate a force against the assembly 200 to drive the punch 202 into the cartilage 210. Detachable coupling of the device 230 relative to guide 204 can be accomplished in various ways, e.g., threading of a distal portion of the device 230 into a threaded aperture 212 formed in the guide 204. The coupled device 230 and the guide 204 can be used to drive the cutting edge 208 of the punch 202 into a desired anatomical location around a defect. In some embodiments, rather than or in combination with being driven into the anatomical location, the punch 202 can be secured or fixated to the anatomical location with, e.g., screws, K-wires, and the like.

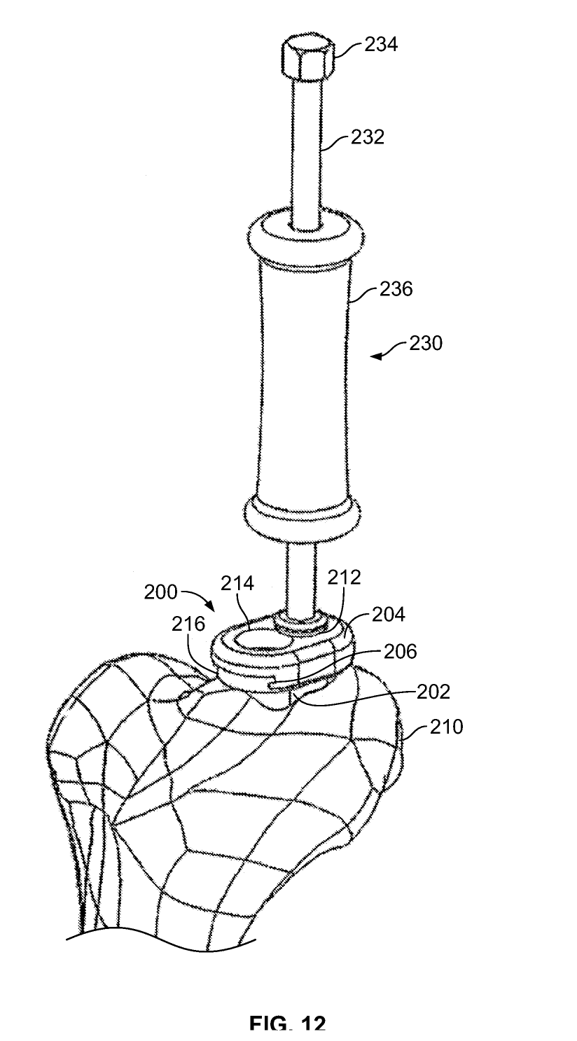

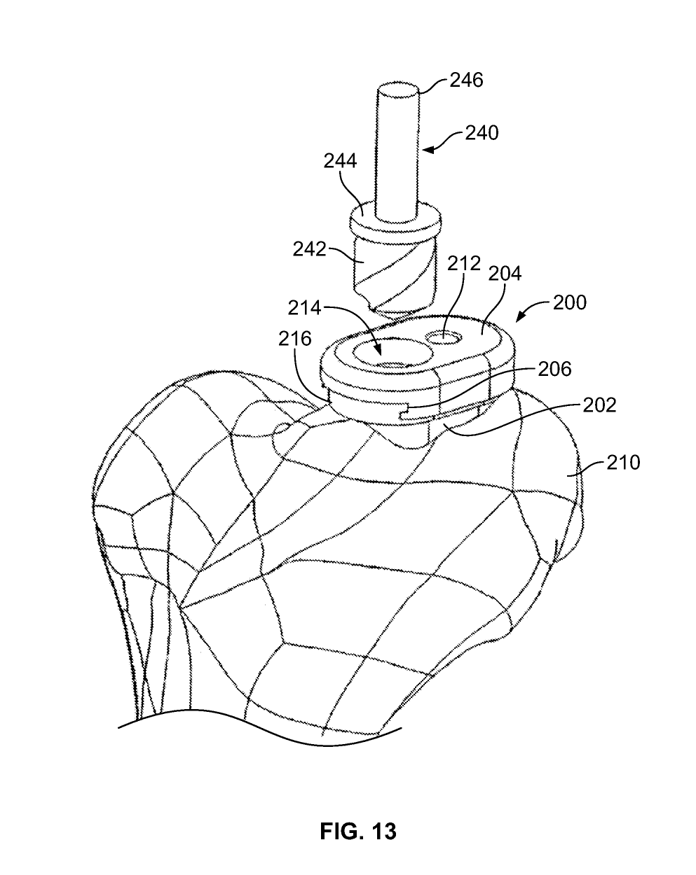

After driving the punch 202 or cutter to a desired depth in the cartilage 210, as shown in FIG. 12, the ancillary device 230, e.g., slap hammer, can be detached from the guide 204, as illustrated in FIG. 13. As will be described in greater detail below, a cutting aperture 214 formed in the guide 204 and positioned adjacent to the threaded aperture 212 can then be used to remove the damaged cartilage from the area within the perimeter of the punch 202 to form the defect region cavity. The guide 204 can thereby act as an installation and removal tool and drill guide. In some embodiments, the threaded aperture 212 can be dimensioned greater in diameter than the cutting aperture 214. The punch 202 can include a peripheral protrusion 216 which acts as a stop element to control the depth the punch 202 can be driven into the cartilage 210. In some embodiments, the peripheral protrusion 216 can aid in interlocking the punch 202 relative to the guide 204 by detachably or slidably interlocking with features of the guide 204.

The peripheral shape of the cutting edge 208 of the punch 202 can take various forms. In the exemplary embodiment of the figures provided herein, the cutting edge 208 defines a “racetrack” design. Exemplary alternative shapes include oval peripheries, pear-shaped peripheries, and the like. In addition, it is contemplated according to the present disclosure that the assembly 200 can be provided in different overall sizes for use in different anatomical circumstances. For example, a kit may be provided with multiple assemblies of varying sizes, thereby allowing the surgeon to select an appropriately sized assembly 200 for clinical use based on anatomical considerations. Thus, in exemplary implementations of the present disclosure, the surgeon would visualize the defect at the anatomical location, optionally select an appropriately sized (and potentially an appropriately shaped assembly 200) from among the available assemblies, and tap/drive the assembly 200 into the desired anatomical location relative to the defect, e.g., using an ancillary slap hammer that is coupled to the assembly.

Turning to FIG. 13, once the punch 202 has been driven to a desired depth in the anatomical location and the ancillary device 230 (e.g., slap hammer) has been decoupled and removed from the guide 204, the surgeon can introduce a cutting member 240 through a cutting aperture 214 defined in the guide 204. The cutting member 240 generally includes a cutting bit 242 that can be sized to closely cooperate with the cutting aperture 214. In addition, the cutting member 240 can include a stop 244 that, through engagement with the top face of guide 214, controls the depth to which the cutting bit will cut into the cartilage 210. The cutting bit 242 generally defines a conical distal face that serves to maintain the cutting member 240 in a desired orientation relative to guide 214 once cutting engagement with the anatomical location commences. The cutting member 240 further includes a shaft 246 for mechanical cooperation relative to a drive mechanism (not shown) for driving the cutting bit 242 into the cartilage 210. The cutting bit 242 can be dimensioned to initially remove bulk material from the defect region and, as will be discussed in greater detail below, a smaller cutting bit can be subsequently utilized to create and clean the remaining portion of the defect region cavity.

With reference to FIG. 14, after a desired “first cut” can been completed, the cutting member 240 can separated from the guide 204 with withdrawal of the cutting bit 242 from the cutting aperture 214 and the guide 104 can be removed from engagement with the punch 202 via the slide mechanism 206. As noted above, the slide mechanism 206 associated with the disclosed embodiment is merely exemplary and, in some embodiments, alternative interlocking mechanisms can be used. The guide 204 can be rotated approximately 180° relative to the punch 202 and then recoupled thereto. In this way, the cutting aperture 214 can be repositioned relative to the anatomical location to remove bulk material on the opposing side of the defect region.

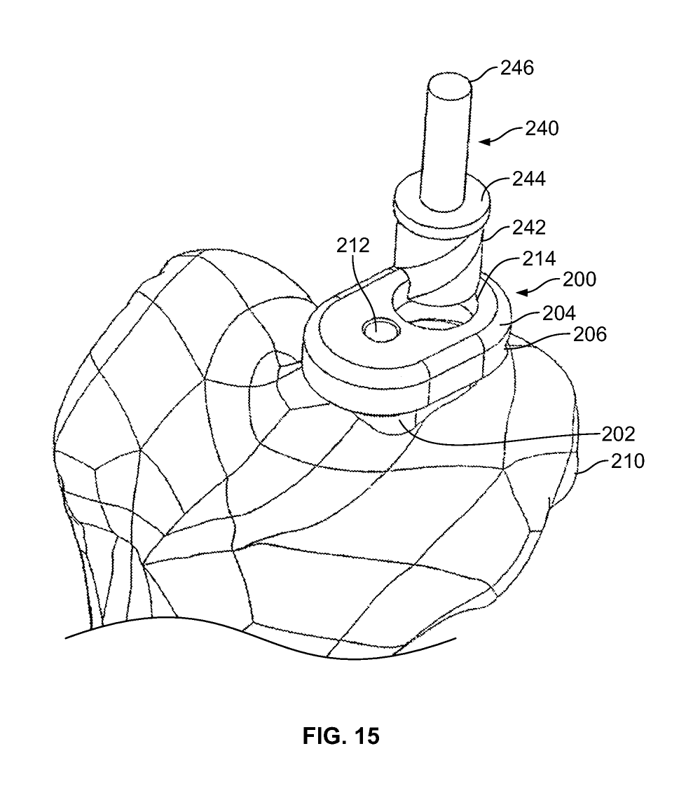

As shown in FIG. 15, the cutting member 240 can be reintroduced into the cutting aperture 214 and a second cut can be made to a desired depth into the cartilage 210 to remove bulk material. In this way, additional bulk material can removed from the anatomical location before a clean-up cutter can be used to remove the remaining cartilage 210 in the defect region within the perimeter of the punch 202.

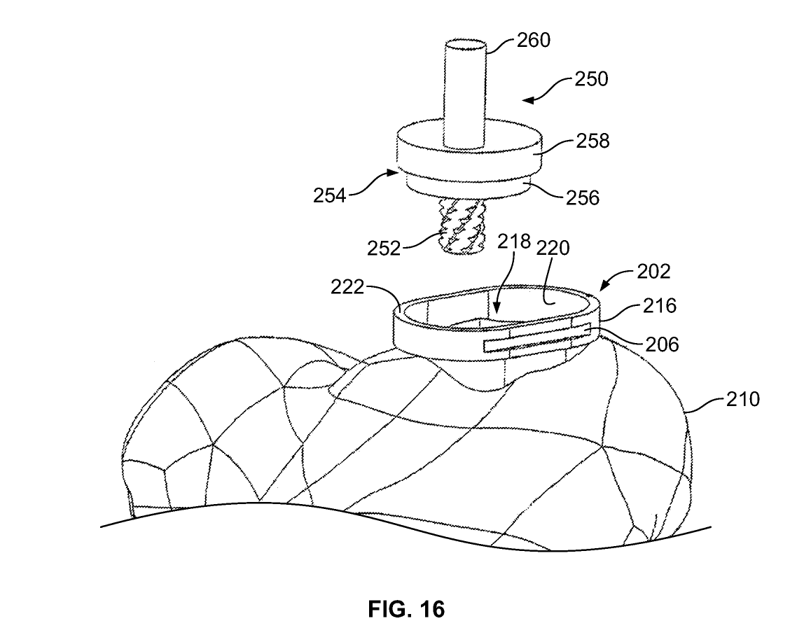

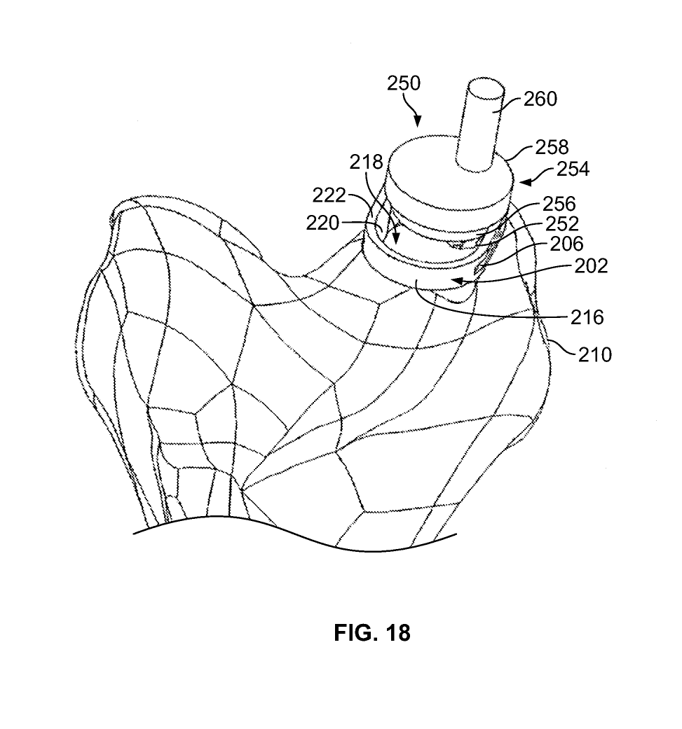

With reference to FIGS. 16-18, to clean-up the cuts made by the cutting member 230, i.e., the “coarse” cuts associated with the disclosed system/method, a clean-up cutting step can generally be undertaken. Thus, in an exemplary embodiment, the guide 204 can be removed from the punch 202 to expose a cutting region 218 within the perimeter of the punch 202. A clean-up cutter 250 can then be introduced to the cutting region 218 defined by the side wall 220 of the punch 202. The clean-up cutter 250 generally includes a cutting bit 252 and an offset bushing 254 that guides travel of the clean-up cutter 250 relative to the punch 202. The cutting bit 252 can be dimensioned smaller in diameter than the cutting member 230 and can include finer cutting edges to ensure that the remaining cartilage 210 in the defect region can be removed, while creating a smooth and uniform cut on the bottom and/or side surfaces of the defect region.

The bushing 254 generally includes a lower portion 256 that engages the inner surface of side wall 220 as the cutter 250 travels within cutting region 218. The bushing 254 also includes an offset upper portion 258 which rides along and against the top portion 222 of the side wall 220 and controls the depth to which cutting bit 252 engages the anatomical location. In some embodiments, similar to the templates discussed above, the upper portion 258 of the cutter 250 and the top portion 222 of the side wall 220 can be dimensioned such that alignment of the upper portion 258 relative to the top portion 222 can visually indicate to a user when the cutting bit 252 has reaches the inner side wall 220 of the punch 202. In general, the bushing 254 maintains the cutting bit 252 within or at the edge of the cutting region 218 without contacting the punch 202. The bushing 254 also maintains the cutting bit 252 in a substantially perpendicular position relative to the punch 202. The cutting bit 252 can be offset from the center of the bushing 254 and can visually indicate the position of the cutting bit 252 in the cutting region 218. In some embodiments, the cutting bit 252 can be centered relative to the bushing 254 diameter. The clean-up cutter 250 includes a shaft 260 extending axially from the bushing 254 and can be configured and dimensioned to mechanically interlock relative to a drive mechanism (not shown) for driving the cutting bit 252 into the cartilage 210.

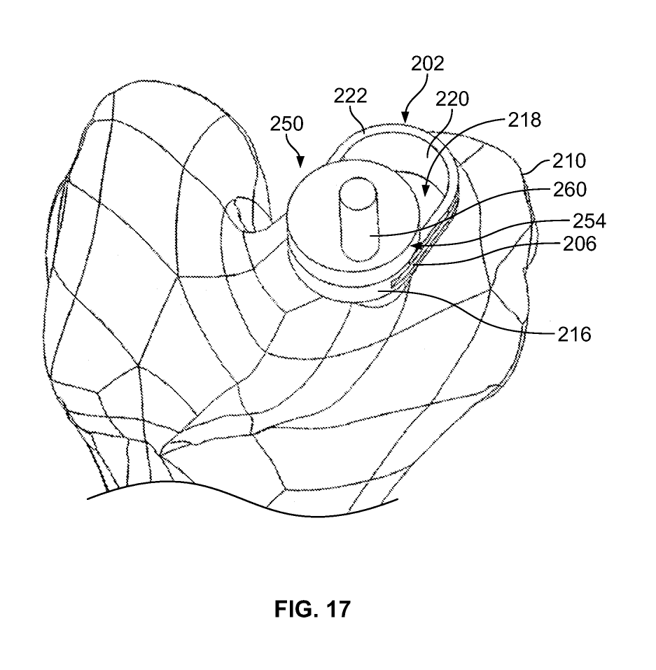

The clean-up cutter 250 advantageously functions to remove material left in the cutting region 218 by the first and second cutting actions of the cutting member 240, thereby generating a clean and uniform cut to a desired depth with a peripheral geometry defined by the side wall 220 of the punch 202. In particular, the clean-up cutter 250 creates substantially clean, planar and uniform side and bottom surfaces of the cutting region 218. In most instances, as illustrated in FIG. 17, a single instance of travel of the clean-up cutter 250 within the cutting region 218 may be effective to achieve the desired result of a clean and uniform defect region cavity.

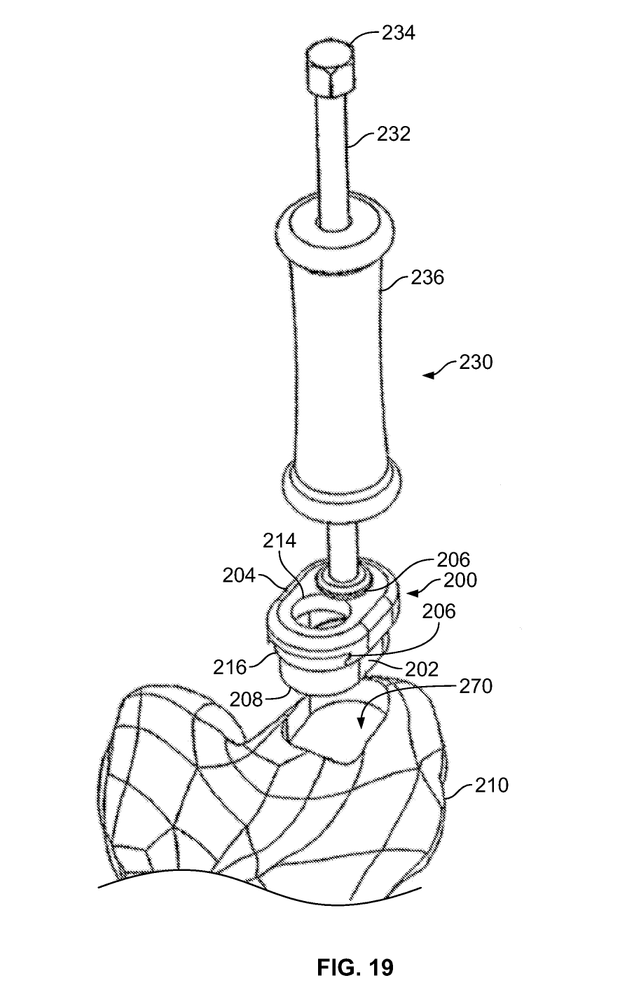

With reference to FIG. 19, after completion of the clean-up cut, the guide 204 can be reattached to the punch 202 and the ancillary device 230, e.g., a slap hammer, can be reengaged with the associated aperture 212 to facilitate removal (without twisting) of the assembly 200 from the anatomical site. For example, the hammer mechanism 236 of the slap hammer illustrated in FIG. 19 can be used to create a force away from the cartilage 210 by hammering against the top cap 234. The upward force of the hammer mechanism 236 can thereby drive the assembly 200 out of the cartilage 210. As is apparent from FIG. 19, the defect region cavity 270, i.e., the implant region, formed at the anatomical site can be clean and uniform in both peripheral geometry and depth.

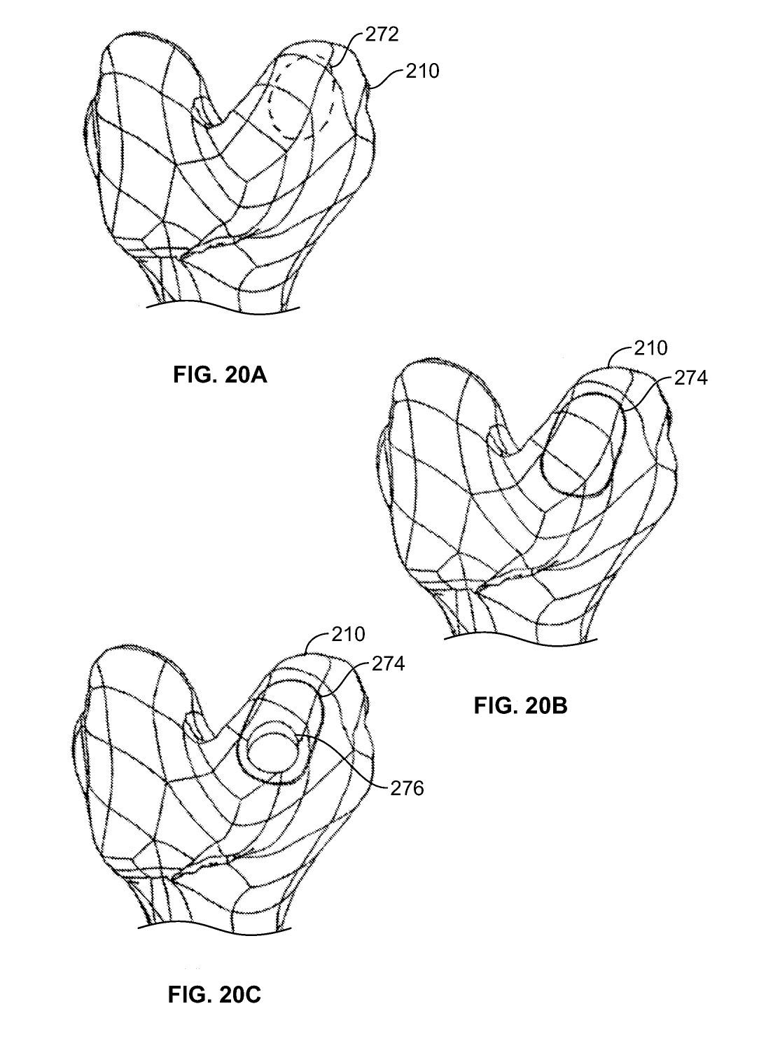

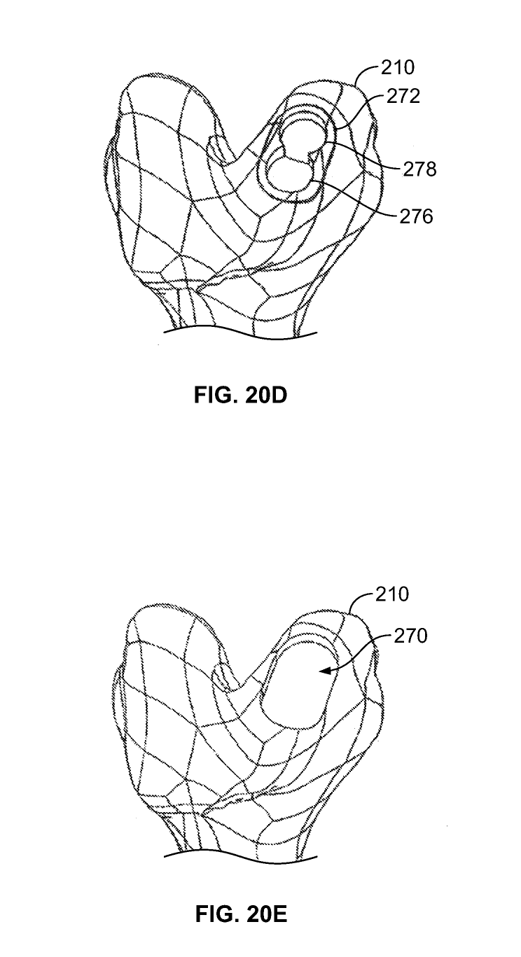

FIGS. 20A-E provides a series of five (5) schematic views that demonstrate the sequence by which the defect region cavity 270 can be formed according to exemplary implementations of the present disclosure. FIG. 20A shows the cartilage 210 prior to removal of any cartilage 210 from the defect region 272. FIG. 20B shows a peripheral cut 274 formed in the cartilage 210 after the punch 202 has been driven into the cartilage 210. FIG. 20C shows the peripheral cut 274 and a first bulk material cut 276 or drill hole formed by the cutting member 240. FIG. 20D shows the peripheral cut 274, the first bulk material cut 276 and a second bulk material cut 278 or drill hole formed by the cutting member 240 after the guide 204 has been rotated relative to the punch 202. FIG. 20E shows the final defect region cavity 270 formed after the clean-up cutter 250 has been utilized to create a uniform and clean cut to remove the remaining cartilage 210 in the defect region 272. It should be understood that the final defect region cavity 270 formed by the clean-up cutter 250 creates a substantially flat bottom surface of the defect region cavity 270 configured and dimensioned to receive a complementary implant graft. As noted above, in addition to the systems and/or methods discussed herein, the defect region cavity 270 or implant region may receive an implant using other advantageous systems/methods as described, for example, in the PCT applications WO 2009/154691 A9 (corrected version) and WO 2011/008968 A1, which have been previously incorporated herein by reference.

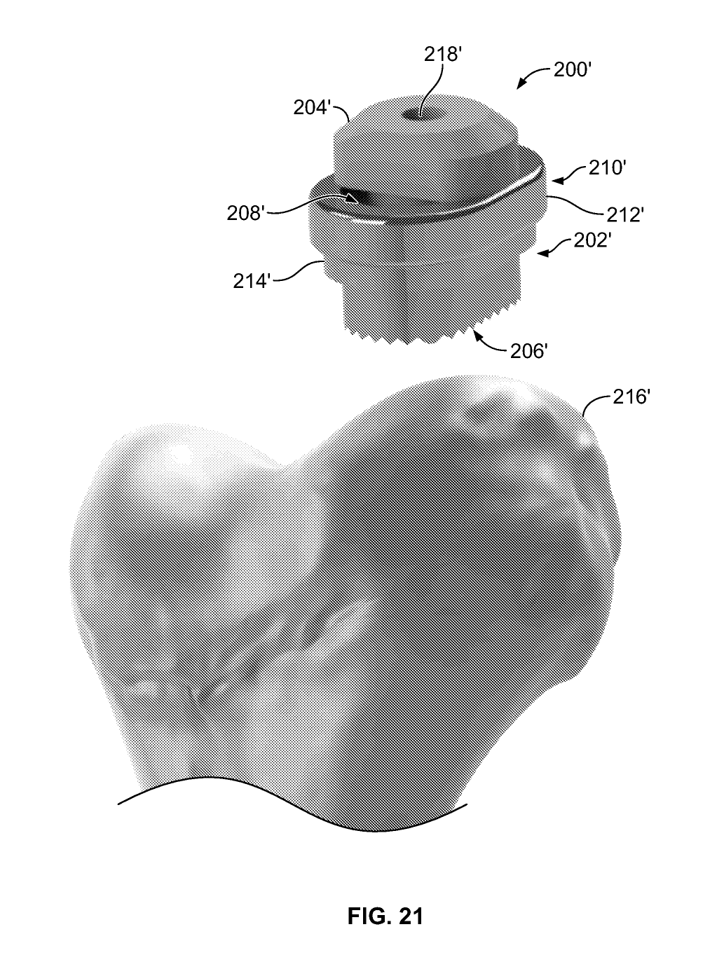

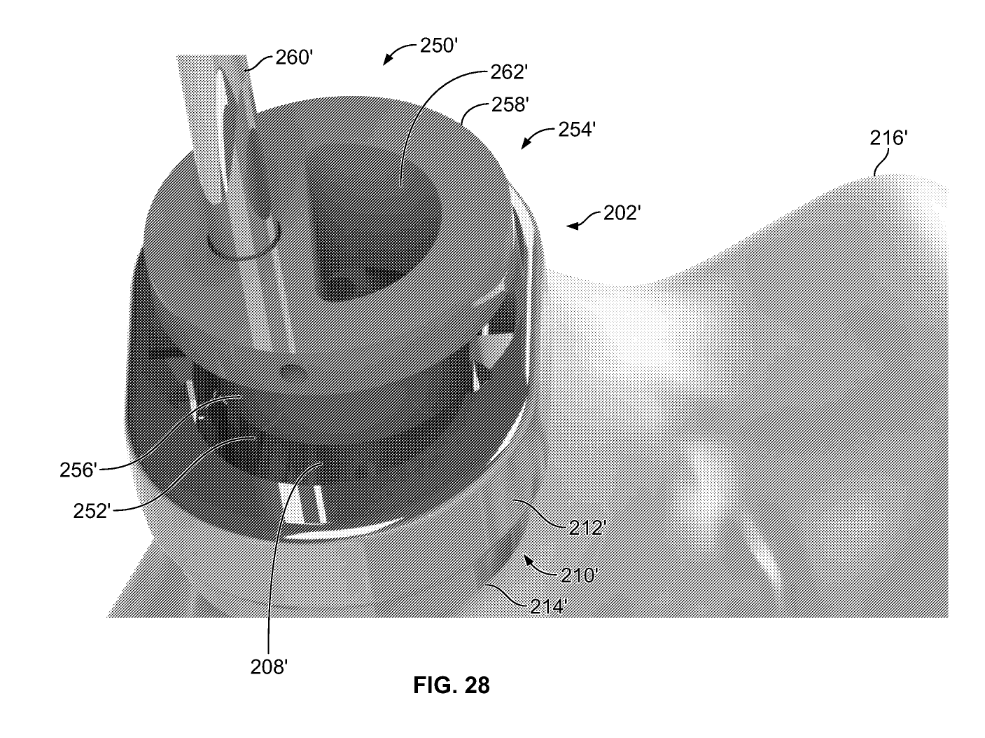

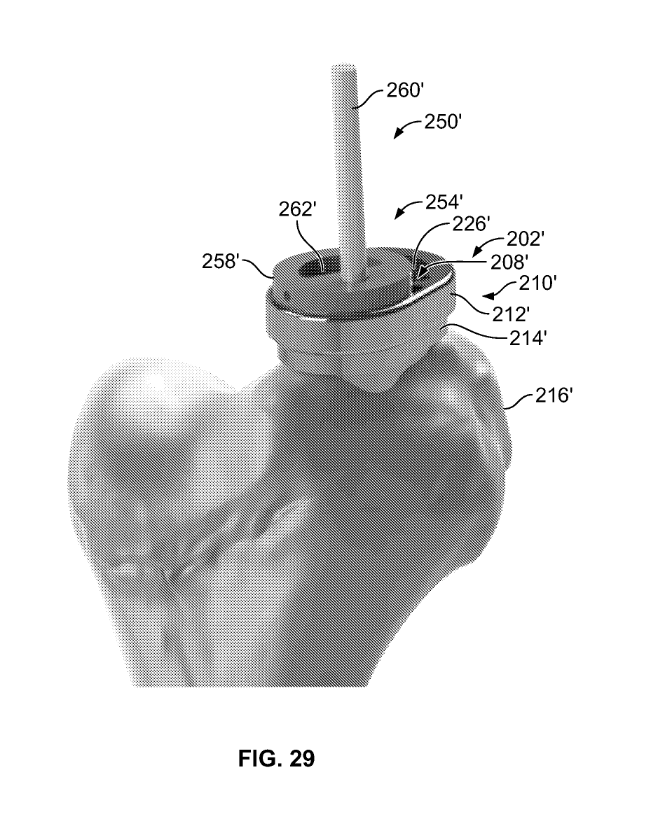

In accordance with further embodiments of the present disclosure, an exemplary punch/template assembly, i.e., a template assembly 200′, for use in establishing a desired implant site and creating a defect region cavity is provided in FIGS. 21-32. As shown in FIGS. 1-5, in some exemplary embodiments, a trial member can be implemented to locate the desired implant site. The trial member generally includes a plurality of pins for capturing the surface topography of the surgical site. As discussed above, the plurality of pins can generally translate through complementary apertures in the trial body and can further be locked in place by a locking mechanism, e.g., a rubber membrane imparting a frictional force against the pins, and the like.

With reference to FIG. 21, an exemplary assembly 200′ is provided, generally including a punch 202′ and a guide 204′. The punch 202′ defines a cutting edge 206′ around an exposed periphery of the punch 202′. The cutting edge 206′ can define a “clean” cutting blade (not shown), a serrated blade, or combinations/variations thereof. As discussed above, the exemplary cutting edge 206′ can define, e.g., a racetrack, oval, pear-shaped, circular, and the like, periphery. The punch 202′ and the guide 204′ can define a cooperating interlocking mechanism, e.g., a keying feature, which guides interaction between the punch 202′ and the guide 204′. For example, the guide 204′ can be inserted into the aperture 208′ (i.e., cutting area) of the punch 202′ and, in particular, the locking features 220′, e.g., protrusions, of the guide 204′ can be inserted through complementary slots 222′ in a top surface of the punch 202′ (see, e.g., FIG. 24). In some embodiments, the guide 204′ can include one or more locking features 220′ axially protruding from a bottom section of the guide 204′ which can be configured and dimensioned to be inserted into the aperture 208′ of the punch 202′. The guide 204′ can further be interlocked with the punch 202′ by rotating the guide 204′ and thereby rotating the locking features 220′ of the guide 204′ within a path 224′ on the inside of the punch 202′ (see, e.g., FIG. 24). The detachable guide 204′ therefore creates a partial extent of coverage relative to the punch 202′, i.e., the detachable guide 204′ essentially operates within a portion of the punch 202′ rather than engaging the periphery of the punch 202′.

The punch 202′ generally includes at least one peripheral protrusion 210′ extending from the punch 202′ at an upper end opposing the cutting edge 206′. As illustrated in FIG. 21, the punch 202′ can include a first peripheral protrusion 212′ or flange dimensioned greater than a second peripheral protrusion 214′ or flange to create a stepped peripheral protrusion 210′. The first peripheral protrusion 212′ can interact with the guide 204′ and/or a cutting member, while the second peripheral protrusion 214′ can act as a stop element to control the depth the punch 202′ can be driven into the cartilage 216′.

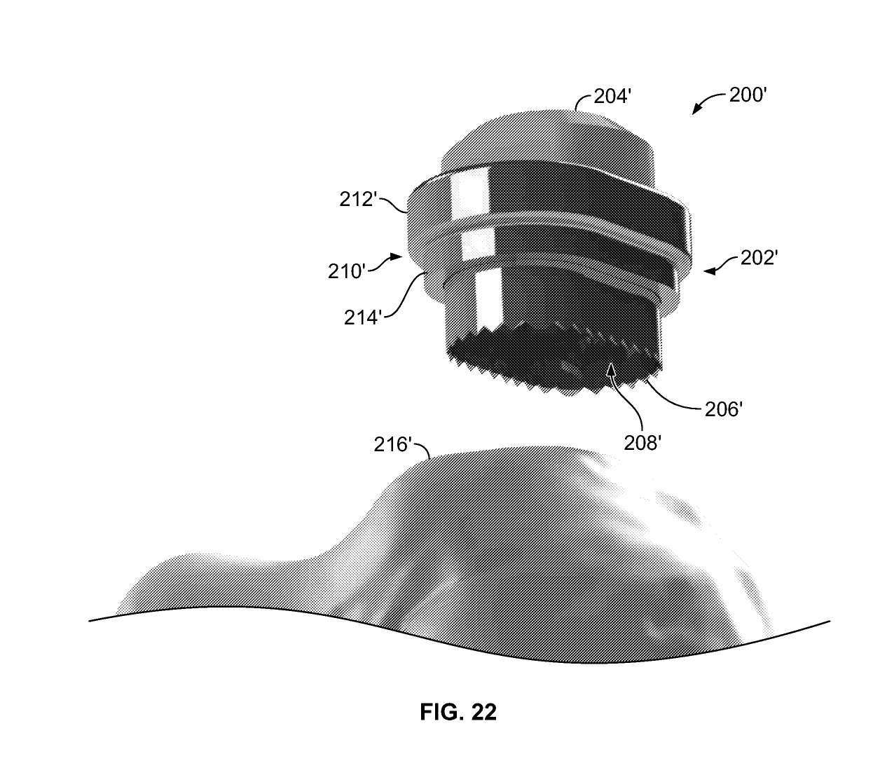

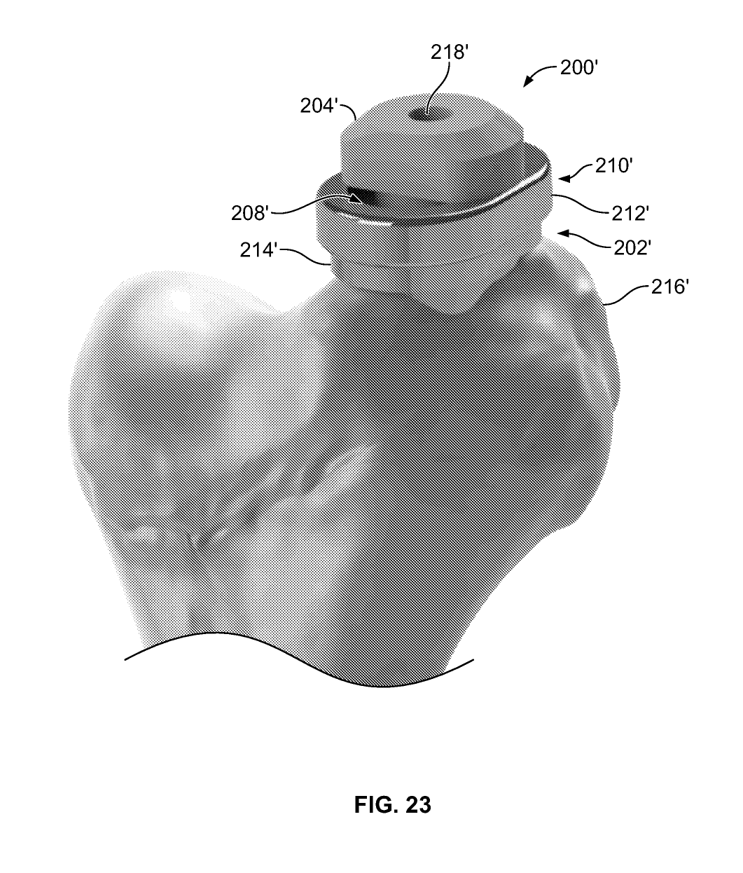

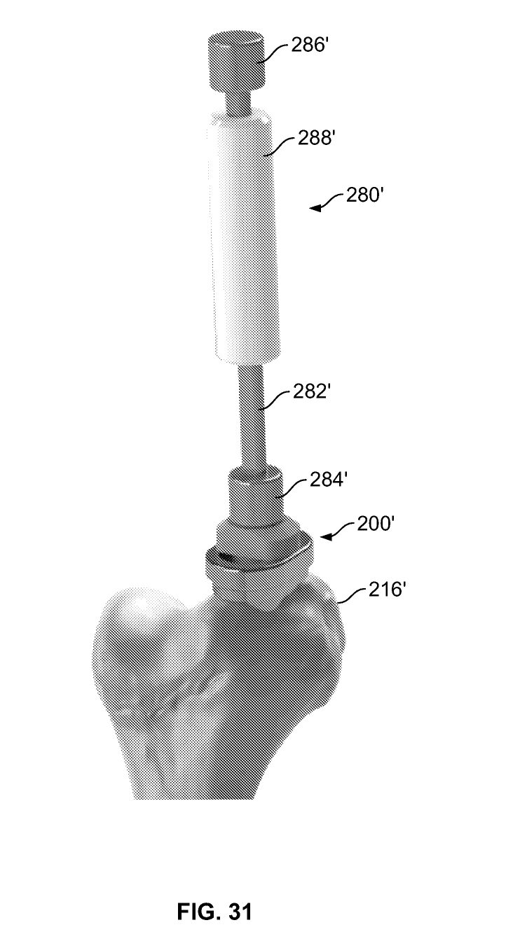

The assembly 200′ can generally be adapted to cooperate with an ancillary device (see, e.g., FIG. 31), e.g., a slap hammer, that facilitates driving the punch 202′ into a desired anatomical location. Detachable coupling of the ancillary device relative to the guide 204′ can be accomplished in various ways, e.g., threading of a distal portion of the ancillary device into a threaded aperture 218′ formed in guide 204′. In some embodiments, rather than or in combination with being driven into the anatomical location, the punch 202′ can be secured or fixated to the anatomical location with, e.g., screws, K-wires, and the like. FIG. 22 shows a bottom perspective view of the exemplary assembly 200′. As illustrated in FIG. 23, the assembly 200′ can be positioned above and driven into a desired anatomical location around a defect. Once the assembly 200′, i.e., the cutter assembly, has been driven to a desired depth or the depth allowed by the second peripheral protrusion 214′, the ancillary device can be detached from the guide 204′. The guide 204′ can generally be detached from the punch 202′ by rotating the guide 204′ and unlocking the locking features 220′ from the complementary slots 224′, as illustrated in FIG. 24.

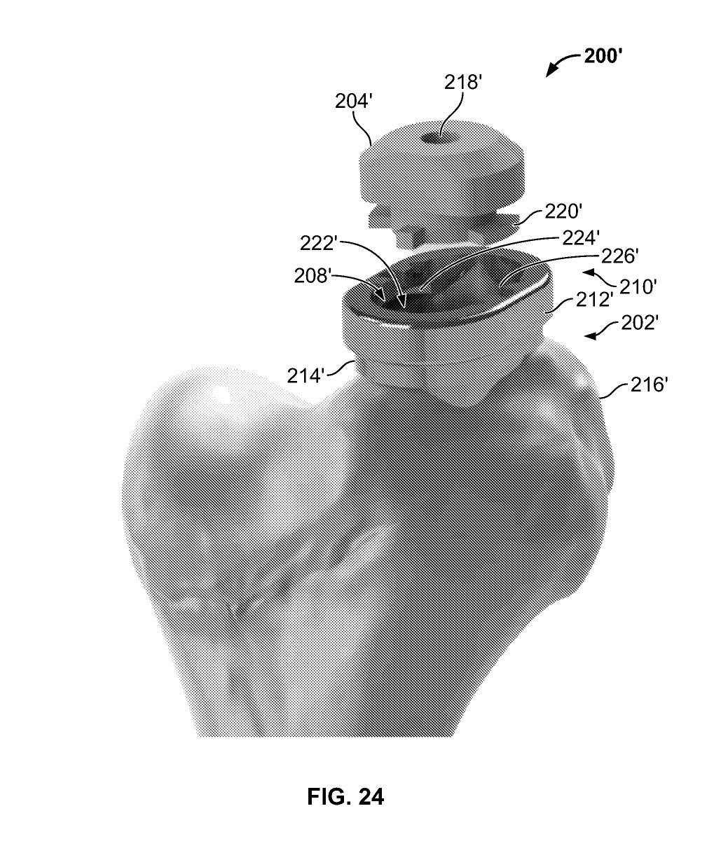

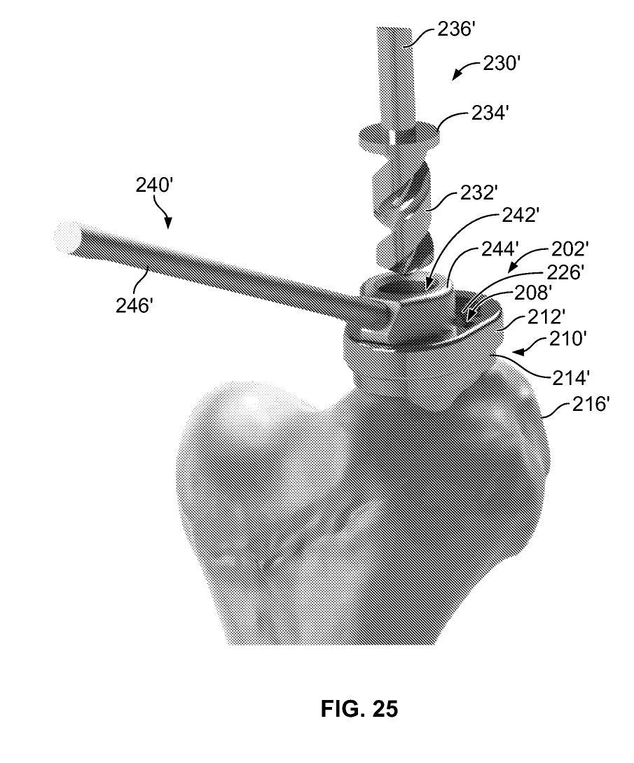

Turning to FIG. 25, once the punch 202′ has been driven to a desired depth in the anatomical location and the ancillary device and the guide 204′ have been decoupled and removed from the punch 202′, the surgeon can introduce a cutting member 230′ through a cutting aperture 242′ defined by an inner periphery of the cutting member guide 240′, i.e., a guide member. In some embodiments, the cutting member guide 240′ can include a guide section 244′ and a handle 246′. The guide section 244′ includes the cutting aperture 242′ passing therethrough configured and dimensioned to receive the cutting member 230′. The cutting member guide 240′ can be interlocked relative to the punch 202′ by, e.g., locking means similar to that of the guide 204′. The cutting member 230′ generally includes a cutting bit 232′ that can be sized to closely cooperate with the cutting aperture 242′. In addition, the cutting member 230′ can include a stop 234′ that, through engagement with the top face of first peripheral protrusion 212′ of the punch 202′, controls the depth to which the cutting bit 232′ is permitted to cut into the cartilage 216′. The cutting member 230′ further includes a shaft 236′ secured to the stop 234′ configured and dimensioned to be mechanically interlocked relative to a drive mechanism (not shown) for driving the cutting member 230′ into the cartilage 216′.

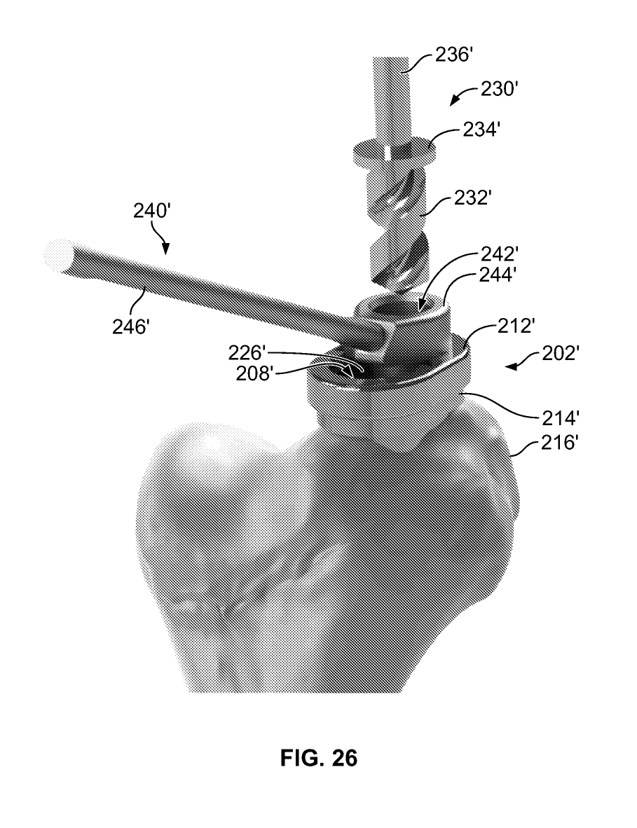

With reference to FIGS. 25 and 26, similar to the two-step drilling described above with respect to assembly 200, after a desired “first cut” to remove bulk material from the defect region has been completed, the cutting member 230′ can be separated from the cutting member guide 240′ and the cutting member guide 240′ can be removed from engagement with the punch 202′. The cutting member guide 240′ can then be rotated substantially 180° relative to the punch 202′ and recoupled thereto. In this way, the cutting aperture 242′ can be repositioned relative to the anatomical location for a “second cut” to remove bulk material from the defect region. As shown in FIG. 26, the cutting member 230′ can be reintroduced into cutting aperture 242′ and a second cut or drilling hole can be made to a desired depth. In this way, additional bulk material can be removed from the anatomical location containing the defect region.

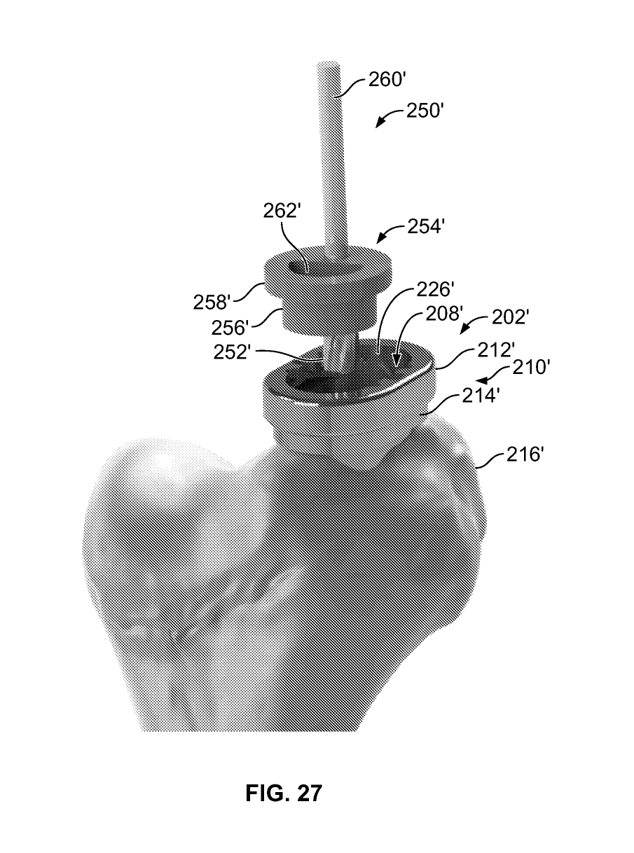

With reference to FIGS. 27-29, to clean-up the cuts made by the cutting member 230′, i.e., the “coarse” cuts to remove bulk material, a clean-up cutting step can generally be undertaken. Thus, in exemplary embodiments, the cutting member guide 240′ can be removed from the punch 202′ and a clean-up cutter 250′ can be introduced to the cutting region 208′ defined by the inner side wall 226′ of the punch 202′. The exemplary clean-up cutter 250′, i.e., a finishing cutter, generally includes a cutting bit 252′ and an offset bushing 254′ that guides travel of the clean-up cutter 250′ relative to the punch 202′. In particular, bushing 254′ generally includes a lower portion 256′ that engages the inner surface of side wall 226′ as the clean-up cutter 250′ travels within the cutting region 208′. The bushing 254′ generally includes an upper portion 258′ which rides along the top of side wall 226′ and the first peripheral protrusion 212′ and controls the depth to which the cutting bit 252′ engages the anatomical location. In some embodiments, the upper portion 258′ of the bushing 254′ can be configured and dimensioned to act as a visual indicator (similar to those discussed above) which, when aligned relative to an outer side wall of the first peripheral protrusion 212′ of the punch 202′, can indicate to the user that the cutting bit 252′ has been positioned substantially adjacent to the inner side wall 226′ of the punch 202′. The two portions of the bushing 254′ thereby form a stepped bushing 254′. The clean-up cutter 250′ further includes a shaft 260′ secured to the bushing 254′ configured and dimensioned to be mechanically engaged relative to a drive mechanism (not shown) to drive the clean-up cutter 250′ into the cartilage 216′. In some embodiments, the bushing 254′ can include an aperture 262′ passing therethrough to provide a means for a user to view the formation of the defect region cavity.

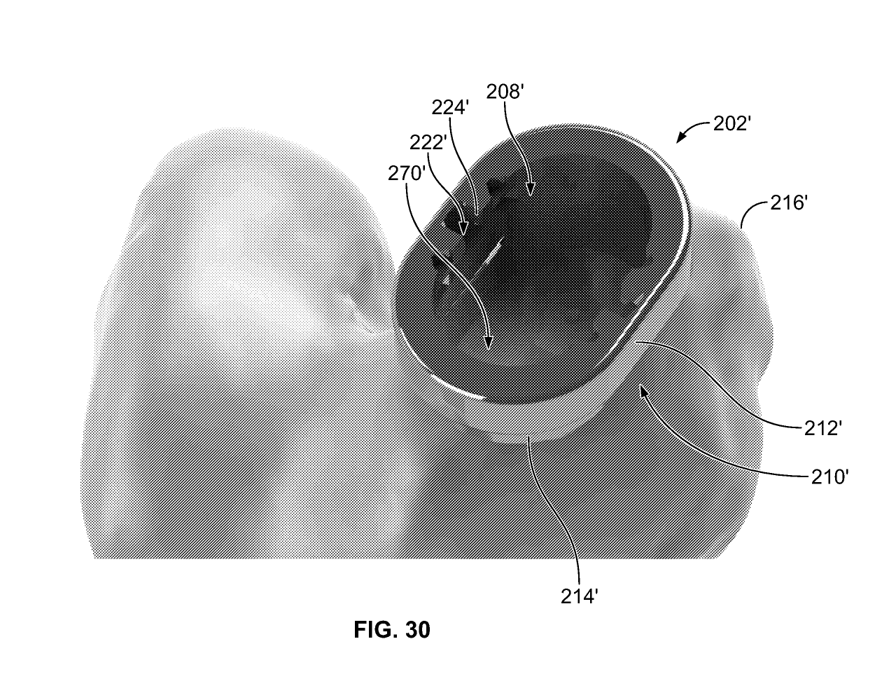

The clean-up cutter 250′ advantageously functions to remove material left in cutting region 208′ by the first and second cutting actions of the cutting member 230′, thereby generating a substantially clean and uniform cut to a desired depth with a peripheral geometry substantially defined by the side wall 226′ of punch 202′. For example, the clean-up cutter 250′ can create a substantially flat bottom surface of the defect region cavity configured and dimensioned to receive a complementary implant graft. In general, a single instance of travel of the clean-up cutter 250′ within the cutting region 208′ can be effective to achieve the desired result, i.e., a finished cut forming a defect region cavity 270′ as shown in FIG. 30, although multiple travel instances may be undertaken, as desired by the surgeon.

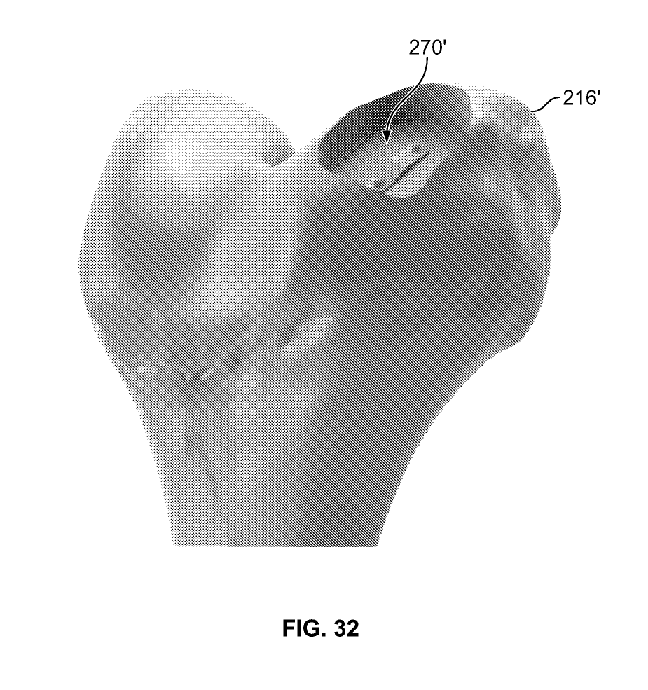

With reference to FIGS. 31 and 32, after completion of the clean-up cut, the guide 204′ can be reattached to the punch 202′ and the ancillary device 280′ can be reengaged with the associated aperture 218′ of the guide 204′ to facilitate removal (without twisting) of the assembly 200′ from the anatomical site. The ancillary device 280′, e.g., a slap hammer, generally includes an elongated shaft 282′, a guide attachment 284′, a top cap 286′ and a hammer mechanism 288′. The guide attachment 284′ can be configured and dimensioned to interlock relative to the guide 204′ via, e.g., a threaded shaft extending from the guide attachment 284′ for threading into the aperture 218′ of the guide 204′. The hammer mechanism 288′ can be slidably engaged relative to the shaft 282′ to allow a user to slide and create a force against the guide attachment 284′ and/or the top cap 286′ for generating a driving force relative to the assembly 200′. For example, the hammer mechanism 288′ can be hammered against the guide attachment 284′ to drive the assembly 200′ into the cartilage 216′ and can be hammered against the top cap 286′ to remove the assembly 200′ from the cartilage 216′. As is apparent from FIG. 32, the defect region cavity 270, i.e., an implant region, formed at the anatomical site can be substantially clean and uniform in both peripheral geometry and depth.

With reference now to FIG. 33, an exemplary template assembly 200″ is provided, generally including a punch 202″, i.e., a template, and a guide 204″. The punch 202″ generally includes a cutting edge 206″ around an exposed periphery of the punch 202″. The cutting edge 206″ can define a “clean” cutting blade (not shown), a serrated blade, or combinations/variations thereof. As discussed above, the cutting edge 206″ defines the preformed geometric shape opening for forming the defect region cavity and may define, e.g., a racetrack, oval, pear-shaped, circular, rectangular, and the like, periphery. The punch 202″ and the guide 204″ generally define a cooperating interlocking mechanism, e.g., a keying feature, which guides interaction between the punch 202″ and the guide 204″. The guide 204″ can be inserted into the aperture 208″ of the punch 202″ and, in particular, the locking features or protrusions (not shown) of the guide 204″ can be inserted through complementary slots 210″ in a top surface 212″ of the punch 202″. The guide 204″ can further be interlocked with the punch 202″ by rotating the guide 204″ and, in turn, the locking features or protrusions of the guide 204″, within a path or groove on the inside of the punch 202″. The detachable guide 204″ thereby creates a partial extent of coverage relative to the punch 202″, i.e., the detachable guide 204″ essentially operates within a portion of the punch 202″ rather than engaging the periphery of the punch 202″.

The template assembly 200″ can generally be adapted to cooperate with an ancillary device, e.g., a slap hammer, a crank-actuated mechanism, and the like, that facilitates driving the punch 202″ into a desired anatomical location. For example, in some exemplary embodiments, the slap hammer can be used to drive the punch 202″ into the cartilage 214″. In some exemplary embodiments, a crank-actuated mechanism can be used to at least partially drive the punch 202″ into the cartilage 214″. As described above, the crank-actuated mechanism may be used to, e.g., reduce the toggle effect of the slap hammer, accurately drive the punch 202″ into the cartilage 214″ based on the desired angle and/or orientation, and the like. Detachable coupling of an ancillary device relative to the guide 204″ can be accomplished in various ways, e.g., threading of a distal portion of the device into a threaded aperture 216″ formed in the guide 204″, and the like. In some embodiments, rather than or in combination with being driven into the anatomical location, the punch 202″ can be secured or fixated to the anatomical location with, e.g., screws, K-wires, and the like.

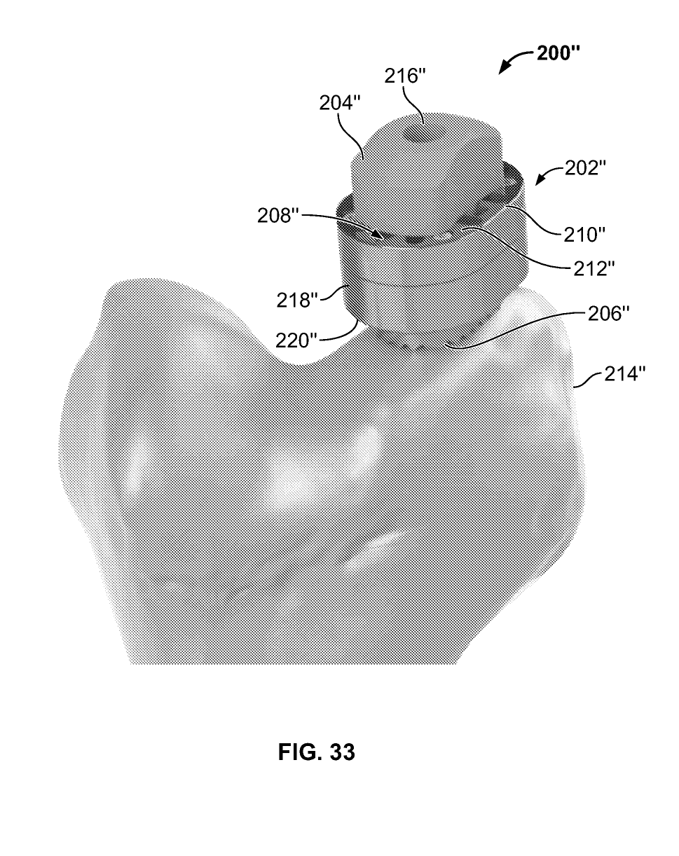

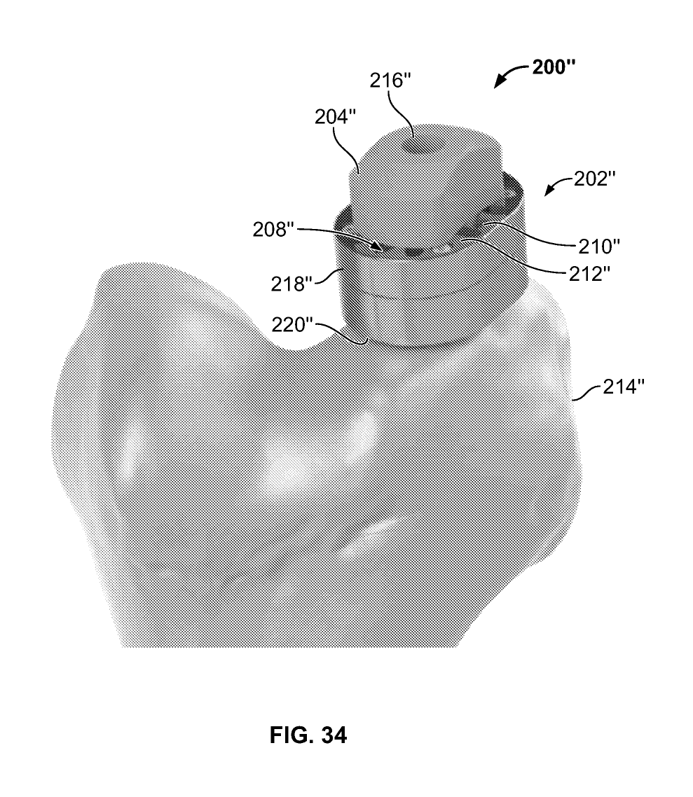

As illustrated in FIG. 33, the template assembly 200″ can be positioned on and driven into a desired anatomical location, e.g., cartilage 214″, around a defect. A bottom surface 220″ of a punch stop element 218″, e.g., a protrusion around the periphery of the punch 202″, and/or the length of the cutting edge 206″ protruding from the punch 202″ can be used to regulate the depth to which the punch 202″ is driven into the cartilage 214″. Once the template assembly 200″ has been driven into the cartilage 214″ to a desired depth (as shown in FIG. 34), the ancillary device can be detached from the guide 204″ and the guide 204″ can be detached from the punch 202″ by rotating and unlocking the locking features of the guide 204″ and the complementary slots of the punch 202″.

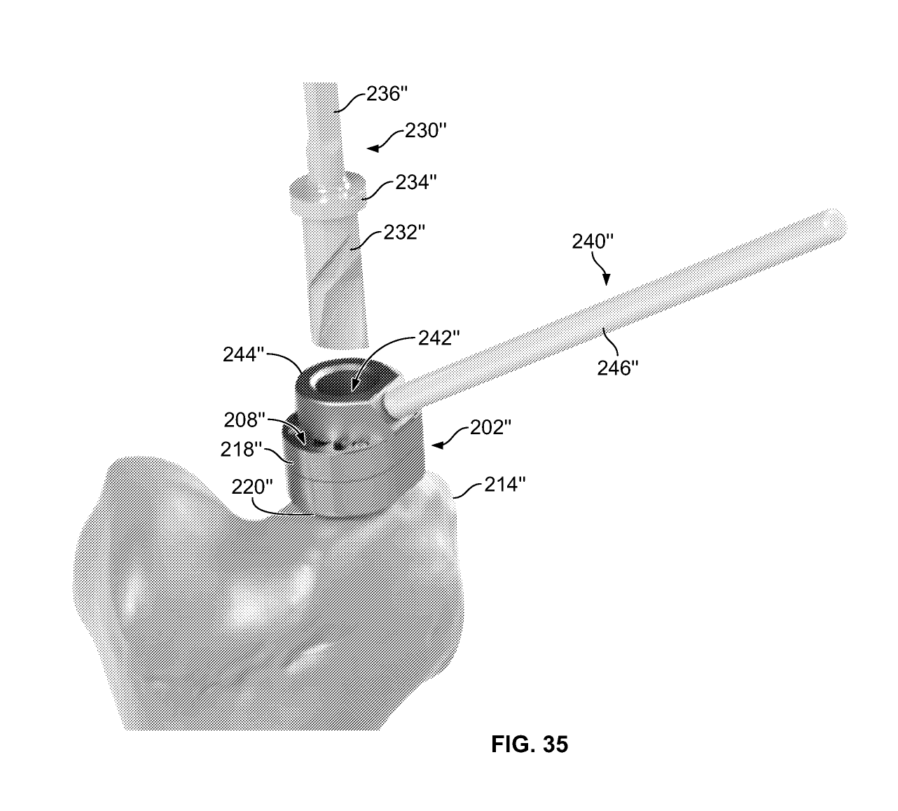

Turning now to FIG. 35, once the punch 202″ has been driven to a desired depth in the cartilage 214″ and the ancillary device and guide 204″ have been decoupled and removed from the punch 202″, the surgeon can introduce a cutting member 230″ through a cutting aperture 242″ defined by the inner periphery of the cutting member guide 240″, i.e., a guide member. In some embodiments, the cutting member guide 240″ can include a guide section 244″ and a handle 246″. The guide section 244″ includes the cutting aperture 242″ passing therethrough configured and dimensioned to receive the cutting member 230″. The cutting member 230″ can be implemented for removal of “bulk” material from the anatomical site. The cutting member guide 230″ can be interlocked relative to the punch 202″ by, e.g., locking means substantially similar to that of the guide 204″. The cutting member 230″ generally includes a cutting bit 232″, i.e., a drill bit, that can be sized to closely cooperate with the cutting aperture 242″. In addition, the cutting member 230″ can include a stop element 234″ that, through engagement with the top surface of the punch stop element 218″ or the top face of the guide section 244″ of the cutting member guide 240″, controls the depth to which the cutting bit 232″ will cut into the cartilage 214″. The cutting member 230″ further includes a shaft 236″ secured to and axially extending from the stop 234″ configured and dimensioned to be mechanically interlocked relative to a drive mechanism (not shown) for driving the cutting member 230″ into the cartilage 214″. Although illustrated as a cutting member 230″ and a cutting member guide 240″, in some exemplary embodiments, the alternative cutters discussed herein may be implemented to create the defect region cavity and the depth of the defect region cavity can be regulated by the position of the stop element 234″ relative to the top surface of the punch stop element 218″.

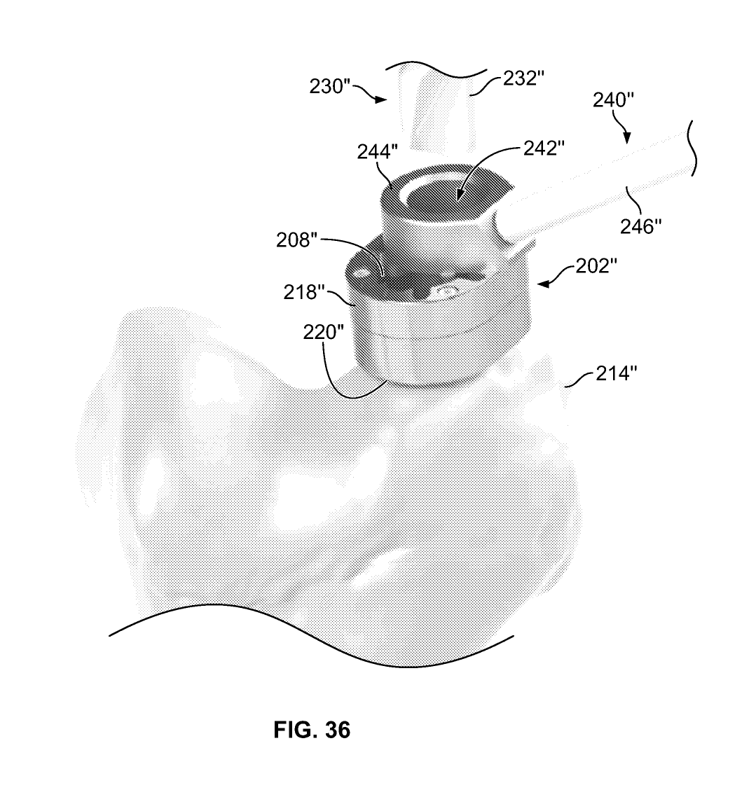

With reference to FIG. 36, after a desired “first cut” has been completed, the cutting member 230″ can be separated from the cutting member guide 240″ and the cutting member guide 240″ can be removed from engagement with the punch 202″. The cutting member guide 240″ can then be rotated substantially 180° relative to the punch 202″ and then recoupled thereto. Thus, the cutting aperture 242″ can be repositioned relative to the anatomical location for a “second cut”. In this way, additional bulk material can be removed from the anatomical location to create the defect region cavity.

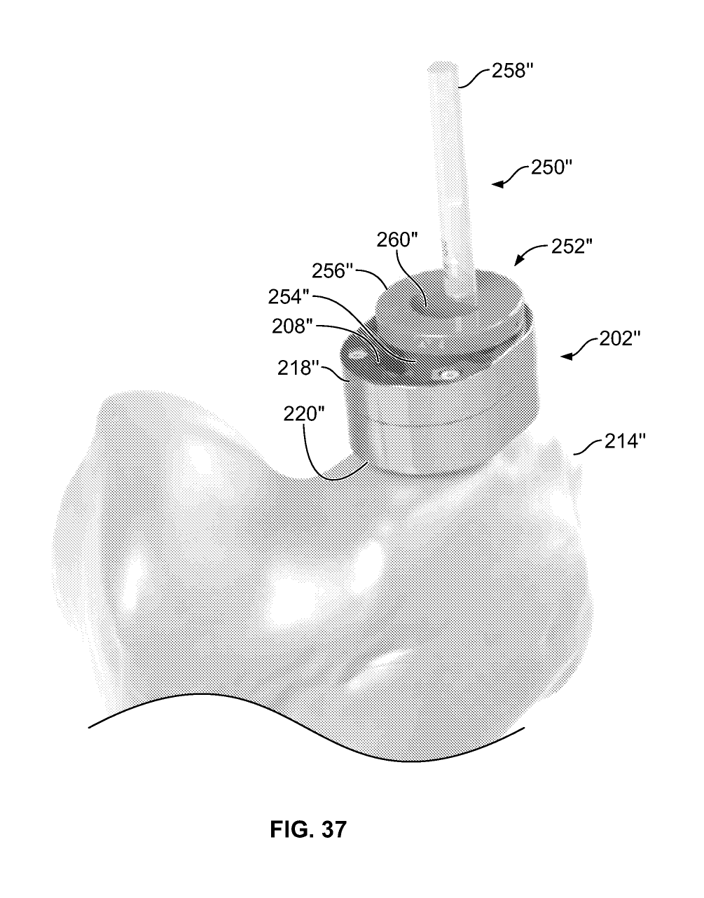

With reference to FIG. 37, to clean up the cuts made by cutting member 230″, i.e., the “coarse” cuts for removing bulk material, a clean-up cutting step can be undertaken. In exemplary embodiments, the cutting member guide 240″ can be removed from the punch 202″ and a clean-up cutter 250″ can be introduced to the cutting region defined by the inner side walls of the aperture 208″ of the punch 202″. The exemplary clean-up cutter 250″, i.e., a finishing cutter, generally includes a cutting bit (not shown) and an offset bushing 252″ that guides travel of the clean-up cutter 250″ relative to the punch 202″. In particular, bushing 252″ includes a lower portion 254″ that engages the inner surface of the side walls of the aperture 208″ of the punch 202″ as the clean-up cutter 250″ travels within the cutting region, i.e., the region defined by the inner side walls of the aperture 208″. The bushing 252″ further includes an upper portion 256″ that rides along the top surface of the punch stop element 218″ and controls the depth to which the cutting bit engages the cartilage 214″. In some embodiments, an alignment of the side of the upper portion 256″ relative to the side of the punch stop element 218″ can act as a visual indicator to a user, e.g., a surgeon, to indicate that the cutting bit passing within the aperture 208″ is substantially aligned with the inner side walls of the punch 202″. In some embodiments, the bushing 252″ can include an aperture 260″ passing therethrough to provide a means for a user to view the formation of the defect region cavity. For example, the shaft 258″ and the aperture 260″ can be offset relative to the central axis of the bushing 252″ on opposing sides of the busing 252″. In some embodiments, the aperture 260″ can be configured as a semi-circle.

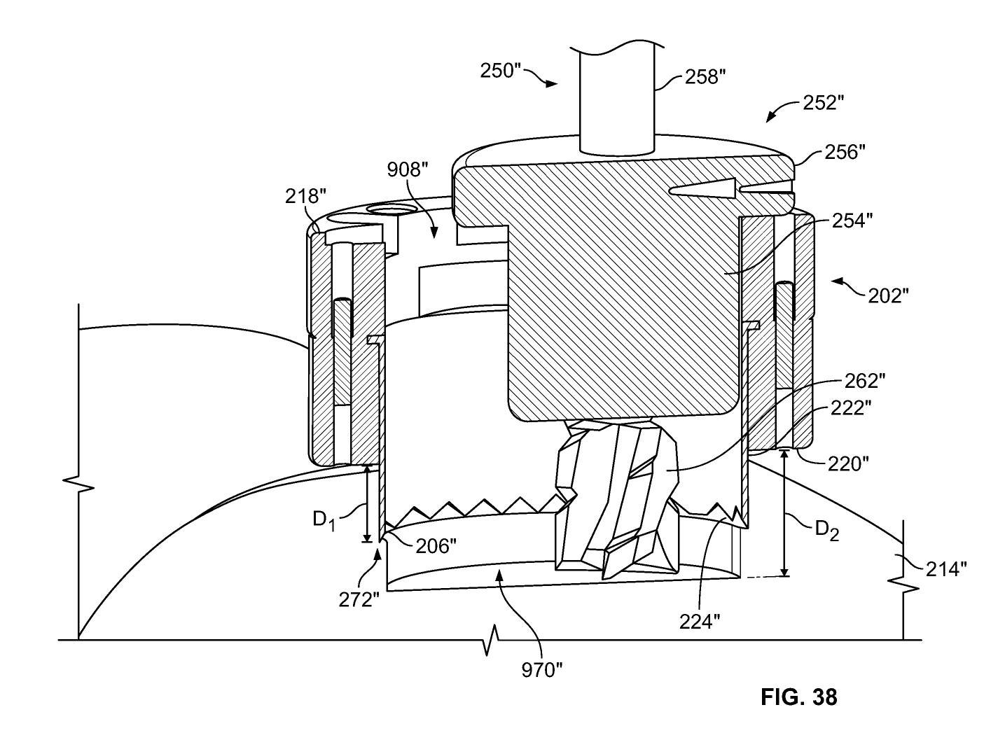

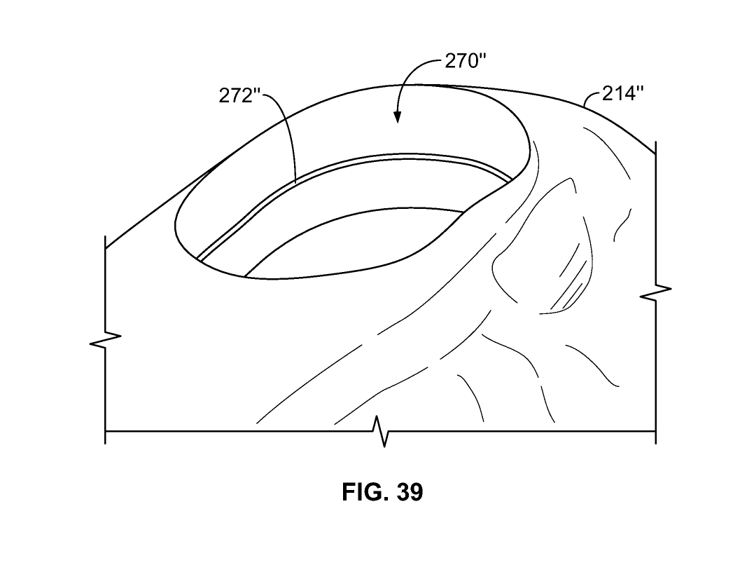

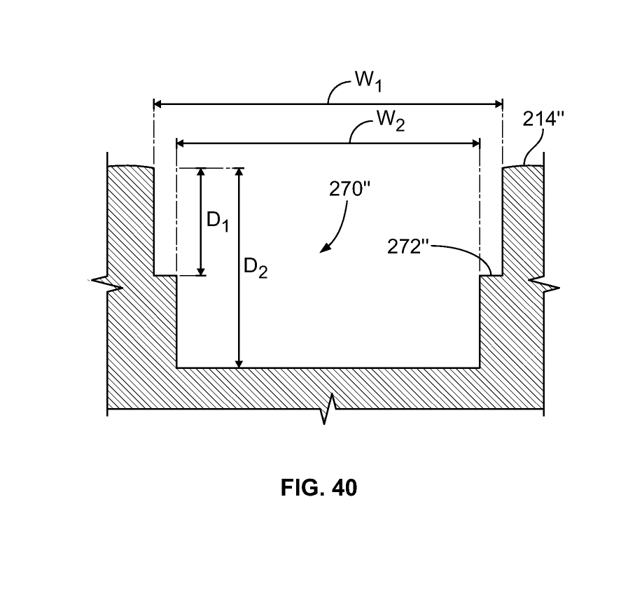

The clean-up cutter 250″ advantageously functions to remove material left in the cutting region by the first and second cutting actions of cutting member 230″, thereby generating a substantially clean and uniform cut to a desired depth with a peripheral geometry substantially defined by the side walls of the aperture 208″ of the punch 202″. A single instance of travel of the clean-up cutter 250″ within the cutting region can generally be effective to achieve the desired result, i.e., a finished cut as shown in FIGS. 39 and 40, although multiple travel instances may be undertaken, as desired by the surgeon. After completion of the clean-up cut, the guide 204″ can be reattached to the punch 202″ and the ancillary device (or an alternative device) can be reengaged with the associated aperture 216″ to facilitate removal (without twisting) of the assembly 200″ from the anatomical site. For example, if implementing a slap hammer, the hammer mechanism can be driven or hammered against the top cap of the slap hammer to drive the assembly 200″ vertically out of the anatomical site without twisting. Thus, the implant region or defect region cavity formed at the anatomical site can be substantially clean and uniform in both peripheral geometry and depth.

In some exemplary embodiments, the template assembly 200″ can be implemented for creating a “stepped” defect cavity region, i.e., implant region, for creating a press fit with a donor plug. With reference to FIG. 38, a cross-sectional view of the punch 202″ and clean-up cutter 250″ is illustrated for forming the “stepped” defect cavity region. However, it should be understood that the bulk cutting member 230″ can be implemented in the same manner as discussed herein with respect to the clean-up cutter 250″. In particular, the cutting edge 206″ of the blade of the punch 202″ can initially be driven into the cartilage 214″ until the bottom surface 220″ of the punch stop element 218″ abuts the top surface of the cartilage 214″ and the punch 202″ cannot be driven deeper. For example, the punch depth D₁ can be approximately 6 mm into the cartilage 214″. The width, e.g., the diameter, and the like, of the cut created by the blade of the punch 202″ can be defined by the width of the outer surface 222″ of the blade. In particular, as the blade of the punch 202″ is driven into the cartilage 214″, the cut formed can be substantially equivalent in width to the width of the outer surface 222″ of the blade.

When the drill bit 262″ of the clean-up cutter 950″ (or the cutting bit 232″ of the cutting member 230″) is utilized to remove the cartilage 214″ from the defect region, the drill bit depth D₂ can be regulated such that the drill bit depth D₂ is greater than the punch depth D₁. For example, if the punch depth D₁ is approximately 6 mm, the drill bit depth D₂ can be approximately 10 mm. However, it should be understood that the difference between the punch depth D₁ and the drill bit depth D₂ can vary depending on the size of the press fit desired. In addition, as described above, the drill bit 262″ position relative to the aperture 908″ defining the inner side surfaces of the punch 202″ can be regulated by, e.g., bushing 252″. Thus, the drill bit 262″ position can be regulated to remove cartilage material to form a defect region cavity 970″ having a width slightly smaller than the width formed by the blade of the punch 202″. In particular, as the drill bit 262″ moves within the aperture 908″ of the punch 202″, the cut formed can be substantially equivalent to the width of the inner surface 224″ of the blade. A step 272″ can thereby be formed between the cut created by the blade of the punch 202″ and the deeper cut formed by the drill bit 262″. Although illustrated in FIG. 38 as being dimensioned substantially equivalent to the width of the blade of the punch 202″, in some exemplary embodiments, the step 272″ width can be regulated to be greater or smaller than the width of the blade of the punch 202″ to vary the force imparted by the press fit onto the donor plug implanted within the defect region cavity 270″.

With reference to FIG. 39, an exemplary “stepped” defect region cavity 270″ formed in cartilage 214″ is illustrated after the punch 202″ has been removed. In particular, the defect region cavity 270″ defines a step 272″ for creating a press fit onto the donor plug implanted within the defect region cavity 270″. FIG. 40 is a cross-sectional view of an exemplary “stepped” defect region cavity 270″. As described above, the difference in punch depth D₁ and drill bit depth D₂ can be regulated by, e.g., bushing 252″. The point at which the depth changes between the punch depth D₁ and the deeper drill bit depth D₂ can be the location of the step 272″. As also described above, the difference in widths, i.e., between the punch width W₁ and the drill bit width W₂, can be varied to change the force imparted by the press fit onto the donor plug implanted within the defect region cavity 270″.

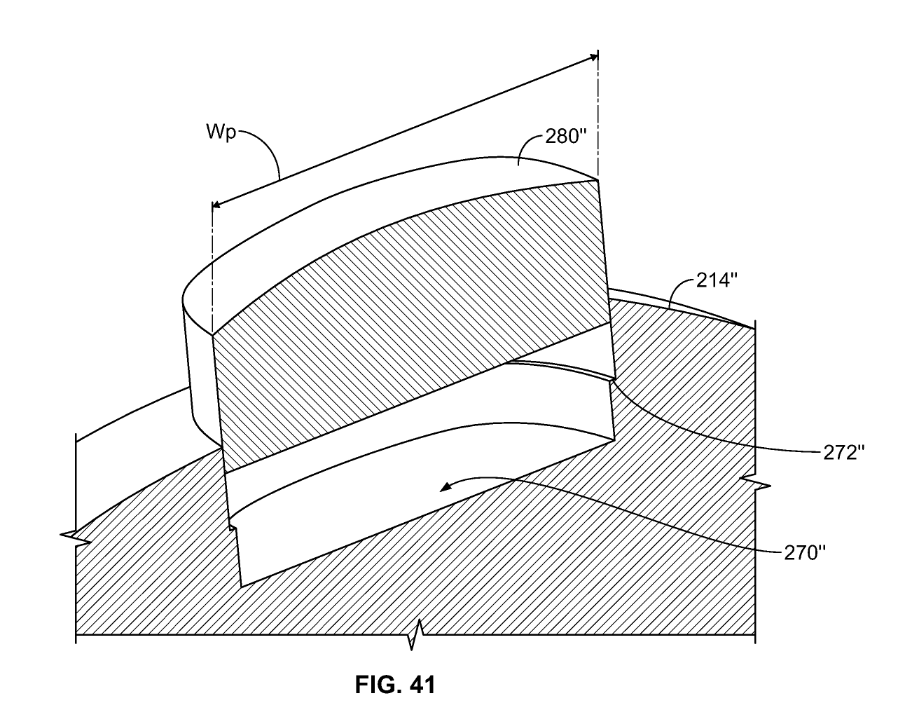

Turning now to FIG. 41, an exemplary donor plug 280″ is shown being placed in the “stepped” defect region cavity 270″. The plug width W_{P} can be dimensioned substantially similarly to the punch width W₁ such that the donor plug 280″ initially fits into the defect region cavity 270″ easily until it reaches the step 272″. In particular, the graft harvesting devices discussed herein can be utilized to property size the plug width W_{P} such that the donor plug 280″ initially easily fits within the defect region cavity 270″. Once the donor plug 280″ reaches the step 272″, the donor plug 280″ can be fully pressed into the defect region cavity 270″ by imparting a greater force onto the donor plug 280″. A press fit can thereby be created between the defect region cavity 270″ and the donor plug 280″. It should be understood that the press fit generally enhances the stability and/or strength of the implanted donor plug 280″ within the defect region cavity 270″. FIG. 42 illustrates the donor plug 280″ fully inserted into the “stepped” defect region cavity 270″. As can be seen from FIG. 42, the surface topography of the donor plug 280″ substantially matches the surface topography of the area surrounding the defect region cavity 270″. In particular, a substantially line-to-line fit is created at the surface of the cartilage 214″ and the donor plug 280″ to maintain precise contour matching.

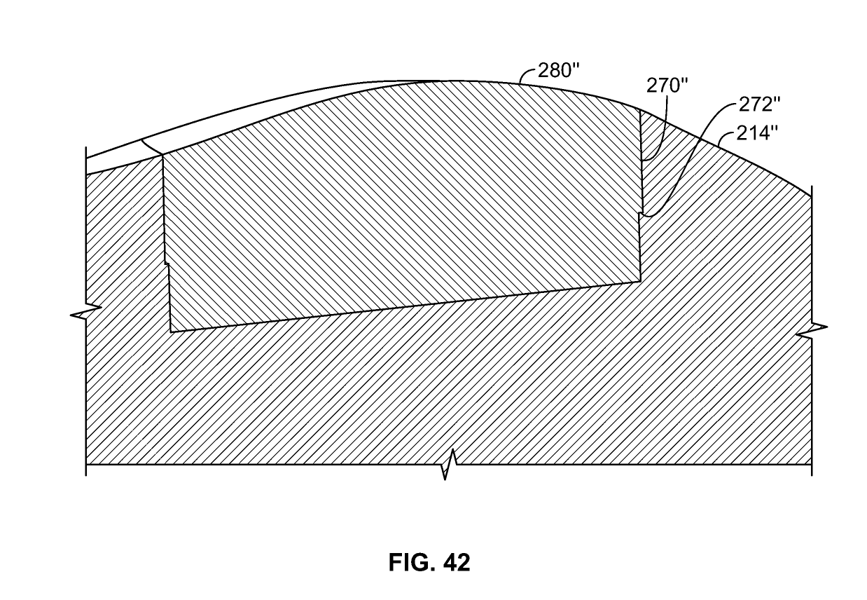

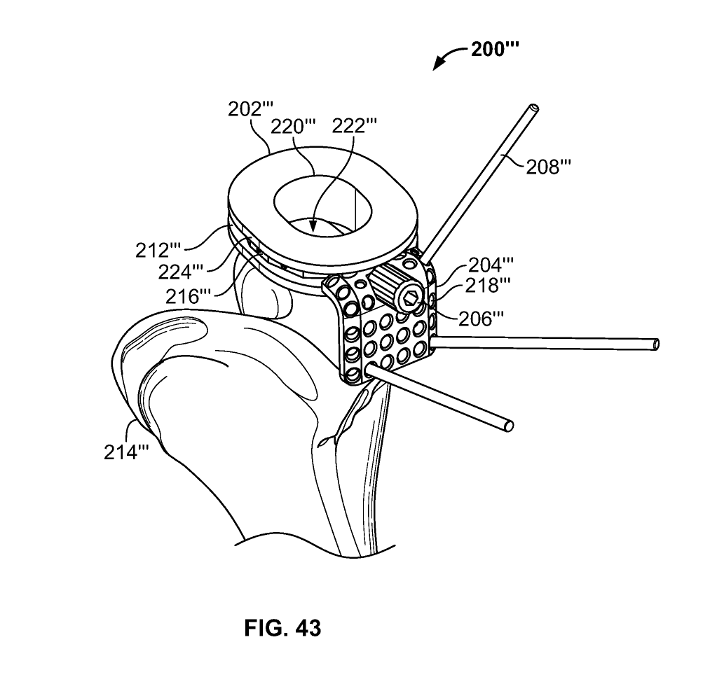

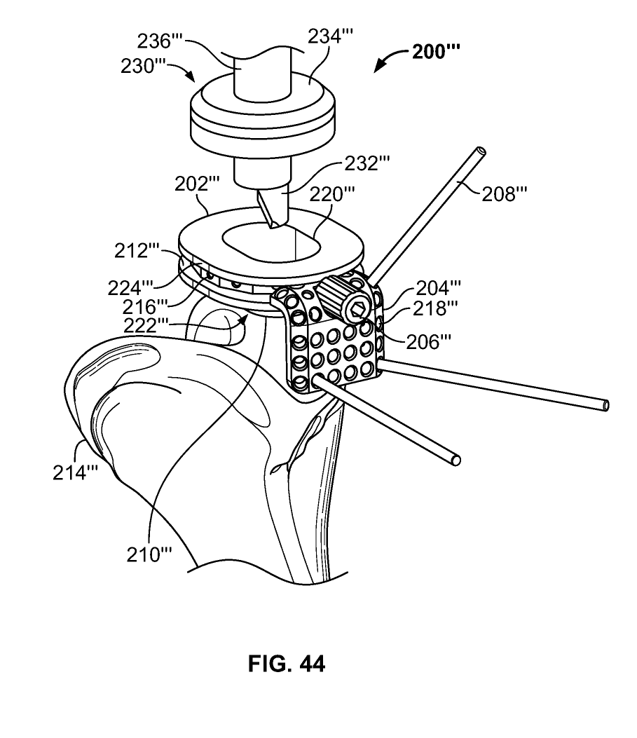

Turning now to FIG. 43, an exemplary embodiment of a template assembly 200′″ is depicted according to the present disclosure, generally including an exemplary template 202′″ (e.g., a punch), a mounting track 204′″, a locking screw 206′″, and a plurality of K-wires 208′″. Initially, as shown in FIG. 43, the template bottom portion 210′″ (see, e.g., FIG. 44) of the template 202′″ can be driven into the cartilage 214′″ with an ancillary device, e.g., a slap hammer, a crank-actuated mechanism, and the like. In some embodiments, rather than or in combination with being driven into the anatomical location, the template 202″ can be secured to the cartilage 214″ with, e.g., screws, K-wires, and the like. In some embodiments, the template bottom portion 210′″ can be configured as a serrated or non-serrated blade. As can be seen from FIG. 43, the template 202′″ generally includes a peripheral template track 212′″, e.g., a recessed portion between the top and bottom surfaces of the template 202′″ for partially receiving placement of the mounting track 204′″ and anchoring the template 202′″ relative to an anatomical structure, e.g., cartilage 214′″ of a knee. In general, the template 202′″ also includes a plurality of pre-drilled holes 216′″ configured to receive and/or mate with the locking screw 206′″ (or a plurality of locking screws; not pictured). In addition to anchoring of the template 202′″ via the template bottom portion 210′″ driven into the cartilage 214′″, the peripheral template track 212′″ provides flexibility to a user for anchoring the template 202′″ relative to an anatomical substrate by permitting approximately 360° of mounting access. The peripheral template track 212′″ also ensures that the components of the template assembly 200′″ are properly oriented or aligned relative to a desired cavity region prior to forming the defect region cavity.

The mounting track 204′″ can be manufactured from a flexible yet durable material, e.g., rubber, and can be detachably secured relative to the template 202′″ by inserting a portion of the mounting track 204′″ into the groove formed in the peripheral template track 212′″ of the template 202′″ and inserting the locking screw 206′″, e.g., a set screw, thumb screw, and the like, into an appropriate pre-drilled hole 216′″. The mounting track 204′″ can thereby be detachably secured between the template 202′″ and the locking screw 206′″. Interaction between the locking screw 206′″ and a plurality of circumferentially spaced holes 216′″ permits the mounting track 204′″ to be detachably secured to the template 202′″ at a variety of orientations and can be secured to a pre-drilled hole 216′″ by hand, thereby reducing the number of tools required for surgery. The mounting track 204′″ further includes a plurality of rows/columns of K-wire holes 218′″ for insertion of K-wires 208′″ in order to secure the template 202′″ relative to the cartilage 214′″ during use. In particular, the plurality of rows/columns of K-wire holes 218′″ can be oriented at varying angles relative to a mounting surface, thereby permitting the plurality of K-wires 208′″ to be inserted at varying angles for a more secure attachment to a desired anatomical location, e.g., to prevent motion of the template 202′″ during use.

The template 202′″ generally defines a geometric shape opening 220′″ configured and dimensioned to surround a defect region 222′″ in the cartilage 214′″. Although illustrated as oval in shape, in some embodiments, the geometric shape opening 220′″ can define, e.g., a circular shape, a square shape, a rectangular shape, an irregular shape, and the like, and can be dimensioned in various sizes to effectively fully surround the defect region 222′″. In some embodiments, the template 202′″ can be configured to allow a user, e.g., a surgeon, to vary the configuration of the geometric shape opening 220′″ to conform and customize the template 202′″ to the defect region 222′″ configuration and/or dimensions. The template 202′″ also defines a template side surface 224′″. The template side surface 224′″ can define a peripheral projection which protrudes wider than the template bottom portion 210′″. Thus, in some embodiments, the peripheral template track 212′″ can act as a stop element to control the depth to which the template bottom portion 210′″ can be driven into the cartilage 214′″.

Turning now to FIG. 44, the exemplary template assembly 200′″ is illustrated, including an exemplary cutter 230′″ approaching the template 202′″. In particular, FIG. 44 depicts the cutter 230′″ being lowered in the direction of the preformed geometric shape opening 220′″ of the template assembly 200′″ in preparation for forming a defect region cavity. The cutter 230′″ can be substantially similar to the cutters described above, generally including a drill bit 232′″, a stop element 234′″, e.g., a bushing, and a shaft 236′″ for mechanically connecting the cutter 230′″ to a drive mechanism (not shown). As discussed above, the drill bit 230′″ can be, e.g., a downcutting drill bit as described in a U.S. patent application entitled “Orthopedic Downcutting Instrument and Associated Systems and Methods,” which published as US 2011/0238070 A1, and was previously incorporated herein by reference.

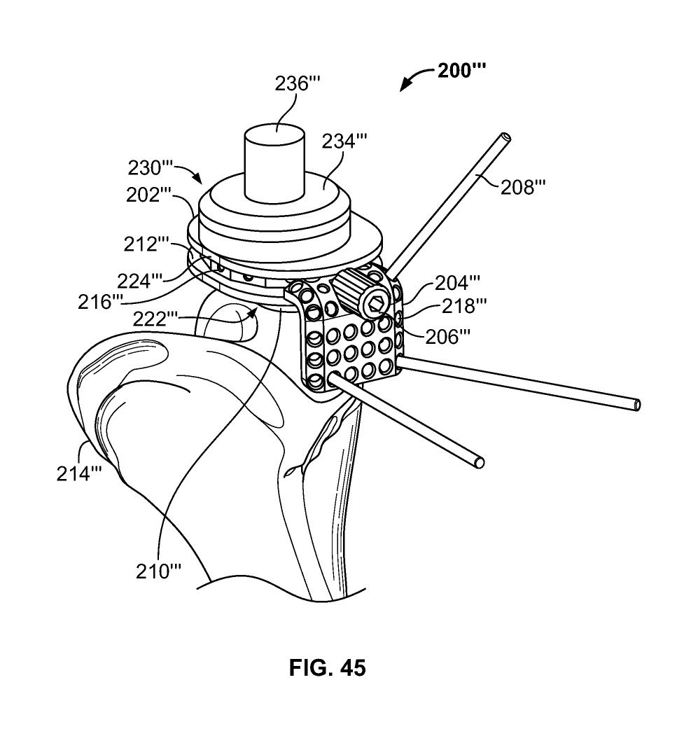

With reference to FIG. 45, the drill bit 232′″ of the cutter 230′″ has been lowered/inserted into the preformed geometric shape opening 220′″ of the template 202′″ to form the defect region cavity in the cartilage 214′″. As discussed previously, an alignment of a side surface of the stop element 234′″ of the cutter 230′″ and a template side surface 224′″ can indicate to a user that the drill bit 232′″ has reached the edge, i.e., an inner side surface, of the preformed geometric shape opening 220′″ of the template 202′″. This visual indicator or reference can be utilized by a user for awareness of where a cut has been made in the cartilage 214′″ relative to the opening formed in the template 202′″. When the defect region cavity has been formed in the cartilage 214′″, the template 202′″ can be removed for performing the subsequent steps described below. Although the template 202′″ is used herein to form a defect region cavity defined by substantially parallel walls, as described above, in some exemplary embodiments, the template 202′″ may be used to form stepped region cavity walls for creating a press fit with a donor plug.

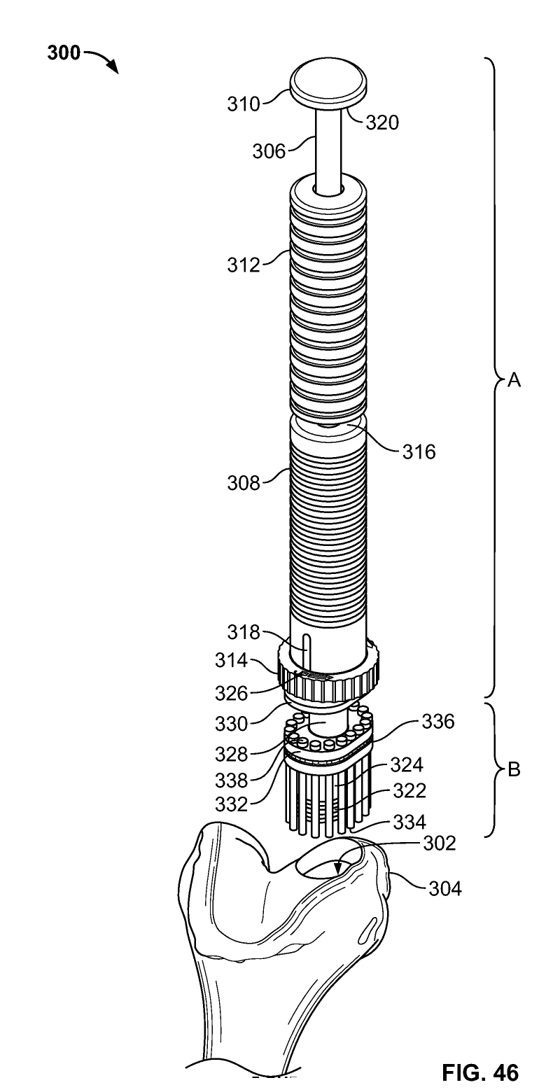

Turning to FIG. 46, an exemplary embodiment of a graft harvesting device 300 is provided, generally configured as a reusable portion A and a disposable portion B for harvesting a graft plug to fill a defect region cavity 302, e.g., a smooth defect region cavity, formed in the cartilage 304, and manufactured from medically acceptable materials, e.g., stainless steel. In particular, the disposable portion B can be detachably connected to the reusable portion A and can be replaced by a customized disposable portion B, e.g., alternatively configured and/or dimensioned disposable portion B depending on the size and/or shape of the defect region cavity 302 being operated on. On the other hand, the reusable portion A can generally be configured and dimensioned in a standard size to be functional in alternatively sized operations. The ability to reuse the reusable portion A generally lowers the costs and the number of required instruments associated with the disclosed procedure relative to those taught in the prior art. Of note, the present disclosure is not limited by or to the reusable/disposable modality described above. Rather, it may be advantageous to supply detachably coupling subassemblies A and B that are both reusable and/or both disposable without departing from the spirit or scope of the present disclosure.

Still with reference to FIG. 46, the reusable portion A generally includes an elongated shaft 306, a handle 308, a top cap 310, a hammer mechanism 312 and a broach flange 314. The elongated shaft 306 can extend the length of the reusable portion A, thereby connecting the top cap 310 and the handle 308 at opposite ends relative to each other. The handle 308 can be securely/fixedly attached to the elongated shaft 306 and can include surface features, e.g., ridges, and/or be manufactured from a material permitting a strong and/or comfortable grasp by a user, e.g., foam, rubber, and the like. In some embodiments, the handle 308 can be manufactured to define a substantially smooth outer surface. The top surface 316 of the handle 308 located adjacent to the hammer mechanism 312 can be manufactured from a more durable material to permit hammering thereon with the hammer mechanism 312. The handle 308 can include a broach flange path 318 for axial translation of the broach flange 314, which will be described in greater detail below.

The hammer mechanism 312, e.g., a slap hammer, can freely slide axially along the elongated shaft 306 between the top cap 310 and the handle 308. The axial translation of the hammer mechanism 312 can be utilized for hammering, e.g., forcibly driving and/or axially applying a force, to the graft harvesting device 300 in a downward direction by hammering against the top surface 316 of the handle 308 and/or in an upward direction by hammering against the bottom surface 320 of the top cap 310. Thus, the hammer mechanism 312 permits the surgeon to apply an axial force to advance and/or withdraw the components of the disposable portion B into and/or from the cartilage 304 without accessing an auxiliary force-delivering device and without twisting of the disposable portion B within the defect region cavity 302.

In some exemplary embodiments, rather than (or in combination with) the hammer mechanism 312, the exemplary graft harvesting device 300 can include a crank-actuated mechanism (not shown), e.g., a crank-actuated screw mechanism, and the like, for lowering the broach 322 and/or the cutting member 324 into the cartilage 304 and/or the defect region cavity 302. The crank-actuated mechanism generally reduces the potential toggle effect of the hammer mechanism 312 when driving the broach 322 and/or the cutting member 324 into the cartilage 304. Thus, in some embodiments, the crank-actuated mechanism can be implemented to initially insert and fixate the cutting member 324 and/or the broach 322 into the cartilage 304 until a steady position has been established. The hammer mechanism 312 can then be implemented to drive the cutting member 324 and/or the broach 322 to the full desired depth. In some embodiments, the crank-actuated mechanism can be implemented to drive the cutting member 324 and/or the broach 322 the full desired depth into the cartilage 304. The exemplary crank-actuated mechanism generally ensures the proper orientation, e.g., positioning, angle, and the like, of the graft harvesting device 300 relative to the cartilage 304 and/or the captured surface topography surrounding the defect region cavity 302.

The broach flange 314 can have a scalloped surface, and generally mechanically interlocks with the broach 322 to permit the broach 322 to be axially translated a maximum distance equal to the length of the broach flange path 318. The broach flange 314 can also be rotated in a direction indicated by broach flange arrows 326 to lock and/or unlock the axial movement of the broach 322. The functionality of the broach flange 314 will be discussed in greater detail below with respect to the disposable portion B.

The disposable portion B of the exemplary graft harvesting device 300 generally includes a connecting shaft 328, a lower flange 330, a locking mechanism 332, a cutting member 324, a broach 322 and a plurality of elongated rod members 334. The configuration and/or dimensions of the components of the disposable portion B can be customized to meet the needs of a user based upon, e.g., the size and/or geometry of the defect region cavity 302 of the cartilage 304 and/or the template 200 utilized. The connecting shaft 328 can be configured and dimensioned to interlock the reusable portion A components with the disposable portion B components in a mechanically functioning manner. Thus, the modular and/or disposable design of the disposable portion B permits the disposable portion B components to engage the reusable portion A, which can further be implemented for axially advancing and/or retracting the cutting member 324 and the broach 322 and for ejecting a graft plug post-harvesting. In particular, the connection between the reusable portion A and the disposable portion B permits the broach 322 to mechanically interlock and/or interact with the broach flange 314 and further permits the cutting member 324 to rigidly interlock and/or interact with the handle 308. The modularity of the disclosed graft harvesting device 300 can reduce the costs associated with the replacement of instruments required for the procedures discussed herein relative to the procedures taught by the prior art.

Still with reference to the disposable portion B of FIG. 46, the cutting member 324 can be rigidly secured to the connecting shaft 328. The cutting member 324 can also be defined by a hollow body and a serrated edge geometrically configured to match, e.g., the preformed geometric shape opening 220′″ of the template 200′″ of FIGS. 43-45, and can be used for harvesting a graft plug from a harvest location. In some embodiments, the connecting shaft 328 can be defined by two concentric shafts, e.g., an inner and outer connecting shaft. The outer connecting shaft can rigidly secure the cutting member 324 to the elongated shaft 306 and/or the handle 308. The inner connecting shaft can be positioned inside the outer connecting shaft and can connect the broach 322 to the broach flange 314. Thus, the inner connecting shaft can be axially translated independently of and relative to the outer connecting shaft. Further, the broach 322 can be positioned within the hollow body of the cutting member 324 and can be axially translated relative to the cutting member 324. The broach 322 can also interlock with the broach flange 314 for securely locking the broach 322 in a desired position. In particular, the broach 322 can be defined by, e.g., a rough surface, a plurality of downwardly and/or upwardly facing serrated edges/ridges, and the like, and can function as a reamer, thus permitting the user to “clean”, e.g., file away, provide finishing, and the like, the potential rough inner surfaces of the defect region cavity 302 created by a cutter to ensure a smooth fitting of the donor graft plug. The hammer mechanism 312 can be implemented to axially drive the broach 322 into the defect region cavity 302 by axially hammering against the top surface 316 of the handle 308 to drive the graft harvesting device 300 in the direction of the cartilage 304.

A plurality of elongated rod members 334 can be secured to the disposable portion B around the outer perimeter of the cutting member 324 and can function substantially similarly to the elongated rod members of the trial members discussed above. The elongated rod members 334 act as male components and can be inserted into the complementary female components, e.g., apertures, located on the locking mechanism 332. Thus, as the graft harvesting device 300 is lowered against the cartilage 304 surface and the broach 322 is inserted into the defect region cavity 304, the elongated rod members 334 can be free to axially translate through the female components, e.g., apertures, of the locking mechanism 332 to capture the peripheral surface topography of the defect region cavity 302. In general, the locking mechanism 332 acts substantially similarly to the locking mechanisms described above for securing the elongated rod members 334 in a position representative of the peripheral surface topography of the defect region cavity 302 in order to locate a topographically matching harvest location.

The groove/track 336 between the top and bottom portions of the locking mechanism 332 can further receive, e.g., a rubber band and/or an O-ring element, for further frictionally locking the elongated rod members 313 in position. In some embodiments, rather than or in combination with the rubber band and/or O-ring element, the locking mechanism 332 can be fabricated from a material, e.g., a rubber, which imparts a frictional force against the plurality of elongated rod members 334 to lock the elongated rod members 334 within the locking mechanism 332 when a translational force is not being applied to the elongated rod members 334. Thus, when an axial force is applied to the distal end of the elongated rod members 334, the elongated rod members 334 can translate axially through the complementary female components of the locking mechanism 332. However, when no axial force is applied to the elongated rod members 334, the locking mechanism 332 can secure the elongated rod members 334 in the most recent position. In some embodiments, the graft harvesting device 300 can include a drive mechanism (not shown) for electronically controlling the translation of the elongated rod members 334 relative to the locking mechanism 332 and/or the cartilage 304. For example, the drive mechanism can be implemented to translate the elongated rod members 334 against the surface of the cartilage 304 around the defect region cavity 302 to capture the peripheral surface topography and the locking mechanism 332 can be implemented to lock-in the position of the elongated rod members 334 for further use of the captured surface topography. In addition, each of the plurality of elongated rod members 334 can include an elongated rod member cap 338 to prevent the elongated rod members 334 from axially passing through and out of the locking mechanism 332.

The lower flange 330 of the disposable portion B can be secured around and be axially translatable along the connecting shaft 328. In some embodiments, the lower flange 330 can act as a “stop”, e.g., an even surface which provides a limit to the axial translation of the plurality of elongated rod members 334 in an upward direction. In some embodiments, the lower flange 330 can be manually and/or electronically translated down along the connecting shaft 328 to “reset”, e.g., reposition, the plurality of elongated rod members 334. For example, by translating the lower flange 330 in a downward direction along the connecting shaft 328, a downward axial force can be applied to the elongated rod member caps 338 to reposition the plurality of elongated rod members 334 in a desired position, e.g., a position of maximum extension below the locking mechanism 332. The position of maximum extension can also be defined by the elongated rod member caps 338 positioned substantially adjacent to a top surface of the locking mechanism 332.

As described above, in some exemplary embodiments, the plurality of elongated rod members 334 can be electronically actuated to move toward and against the cartilage 304 surface. For example, the locking mechanism 332 can be electronically and/or manually translatable axially relative to the connecting shaft 328. The locking mechanism 332 can thereby be retracted, i.e., translated to be positioned substantially adjacent to the lower flange 330. Axially translating the locking mechanism 332 against the lower flange 330 generally acts to “reset”, e.g., reposition, the plurality of elongated rod members 334 such that the plurality of elongated rod members 334 can be fully extended in the direction of the cartilage 304 with the elongated rod member caps 338 positioned adjacent to the locking mechanism 332.

In some embodiments, the graft harvesting device 300 can be lowered against the cartilage 304 surface and the broach 322 can be inserted into the defect region cavity 302 without affecting the position of the plurality of elongated rod members 334. When the broach 322 has been positioned within the defect region cavity 302, the locking mechanism 332 can be electronically actuated by, e.g., a switch (not shown), to axially translate in the direction of the cartilage 304 surface. It should be understood that as the locking mechanism 332 axially translates in the direction of the cartilage 304 surface, the plurality of elongated rod members 334 also axially translate with the locking mechanism 332. Thus, when the extended distal ends of the elongated rod members 334 reach the cartilage 304 surface, the axial force applied against the distal ends of the elongated rod members 334 by the continued translation of the locking mechanism 332 can force the elongated rod members 334 to translate axially through the complementary female components of the locking mechanism 332 to capture the peripheral surface topography of the defect region cavity 302. When the peripheral surface topography has been fully captured, the locking mechanism 332 can be electronically actuated to stop the axial translation along the connecting shaft 328 and the elongated rod members 334 can maintain the captured surface topography of the area surrounding the defect region cavity 302. If the repositioning or “reset” of the plurality of elongated rod members 334 is desired, the locking mechanism 332 can be electronically and/or manually actuated to translate against the lower flange 330 to position the elongated rod members 334 in a fully extended position. Optionally, the lower flange 330 can be axially translated (electronically and/or manually) against the locking mechanism 332 to “reset” the position of the plurality of elongated rod members 334.

As described above with respect to the exemplary trial members, the elongated rod members 334 can also include a color variation (not shown) to alert a user when the elongated rod members 334 may be insufficient for measuring the topography of the area surrounding the defect region cavity 302. For example, each elongated rod member 334 can include a portion of differently colored paint, colored markings and/or varying surface texture indicating to the user that the elongated rod members 334 have been translated almost the full length through the locking mechanism. In some embodiments, rather than or in combination with the visual indicators, the graft harvesting device 300 can include an auditory indicator which emits at least one signal indicating the position of the elongated rod members 334 and/or the adequacy of the elongated rod members 334 for measuring the cartilage 304 topography. Thus, as the elongated rod members 334 are lowered and pressed against the cartilage 304 surface surrounding the defect region cavity 302, if the length of the elongated rod members 334 is insufficient to capture the topographical variation, the colored markings and/or the auditory indicator can notify the user that, e.g., longer elongated rod members 334 should be utilized. The disposable portion B can then be switched to one having longer elongated rod members 334.

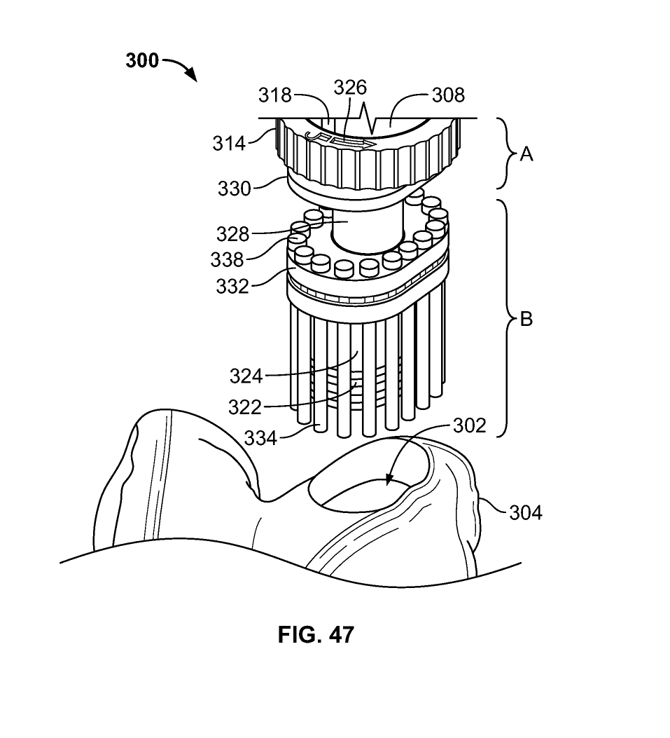

Turning now to FIG. 47, the exemplary graft harvesting device 300 is depicted in preparation for insertion of the broach 322 into the defect region cavity 302. In particular, the broach flange 322 has been translated along the broach flange path 318 to the lowest portion of the broach flange path 318, thereby axially translating the broach 322 from a position inside the body of the cutting member 324 to a position protruding a predetermined distance out of the body of the cutting member 324. The broach flange 322 can also be rotated in the appropriate direction shown by to broach flange arrows 326 to lock the broach 322 in the protruding position. The predetermined distance which the broach 322 protrudes out of the cutting member 324 generally corresponds to the depth of the donor graft plug required to fill the defect region cavity 302. The plurality of elongated rod members 334 can be axially translated to a position of maximum extension below the locking mechanism 332, e.g., the elongated rod member caps 338 abut the top surface of the locking mechanism 332 and the elongated rod members 334 can be extended below a bottom surface of the broach 322. Thus, as the broach 322 is lowered into the defect region cavity 302, the elongated rod members 334 can contact the cartilage 304 surface periphery surrounding the defect region cavity 302 prior to the broach 322 contacting the defect region cavity 302. This ensures that an accurate peripheral surface topography can be obtained by the elongated rod members 334 simultaneously to the “cleaning” of the surfaces of the defect region cavity 302.

As described above, in some exemplary embodiments, the broach 322 can be inserted into the defect region cavity 302 independently of the surface topography capture step and the locking mechanism 332 can then be electronically or manually actuated to lower the plurality of elongated rod members 334 against the cartilage 304 surface to capture the periphery surrounding the defect region cavity 302. The broach 322 can be axially driven into the defect region cavity 302 by, e.g., hammering the hammer mechanism 312 against the top surface 316 of the handle 308, actuating the crank-actuated mechanism to lower the broach 322 into the defect region cavity 302, combinations thereof, and the like.

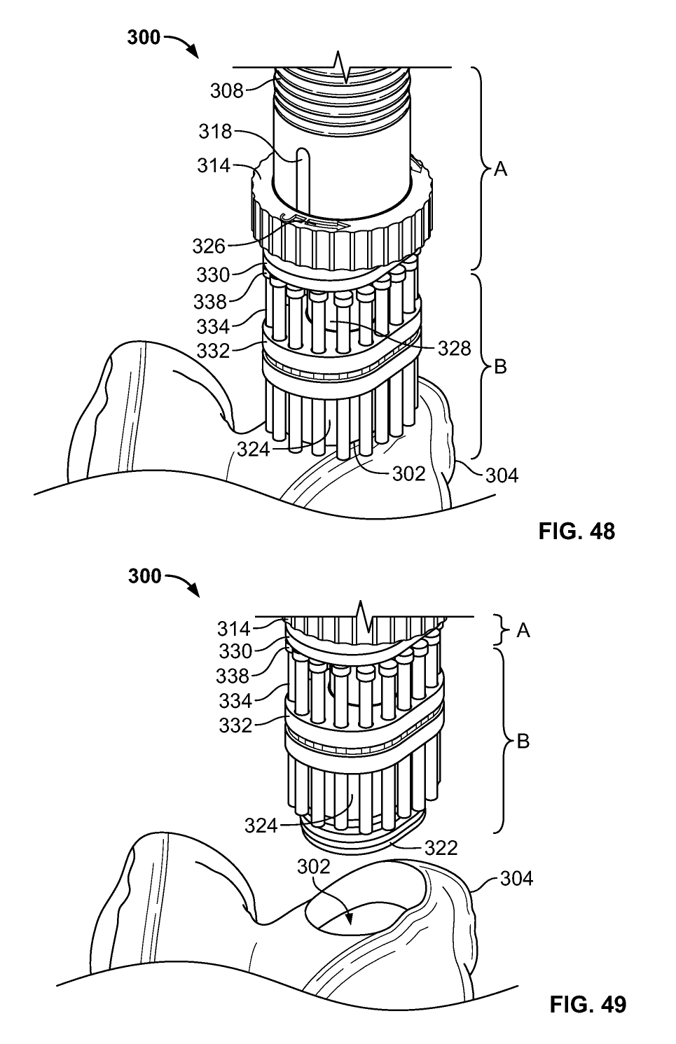

With reference to FIG. 48, the broach 322 of the exemplary graft harvesting device 300 has been inserted into the defect region cavity 302 and the plurality of elongated rod members 334 have simultaneously captured the peripheral surface topography of the defect region cavity 302. In particular, as the graft harvesting device 300 are lowered and/or hammered into the defect region cavity 302, except for the elongated rod members 334, all of the components of the disposable portion B remain axially fixed relative to each other. However, it should be understood that the peripheral surface topography of the defect region cavity 302 can also be captured by electronically translating the locking mechanism 332 along the connecting shaft 328 to force the plurality of elongated rod members 334 against the cartilage 304 surface independently from the insertion of the broach 322.

Turning to FIG. 49, the broach 322 of the exemplary graft harvesting device 300 has been removed from the defect region cavity 302. It should be understood that insertion and/or removal of the broach 322 from the defect region cavity 302 acts to substantially “clean” the inner surfaces of the defect region cavity 302 from undesired cartilage remaining after creation of the defect region cavity 302 with the cutters described above. In general, the removal of the broach 322 from the defect region cavity 302 can be performed by, e.g., pulling on the handle 308 in an axially upward direction away from the defect region cavity 302, utilizing the hammer mechanism 312 to hammer against the bottom surface 320 of the top cap 310, and the like. Further, as the broach 322 is removed from the defect region cavity 302, all of the components of the disposable portion B can remain axially fixed relative to each other. Thus, as can be seen in FIG. 49, after removal of the broach 322 from the defect region cavity 302, the plurality of elongated rod members 334 can remain fixed by the locking mechanism 332 in a position representative of the peripheral surface topography of the defect region cavity 302.

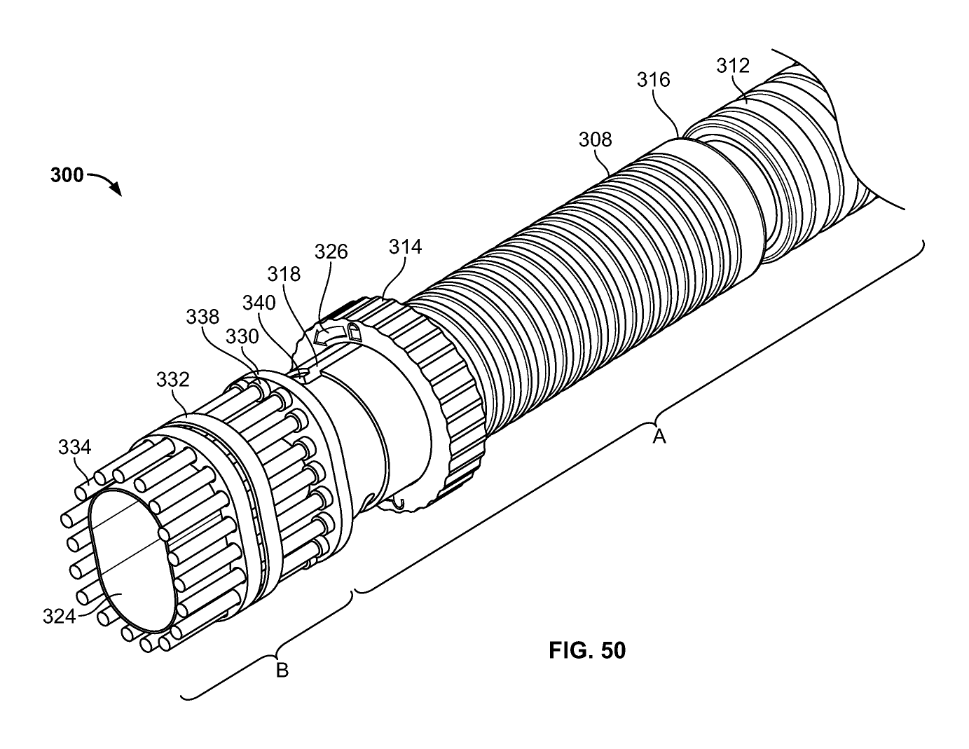

With reference to FIG. 50, once the defect region cavity 302 has been “cleaned” with the broach 322 and the peripheral surface topography of the defect region cavity 302 has been obtained or captured by the elongated rod members 334, the broach 322 can be retracted axially in an upward direction by unlocking and translating the broach flange 314 to the highest position along the broach flange path 318. In particular, the broach flange 314 can be spring-loaded to automatically axially translate the broach flange 314 to the highest position along the broach flange path 318 when the broach 322 and the broach flange 314 have been unlocked from a protruding position. However, it should be understood that the exemplary broach flange 314 can also be manually translated to the highest position along the broach flange path 318 and the broach 322 can be locked in a retracted position by, e.g., rotating the broach flange 314 along a broach flange locking path (not shown) similar to the broach flange locking path 340 located at the lowest position along the broach flange path 318 for locking the broach 322 in a protruding position. The broach flange locking path 340 can be defined by a path oriented at approximately a 90° angle relative to the broach flange path 318 along which the broach flange 314 (and a protrusion (not shown) within the broach flange 314) can rotate, thus preventing the broach flange 314 from entering and being translatable along the broach flange path 318.

The retraction of the broach 322 enables the user to implement the peripheral surface topography of the defect region cavity 302 captured by the plurality of elongated rod members 334 to identify and/or locate, e.g., match, a harvest location having a complementary surface topography. For example, a user can position and translate the elongated rod members 334 with the captured peripheral surface topography along available harvest location surfaces to determine which harvest location surface topography substantially matches and/or aligns with the captured surface topography of the elongated rod members 334. Once a complementary surface topography of a harvest location has been located, the user can utilize the hammer mechanism 312 to axially drive the cutting member 324 downward into an allograft and/or autograft donor location for harvesting a donor graft plug. It should be understood that the donor location may be, e.g., autograft, allograft, xenograft, synthetic, and the like.

The length of the cutting member 324 can be a predetermined and customized length based on the depth of the defect region cavity 302. In addition, the retracted distance of the broach 322 into the inner cavity within the cutting member 324 ensures that the depth of the inner cavity of the cutting member 324 can be complementary to the depth of the defect region cavity 302. Thus, as the user axially drives the cutting member 324 into the allograft and/or autograft donor location, when the bottom surface of the broach 322 contacts the top surface of the allograft and/or autograft donor location, the cutting member 324 can be prevented from moving further into the allograft and/or autograft donor location and the user can understand that the desired predetermined height of the harvest graft plug has been reached. Once the desired harvest graft plug height has been reached, the cutting member 324 can be removed from the allograft and/or autograft donor location with the untrimmed harvest graft plug located inside the cavity of the cutting member 324 by utilizing the hammer mechanism 312 to axially hammer against the bottom surface 320 of the top cap 310.

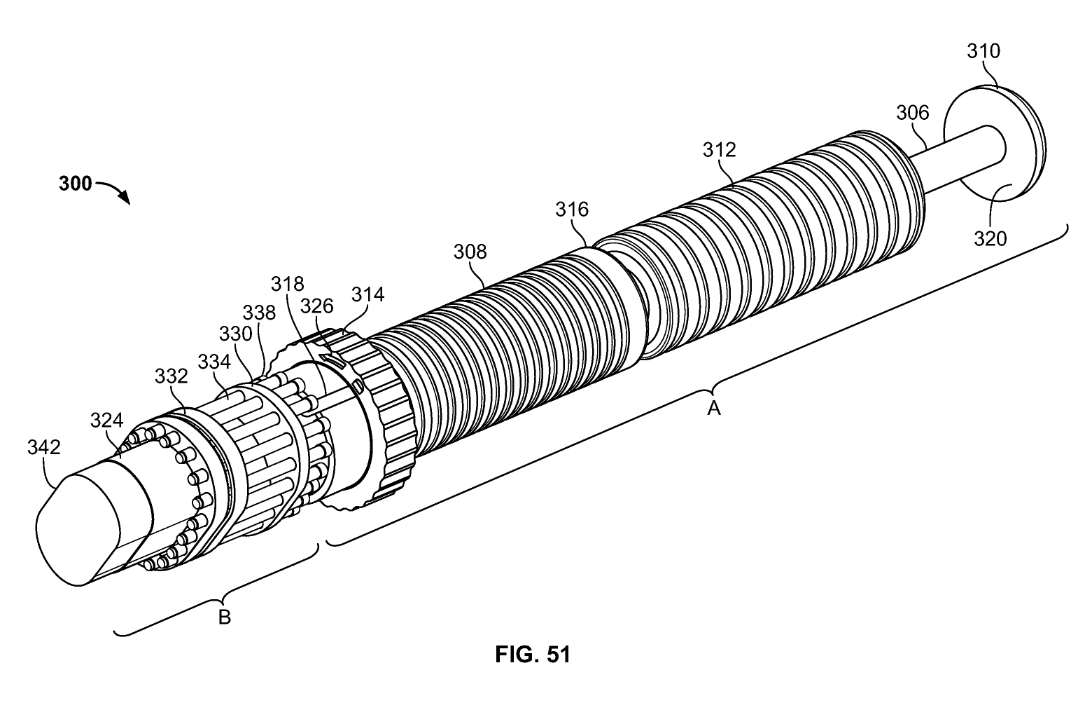

Turning now to FIG. 51, the exemplary graft harvesting device 300 is illustrated after removal from the allograft and/or autograft donor location, including a bottom portion of the untrimmed donor graft plug 342 protruding out of the cutting member 324. As discussed above, the inner cavity of the cutting member 324 defines the geometrical shape and height required for the donor graft plug 342 to fill the defect region cavity 302. Thus, the portion of the donor graft plug 342 protruding past the lowest point of the cutting member 324 defines additional and/or unwanted graft material which can result during removal of the cutting member 324 from the donor location. In particular, the additional and/or unwanted graft material can be removed prior to inserting the donor graft plug 342 into the defect region cavity 302, while the top surface of the donor graft plug 342 located in the cavity of the cutting member 324 can be defined by the desired surface topography matching the surface topography surrounding the defect region cavity 302.

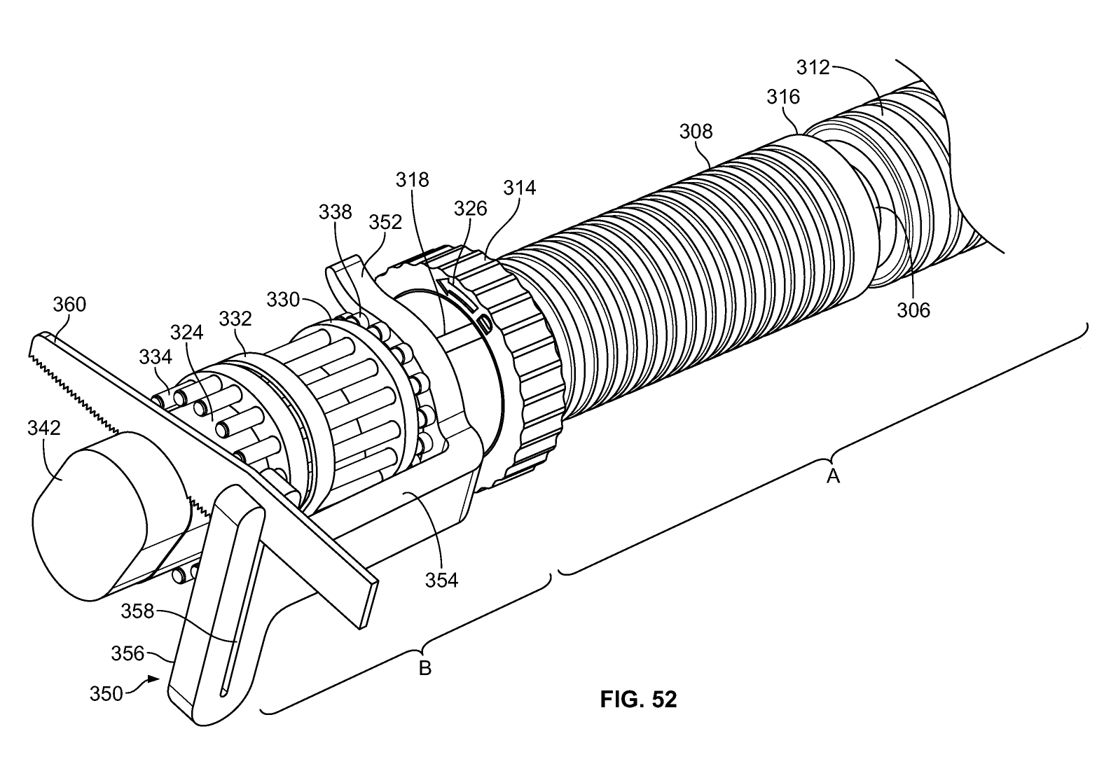

With reference to FIG. 52, in order to remove, e.g., trim, the additional and/or unwanted graft material from the donor graft plug 342 and create a desired donor graft plug 342 depth dimension, an exemplary cutter guide 350 can be implemented. The exemplary cutter guide 350 generally includes an attachment member 352, a connecting member 354, a cutter guide head 356 and a cutter guide channel 358. The attachment member 352 can be configured and dimensioned as, e.g., a flexible C-shaped clip with a spring-like property, thus permitting a user to fit and secure the attachment member 352 relative to and/or around the elongated shaft 306 and/or the handle 308. The spring-like property of the attachment member 352 can be sufficiently strong to prevent unwanted motion of the cutter guide 350 during trimming of the donor graft plug 342. Although illustrated as a detachable member, it should be understood that the cutter guide 350 can also be configured as an integral component of the reusable portion A.

The connecting member 354, e.g., the arm, can connect the attachment member 352 and the cutter guide head 356 and can be configured and dimensioned to extend the cutter guide head 356 over the components of the disposable portion B. The connecting member 354 can also be configured and dimensioned to align the cutter guide channel 358 with the distal end of the cutting member 324. It should be understood that the connecting member 354 can be configured as a telescoping connecting member 354, thus permitting a user to vary the length of the connecting member 354 as needed depending on the configurations and/or dimensions of the disposable portion B components.

The cutter guide head 356 includes a cutter guide channel 358 for passing through and aligning a trimming instrument 360, e.g., a saw, with the distal end of the cutting member 324, thereby permitting an accurate trimming of the unwanted graft material of the donor graft plug 342 and ensuring a desired donor graft plug 342 depth of, e.g., about 10 mm, depending on the defect region 302 and/or joint being repaired. It should be understood that alternative desired depths can be obtained by implementing appropriately customized components of the disposable portion B, e.g., a depth of about 6 mm for a shoulder joint. Due to the desired donor graft plug 342 depth being located inside the cavity of the cutting member 324, other than aligning the trimming instrument 360 with the distal end of the cutting member 324, the user is generally not required to axially size the depth of the donor graft plug 342. Further, the implementation of the cutter guide 350 in conjunction with the desired donor graft plug 342 depth being located inside the cavity of the cutting member 324 eliminates the need for utilizing, e.g., a chisel, which could potentially dislodge the donor graft plug 342 from the graft harvesting device 300 and result in an inaccurate donor graft plug 342 geometry.

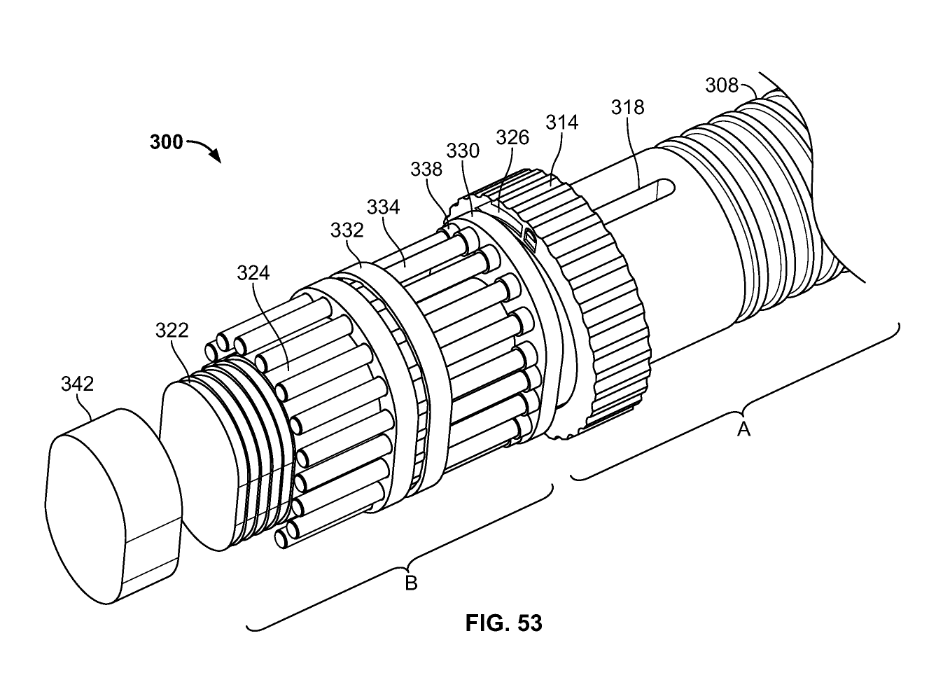

With reference to FIG. 53, the ejection of the donor graft plug 342 from the cutting member 324 of the exemplary graft harvesting device 300 is illustrated. In particular, while the donor graft plug 342 is located in the inner cavity of the cutting member 324, the broach flange 314 can be unlocked and translated down the broach flange path 318, thereby axially translating the broach 322 through and out of the inner cavity of the cutting member 324. The pressure from the translating broach 322 acts to eject the donor graft plug 342 out of the cutting member 324. It should be understood that the broach 322 can be implemented to eject the donor graft plug 342 out of the cutting member 324 for independent insertion into the defect region cavity 302 and/or the donor graft plug 342 can be ejected out of the cutting member 324 directly into the recipient site, e.g., the defect region cavity 302. Implementing the large surface area of the bottom of the translating broach 322, rather than an additional instrument, e.g., a small rod with a single point of force application, provides a substantially even force distribution on the top surface of the donor graft plug 342, thus preventing damage to the donor graft plug 342 during ejection from the graft harvesting device 300. In particular, the improved force distribution from the broach 322 onto the top surface of the donor graft plug 342 prevents damage to the desired surface topography of the donor graft plug 342 which is complementary to the surface topography surrounding the defect region cavity 302 during ejection.

As described above, in exemplary embodiments where the defect region cavity 302 is configured as non-symmetrical, e.g., slightly tapered, the cutting member 324 can be configured and dimensioned to substantially match the non-symmetrical defect region cavity 302 to harvest a complementary donor graft plug 342. The non-symmetrical configuration of the defect region cavity 302 and the harvested donor graft plug 342 can ensure that the proper orientation of the donor graft plug 342 relative to the defect region cavity 302 can be maintained, e.g., the depth, surface topography, and the like, are property maintained by allowing insertion of the donor graft plug 342 into the defect region cavity 302 only when the non-symmetrical configurations have been aligned.

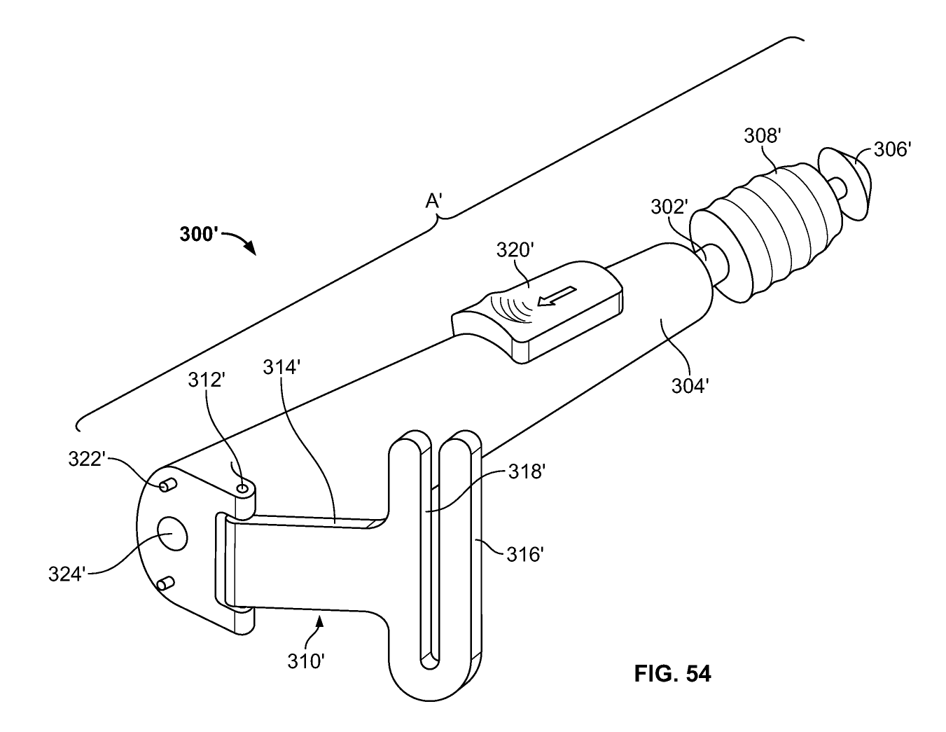

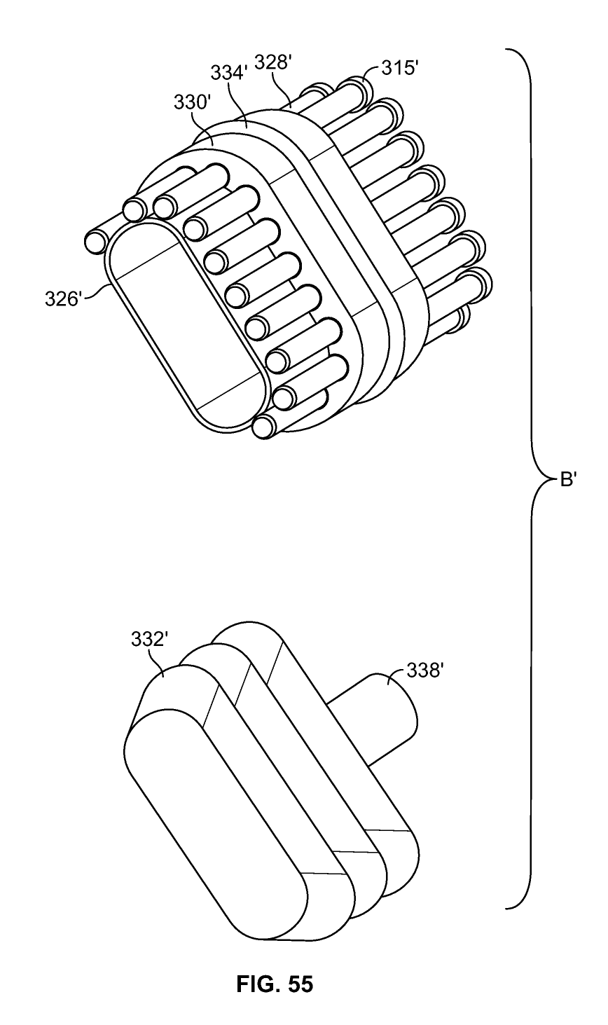

Turning now to FIG. 54, an alternative exemplary embodiment of a reusable portion A′ of a graft harvesting device 300′ is presented. In particular, the exemplary graft harvesting device 300′ generally includes the reusable portion A′ of FIG. 54 and the disposable portions B′ of FIG. 55. The reusable portion A′ of FIG. 54 can be configured and functions substantially similarly to the reusable portion A discussed previously, generally including an elongated shaft 302′, a handle 304′, a top cap 306′ and a hammer mechanism 308′. The reusable portion A′ can further include an integral cutter guide 310′ configured as an attachment member 312′, e.g., a hinge, a connecting member 314′, a cutter guide head 316′ and cutter guide channel 318′. It should be understood that the connecting member 34′ can be configured as a telescoping connecting member 314′, thus permitting a user to vary the length of the connecting member 314′ as needed depending on the configurations and/or dimensions of the disposable portion B′ components.

The graft harvesting device 300′ can include a spring-loaded button 320′ which can function substantially similarly to the broach flange 314 of FIG. 46. In particular, the spring-loaded button 320′ can be actuated to, e.g., retract and/or protrude the broach 332′ of FIG. 55, eject a donor graft plug, reset the plurality of elongated rod members 328′ of FIG. 55 with a lower flange (not shown) similar to the lower flange 330 discussed above, and the like. The reusable portion A′ components can be detachably secured, e.g., mechanically interlocked, to the disposable portion B′ components through a mechanical connection, including pins 322′ and a shaft aperture 324′.

With reference to FIG. 55, an alternative exemplary embodiment of a disposable portion B′ is presented. In particular, the assembly of the cutting member 326′, the plurality of elongated rod members 328′, the locking mechanism 330′ and the broach 332′ generally represent the disposable portion B′ according to the present disclosure. The cutting member 326′, the plurality of elongated rod members 328′ and the locking mechanism 330′ of FIG. 55 can be substantially similar to those discussed previously. It should be noted that FIG. 55 further illustrates the addition of a locking element 334′, e.g., a rubber band and/or an O-ring, fitted into the groove between the top and bottom surfaces or portions of the locking mechanism 330′. As previously mentioned, the locking element 334′ can frictionally prevent the axial motion of the plurality of elongated rod members 328′ relative to the locking mechanism 330′. The elongated rod members 328′ can include an elongated rod member cap 336′ for preventing full translation of the elongated rod members 328′ through and out of the locking mechanism 330′. The broach 332′ of FIG. 55 can be substantially similar to the broach 322 discussed previously, further including a broach shaft 338′ for mating, e.g., mechanically interlocking, with the shaft aperture 324′ of the reusable portion A′. Thus, similar to the graft harvesting device 300 described above, the exemplary graft harvesting device 300′ of FIGS. 54 and 55 can be implemented to, e.g., clean a defect region cavity, harvest a graft donor plug, and the like.

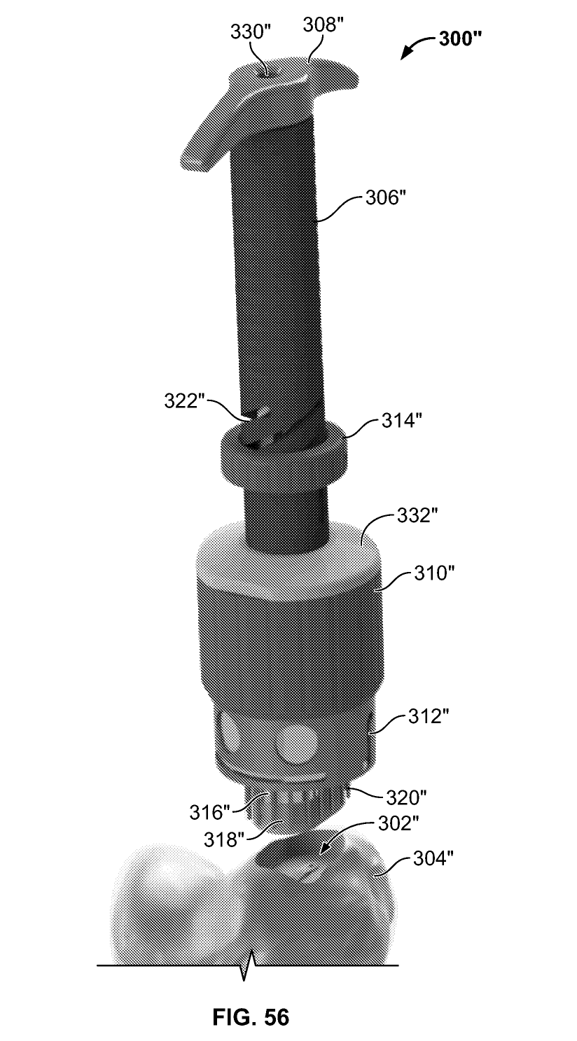

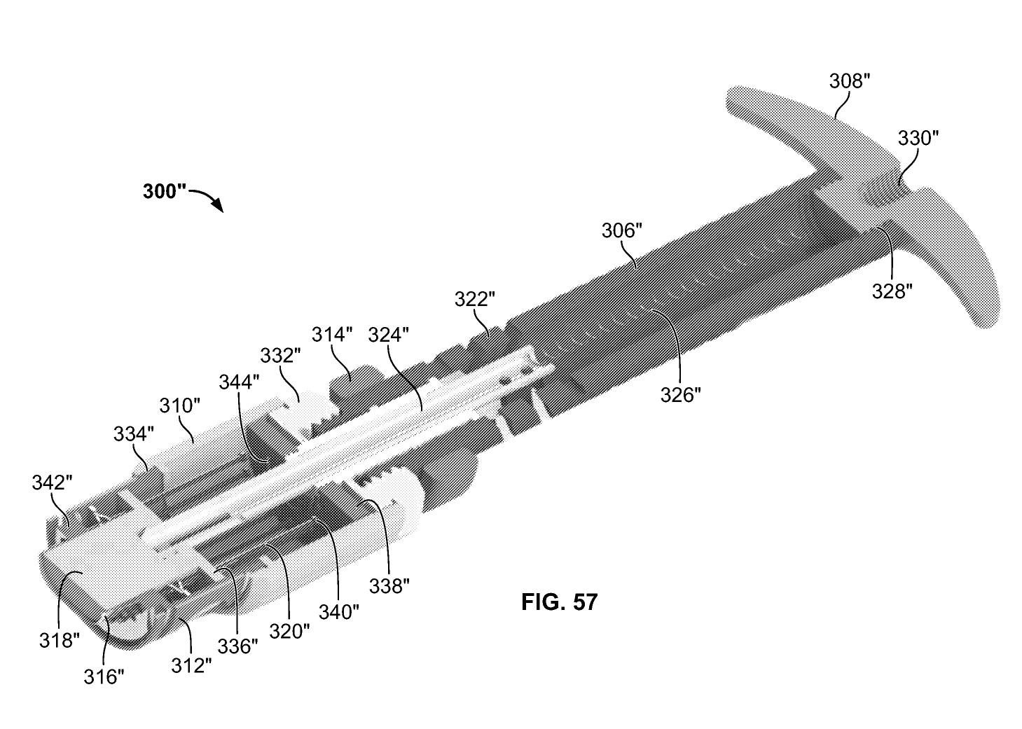

Turning now to FIGS. 56-63, an exemplary graft harvesting device 300″ is provided for harvesting an implant for a defect region cavity 302″, i.e., an implant region, in cartilage 304″ of a patient in accordance with the present disclosure and advantageous systems/methods as described, for example, in PCT applications WO 2009/154691 A9 (corrected version) and WO 2011/008968 A1, which have been previously incorporated herein by reference. With reference to the perspective and cross-sectional views of the graft harvesting device 300″ in FIGS. 56 and 57, the exemplary graft harvesting device 300″ generally includes a handle 306″, an actuator handle 308″ (e.g., a crank-actuated handle), a mechanical housing 310″, and a trimmer guide 312″ (e.g., a cutter guide). The handle 306″ can be fabricated from a material which ensures a secure grip by a user around the handle 306″, e.g., a rubber or foam material, and can include ridges thereon for improving the grip capability. In some embodiments, the handle 306″ can be fabricated from a rigid material, e.g., a stainless steel, and can be covered with a comfortable material suitable for gripping by a user.

The graft harvesting device 300″ generally includes a broach flange 314″, a cutting member 316″ (e.g., a punch), a broach 318″, and a plurality of elongated rod members 320″ extending around the perimeter of the cutting member 316″, e.g., a trial device section of the graft harvesting device 300″. The handle 306″ generally includes a helical broach flange path 322″ along which the broach 318″ can travel as the broach 318″ is axially rotated by a user. The cutting member 316″ defines a blade, e.g., a serrated blade, a non-serrated blade, and the like, at a distal end, and defines a cavity within a perimeter of the cutting member 316″. The blade of the cutting member 316″ can be configured and dimensioned to be driven into a donor location for harvesting a bone and cartilage graft for implanting into the defect region cavity 302″. Although illustrated as substantially oval in shape, it should be understood that the cutting member 316″ can be configured in a variety of shapes depending on the configuration and dimensions of the defect region or the defect region cavity 302″. The cavity of the cutting member 316″ can be configured and dimensioned to receive therein the broach 318″. The broach 318″ can include a scalloped surface, e.g., ridges, blades, and the like, such that when the broach 318″ is introduced into the defect region cavity 302″, the inner surfaces of the defect region cavity 302″ can be substantially cleaned or smoothed by removing any undesired cartilage remaining after creation of the defect region cavity 302″ with the cutters discussed above. In some embodiments, the broach 318″ can be introduced into the implant region 302″ to confirm, e.g., the depth, geometry, orientation, and the like, of the implant to be harvested.

With specific reference to FIG. 57, the graft harvesting device 300″ can include an elongated shaft 324″ axially located therein and fixedly secured to the handle 306″. The elongated shaft 324″ can be fixedly secured to the broach 318″ and/or the cutting member 316″ for axially driving the broach 318″ and/or the cutting member 316″ into and out of the cartilage 304″. In some embodiments, the elongated shaft 324″ can be mechanically connected to the actuator handle 308″ with a spring 326″. The spring 326″ can create a spring-loaded effect between the elongated shaft 324″ and the actuator handle 308″. Thus, when the actuator handle 308″ is rotated by a user along complementary threads 328″ on the actuator handle 308″ and the elongated shaft 324″ in one direction, e.g., clockwise, the actuator handle 308″ can be lowered deeper into the handle 306″ and the spring 326″ can be compressed to generate a higher force against the elongated shaft 324″. Similarly, if the actuator handle 308″ is rotated in an opposite direction, e.g., counter-clockwise, the actuator handle 308″ can be raised higher out of the handle 306″, thereby expanding the spring 326″ and reducing the force generated against the elongated shaft 324″.

The force generated by mechanical interaction between the actuator handle 308″ and the elongated shaft 324″ can be utilized to drive the cutting member 316″ and/or the broach 318″ into and out of the cartilage 304″. For example, compressing the spring 326″ and generating an axial force against the elongated shaft 324″ can drive the broach 318″ into the defect region cavity 302″. In some embodiments, expanding the spring 326″ and reducing the force generated against the elongated shaft 324″ can generate an axial pulling force which retrieves the broach 318″ and/or the cutting member 316″ from the cartilage 304″. In some embodiments, rather than implementing the actuator handle 308″, the cutting member 316″ and/or the broach 318″ can be driven into the cartilage 304″ by, e.g., manually pushing the graft harvesting device 300″ into the cartilage 304″, hammering against the actuator handle 308″, connecting an ancillary device, such as a slap hammer or a crank-actuated mechanism, to the threaded aperture 330″ on the top surface of the actuator handle 308″, and the like.

The mechanical housing 310″ can be fixedly secured to the handle 306″ by a fixation element 332″ and the trimmer guide 312″ can be fixedly secured to the mechanical housing 310″ by complementary threads 334″. The mechanical housing 310″ generally includes therein the elongated rod members 320″, a locking mechanism 336″ and a lower flange 338″. The locking mechanism 336″ generally includes a plurality of radially spaced apertures passing therethrough configured and dimensioned to receive the elongated rod members 320″. The elongated rod members 320″ can translate through the apertures independently from each other. Similar to the locking mechanism 332 discussed above, the locking mechanism 336″ can be fabricated from, e.g., a rubber, to impart a frictional force against the elongated rod members 320″ to capture the position of the elongated rod members 320″ after the surface topography surrounding the defect region cavity 302″ has been captured. In some embodiments, an O-ring or a rubber band can be implemented to impart the frictional force on the elongated rod members 320″. Each of the elongated rod members 320″ generally includes an elongated rod member cap 340″ to prevent the elongated rod members 320″ from passing fully through the apertures in the locking mechanism 336″. In some embodiments, the locking mechanism 336 can be fabricated from a rigid material and can be secured to the elongated shaft 324″ to impart the force generated by the actuated handle 308″ against the cutting member 316″ and/or the broach 318″. In some embodiments, the feature indicated in FIG. 57 as the locking mechanism 336″ can be utilized only as a force-imparting feature and an elongated rod member fixation plate 342″ can be implemented as a locking mechanism for the elongated rod members 320″.

The lower flange 338″ can be mechanically connected to the broach flange 314″ by an internal spring 344″. In some embodiments, axially rotating the broach flange 314″ along the broach flange path 322″ to a position adjacent to the fixation element 332″ can translate the lower flange 338″ down and against the elongated rod members 320″ by compressing the spring 344″ and generating a force on the lower flange 338″ to fully extend the elongated rod members 320″ from a distal end of the graft harvesting device 300″. The position of the elongated rod members 320″ can thereby be “reset” into a fully extended position. Axially rotating the broach flange 314″ along the broach flange path 322″ away from the fixation element 332″ in the direction of the actuator handle 308″ can reduce the force generated by the spring 344″ and can lift the lower flange 338″ to a position substantially adjacent to the fixation element 332″. In some embodiments, the broach flange 314″ can include broach flange indicators or arrows (not shown) thereon to indicate the direction in which the broach flange 314″ should be rotated to raise or lower the broach flange 314″ relative to the handle 306″.

In some embodiments, the broach flange 314″ can be implemented for retracting and withdrawing the broach 318″ from the cavity within the cutting member 316″. For example, rotating the broach flange 314″ down against the fixation element 332″ can withdraw the broach 318″ from the cutting member 316″, while rotating the broach flange 314″ up and away from the fixation element 332″ can retract the broach 318″ deep into the cavity of the cutting member 316″ to allow use of the cutting member 316″.

Similar to the graft harvesting device 300 discussed above, once the elongated rod members 320″ have been fully extended by the lower flange 338″, the broach 318″ can be introduced into the defect region cavity 302″ and the elongated rod members 320″ can capture the surface topography surrounding the defect region cavity 302″. In some embodiments, the broach 318″ can be introduced into the defect region cavity 302″ and the elongated rod members 320″ can be manually or electronically actuated to translate against the cartilage 304″ surface independently from the movement of the broach 318″. The captured surface topography can be further implemented to locate a substantially complementary surface topography at a harvest site for harvesting a bone and cartilage graft for implanting into the defect region cavity 302″.

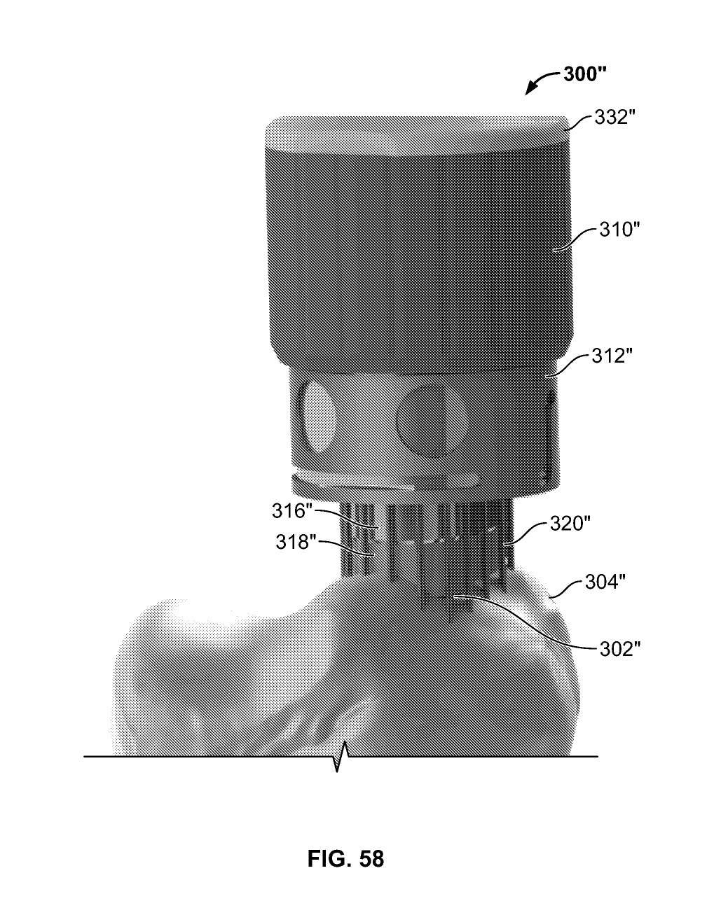

FIG. 58 illustrates the broach 318″ inserted into the defect region cavity 302″. In particular, the broach 318″ has been extended out of the cutting member 316″ and driven into the defect region cavity 302″. The elongated rod members 320″ shown in FIG. 58 have been positioned against the surface of the cartilage 304″ surrounding the defect region cavity 302″ and have translated independently of each other to capture the peripheral surface topography.

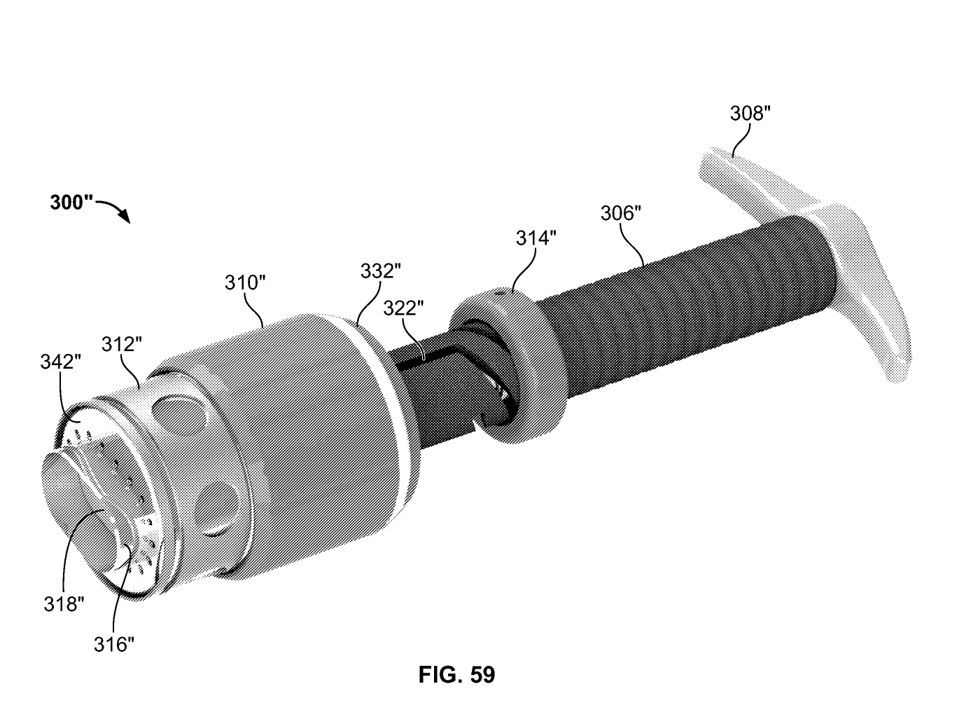

Once the desired information, i.e., the peripheral surface topography, has been obtained by the elongated rod members 320″ and the broach 318″ has been used to clean the defect region cavity 302″, the broach 318″ can be withdrawn from the defect region cavity 302″. As shown in FIG. 59, the broach 318″ can further be axially drawn into the body of the graft harvesting device 300″, i.e., the cavity within the cutting member 316″, thereby exposing the cutting member 316″ for use. The captured peripheral surface topography can be used to identify a substantially complementary harvesting location. In some embodiments, the elongated rod members 320″ can then be retracted into the mechanical housing 310″ in preparation for harvesting the implant. The cutting member 316″ can then be implemented to harvest an implant, e.g., a graft, from a harvesting site.

As would be understood by those of ordinary skill in the art, the dimensions of the cutting member 316″ generally establish the desired height, i.e., depth, of the implant to be harvested. Thus, with the cutting member 316″ exposed, the graft harvesting device 300″ can be driven into a donor location until the bottom surface of the retracted broach 318″ abuts the top surface of the harvested implant. The graft harvesting device 300″ can then be retracted from the donor location with the harvested implant within the cavity of the cutting member 316″. In particular, once an implant has been harvested, the desired portion of the implant generally resides inside the cavity of the cutting member 316″ against the bottom surface of the retracted broach 318″, while the undesired portion of the implant generally extends from the cutting member 316″ for trimming.

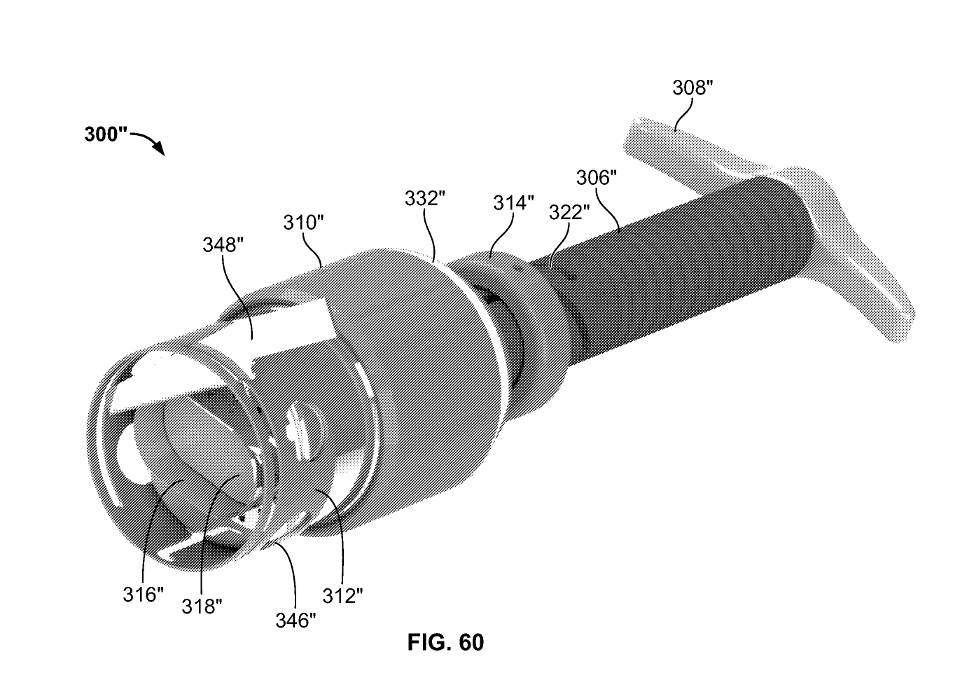

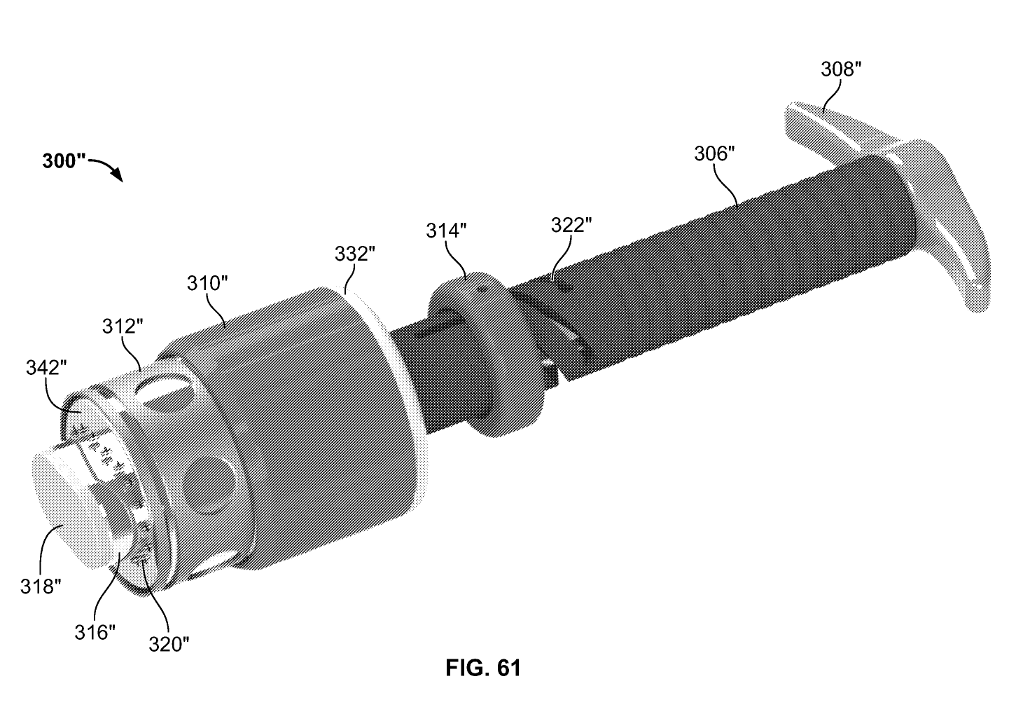

As illustrated in FIG. 60, in some embodiments, the trimmer guide 312″ can be lowered such that the trimmer path 346″ is substantially aligned with the lowest surface of the cutting member 316″. In some embodiments, the cutting member 316″ and the broach 318″ can be retracted further into the mechanical housing 310″ to substantially align the lowest surface of the cutting member 316″ with the trimmer path 346″. Thus, the position of the cutting member 316″ can be adjusted by the user for various implant dimensions. The trimmer path 346″ generally maintains a substantially planar trimming surface during the trimming procedure. A trimmer 348″, e.g., a saw, a blade, and the like, can be manually or electronically implemented to trim the undesired portion of the implant (not shown) extending from the lowest portion of the cutting member 316″. Once the implant has been trimmed to the desired depth, the broach 318″ can be extended out of the cutting member 316″, as shown in FIG. 61, to eject the implant from the graft harvesting device 300″. The substantially planar bottom surface of the broach 318″ ensures an even force distribution against the implant surface defining the desired topography, thereby reducing or eliminating the risk of damaging the implant during ejection.

Although illustrated as a unitary structure, in some embodiments, the graft harvesting device 300″ can be configured as a disposable section and a reusable section. For example, in some embodiments, the cutting member 316″, the broach 318″, the elongated rod members 320″, the locking mechanism 336″, the fixation plate 342″, the trimmer guide 312″, the mechanical housing 310″, the lower flange 338″, the spring 344″, and the fixation element 332″ can be configured as interchangeable components to allow a user to vary the configuration and dimensions of the implant being harvested. The remaining components of the graft harvesting device 300″ can be reused to reduce the costs associated with the surgical procedures discussed herein. For example, the disposable or interchangeable components can be threaded against complementary threads located on a distal end of the handle 306″ for mechanically interlocking the disposable or interchangeable components relative to the reusable components.

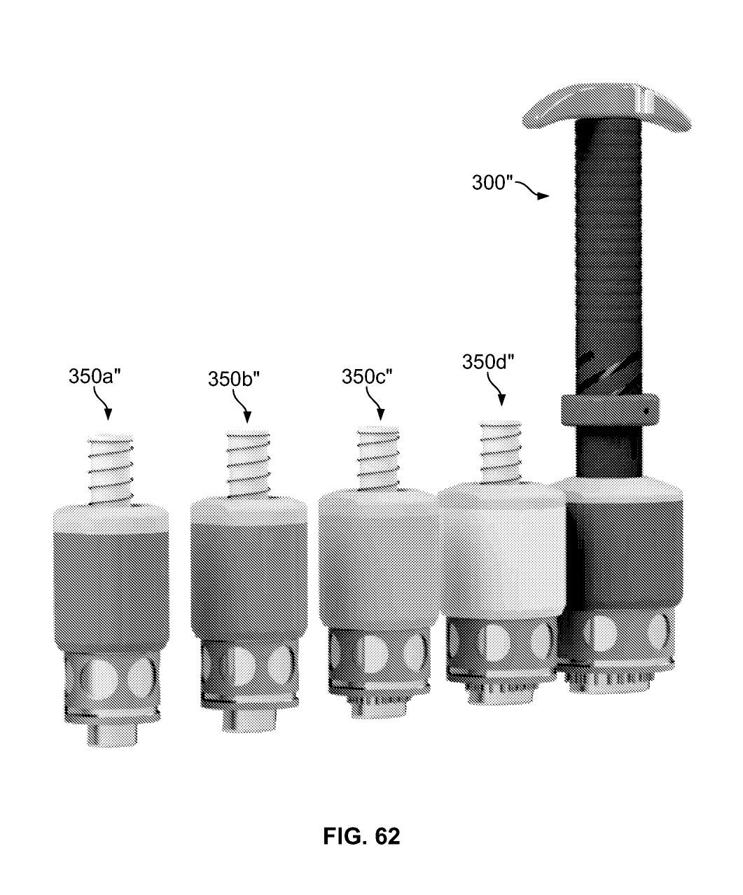

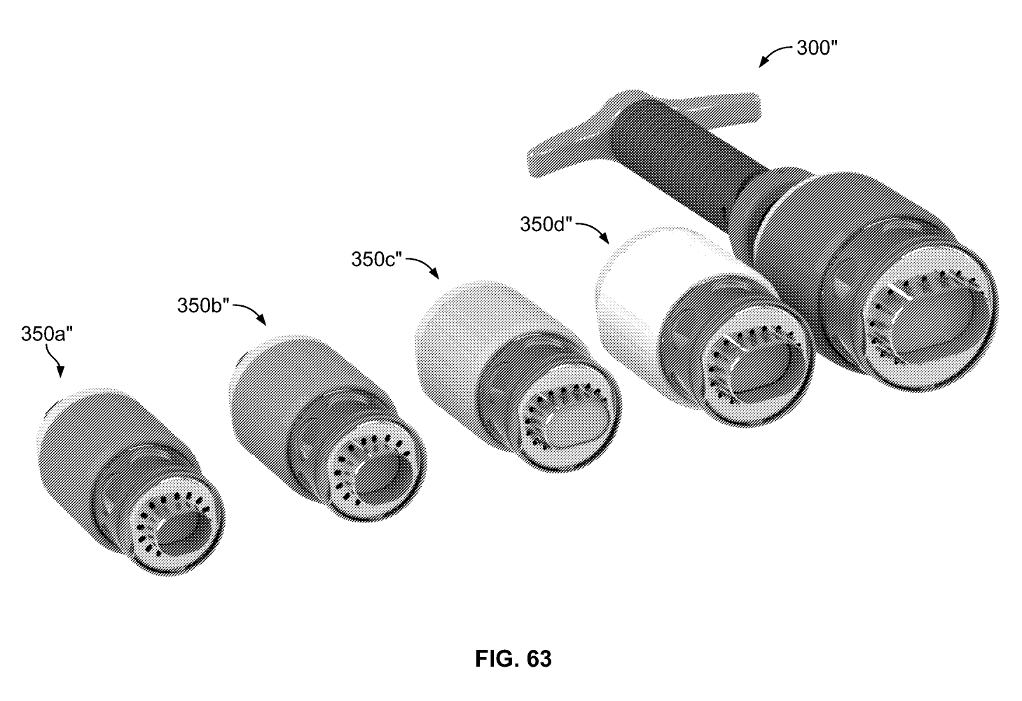

For example, FIGS. 62 and 63 illustrate an exemplary graft harvesting device 300″ with alternatively sized and interchangeable sections 350a″-350d″. The interchangeable sections 350a″-350d″ can vary depending on, e.g., the cutting member 316″ and/or broach 318″ size and configuration being implemented based on the defect region cavity 302″. As discussed above, the exemplary interchangeable sections 350a″-350d″ can generally be mechanically interlocked with the graft harvesting device 300″ at an interlocking mechanism, e.g., the complementary threads between the fixation element 332″ and the handle 306″.

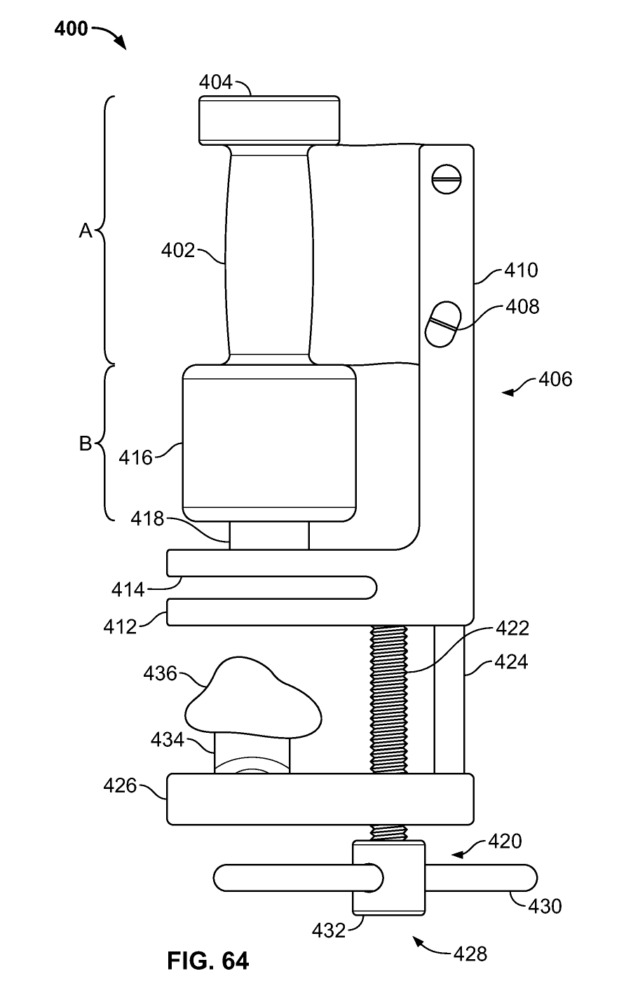

With reference to FIG. 64, an alternative exemplary embodiment of a graft harvesting device 400 is presented. The graft harvesting device 400 generally includes a reusable portion A and a disposable portion B. The reusable portion A includes a handle 402 defined by an elongated shaft therein, a top cap 404 fixed to the handle 402, and an integral cutter guide 406 fixed to the handle 402. In some embodiments, the reusable portion A can include a hammer mechanism substantially similar to the hammer mechanism 312 of FIG. 46. The integral cutter guide 406 can be configured as an attachment member 408, e.g., a hinge, a connecting member 410, a cutter guide head 412 and a cutter guide channel 414. It should be understood that the connecting member 410 can be configured as a telescoping connecting member 410, thus permitting a user to vary the length of the connecting member 410 as needed depending on the configurations and/or dimensions of the disposable portion B components. The graft harvesting device 400 can include a spring-loaded button (not shown) which can be actuated to mechanically control the disposable portion B components. The reusable portion A components can be detachably secured, e.g., mechanically interlocked, to the disposable portion B components through a mechanical connection.

The disposable portion B of FIG. 64 generally includes a cutting member assembly 416 which can include, e.g., a cutting member 418, a plurality of elongated rod members, a locking mechanism, a broach, and the like, which function substantially similarly to the previously discussed cutting member assemblies. In some embodiments, the graft harvesting device 400 can include an exemplary crank-actuated mechanism 420 mounted with respect to the reusable portion A. The exemplary mechanism 420 generally includes a threaded rod 422, an alignment rod 424, a platform 426 and an actuator 428. The actuator 428 includes handles 430 which can be axially rotated to move the actuator body 432 down the threaded rod 422 and against the platform 426. The actuator body 432 thereby imparts a driving force against the platform 426 and drives the platform 426 in the direction of the cutting member 418. As the platform 426 is translated in the direction of and/or away from the cutting member 418, the alignment rod 424 can travel within a complementary channel (not shown) in, e.g., the integral cutter guide 406, to maintain an aligned translation of the platform 426 relative to the cutting member 418.

The platform 426 can include thereon a fixation component 434 for fixating donor cartilage 436 onto the platform 426. Thus, donor cartilage 436 can be fixated to the platform 426 and the actuator 428 can be actuated to impart a force against the platform 426 to drive the donor cartilage 436, e.g., an allograft, to a desired depth in the cutting member 418. When the donor cartilage 436 has been driven into the cutting member 418 to a desired depth, the platform 426 can be translated away from the cutting member 418, leaving the harvested implant within the cutting member 418. In particular, the desired implant can remain within the cutting member 418 cavity, while the undesired portion of the harvested implant can protrude out of the distal end of the cutting member 418 for trimming. A cutting device, e.g., a saw, can then be used to trim the donor cartilage 436 along the cutter guide channel 414 and the implant can be ejected out of the cutting member 418.

In some embodiments, the graft harvesting device 400 can be detachably secured to a stable surface, e.g., an operating table, during the cartilage harvesting procedure. In some embodiments, the crank-actuated mechanism 400 can be used to initially insert and fixate the donor cartilage 436 in the cutting member 418 until a steady position and/or orientation has been established and the hammer mechanism can then be implemented to drive the cutting member 418 to the full desired depth in the donor cartilage 436. The exemplary crank-actuated mechanism 420 generally ensures the proper orientation, e.g., positioning, angle, and the like, of the graft harvesting device 400 relative to the donor cartilage 436 and/or the captured surface topography surrounding the defect region cavity and can minimize the potential toggle effect of the hammer mechanism.

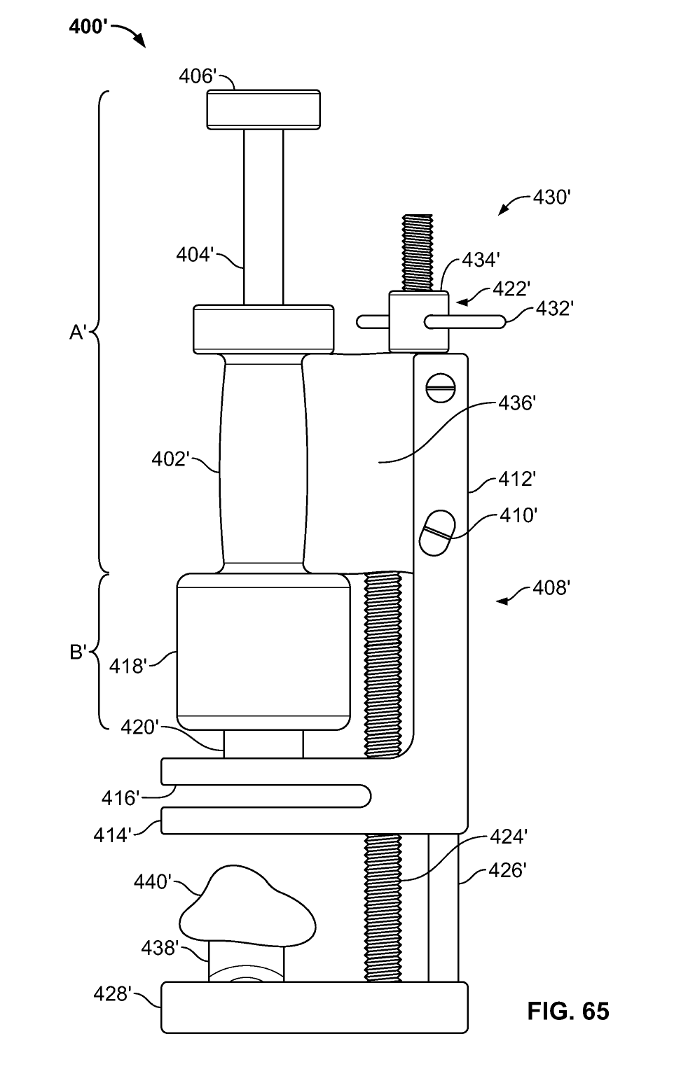

With reference to FIG. 65, an alternative exemplary embodiment of a graft harvesting device 400′ is presented. The graft harvesting device 400′ can be substantially similar to the graft harvesting device 400 of FIG. 64, except for the configuration of the crank-actuated mechanism 422′. The graft harvesting device 400′ generally includes a reusable portion A′ and a disposable portion B′. The reusable portion A′ includes a handle 402′ defined by an elongated shaft 404′ therein, a top cap 406′ fixed to the shaft 404′, and an integral cutter guide 408′ fixed to the handle 402′. In some embodiments, the handle 402′ can translate along the shaft 404′ to act as a hammer mechanism substantially similar to the hammer mechanism 312 of FIG. 46. The integral cutter guide 408′ can be configured as an attachment member 410′, e.g., a hinge, a connecting member 412′, a cutter guide head 414′ and a cutter guide channel 416′. It should be understood that the connecting member 412′ can be configured as a telescoping connecting member 412′, thus permitting a user to vary the length of the connecting member 412′ as needed depending on the configurations and/or dimensions of the disposable portion B′ components. The graft harvesting device 400′ can include a spring-loaded button (not shown) which can be actuated to mechanically control the disposable portion B′ components. The reusable portion A′ components can be detachably secured, e.g., mechanically and/or electrically interlocked, to the disposable portion B′ through a mechanical connection.

The disposable portion B′ of FIG. 65 generally includes a cutting member assembly 418′ which can include, e.g., a cutting member 420′, a plurality of elongated rod members, a locking mechanism, a broach, and the like, which function substantially similarly to the previously discussed cutting member assemblies. The graft harvesting device 400′ also includes an exemplary crank-actuated mechanism 422′ mounted with respect to the reusable portion A′. The crank-actuated mechanism 422′ generally includes a threaded rod 424′, an alignment rod 426′, a platform 428′ and an actuator 430′. The actuator 430′ includes handles 432′ which can be axially rotated to move the actuator body 434′ down the threaded rod 424′ and against a support structure 436′ associated with either the reusable portion A′ components and/or the connecting member 412′. The actuator body 434′ thereby imparts a driving force against the support structure 436′ to drive the platform 428′ in the direction of the cutting member 420′. In some embodiments, rather than driving the platform 428′ in the direction of the cutting member 420′, the actuator body 434′ can impart a driving force against the support structure 436′ such that the cutting member 420′ is driven in the direction of the platform 428′. As the platform 428′ and/or the cutting member 420′ travel relative to each other, the alignment rod 426′ can travel within a complementary channel (not shown) in, e.g., the integral cutter guide 408′, to maintain an aligned translation of the platform 428′ and/or the cutter member 420′ relative to each other.

The platform 428′ can include thereon a fixation component 438′ for detachably fixating a donor cartilage 440′ onto the platform 428′. Thus, a donor cartilage 440′ can be fixated to the platform 428′ and the actuator 430′ can be actuated to impart a force against the support structure 436′ to drive the donor cartilage 440′, e.g., an allograft, to a desired depth in the cutting member 420′ or drive the cutting member 420′ into the donor cartilage 440′. When the donor cartilage 440′ has been driven into the cutting member 420′ to a desired depth, or vice versa, the cutting member 420′ can be retracted from the remaining donor cartilage 440′, while housing the harvested implant within the cutting member 420′ and the undesired cartilage protruding from a distal end of the cutting member 420′ for trimming A cutting device, e.g., a saw, can then be used to trim the donor cartilage 440′ along the cutter guide channel 416′ and the implant can be ejected out of the cutting member 420′.

In some embodiments, the graft harvesting device 400′ can be detachably secured to a stable surface, e.g., an operating table, during the cartilage harvesting procedure. In some embodiments, the crank-actuated mechanism 422′ can be used to initially insert and fixate the donor cartilage 440′ in the cutting member 420′, or vice versa, until a steady position and/or orientation has been established and the hammer mechanism can then be implemented to drive the cutting member 420′ to the full desired depth in the donor cartilage 440′. The exemplary crank-actuated mechanism 422′ generally ensures the proper orientation, e.g., positioning, angle, and the like, of the graft harvesting device 400′ relative to the donor cartilage 440′ and/or the captured surface topography surrounding the defect region cavity and can minimize the potential toggle effect of the hammer mechanism.

Although exemplary instruments, methods and/or systems have been described herein as including elongated pin members for capturing surface topographies of the defect region, areas surrounding the defect region and/or donor sites, it should be understood that the features and/or functions of the disclosed instruments, methods and/or systems can be advantageously implemented independent of the elongated pin member topography capture functionality. Thus, for example, the trial members, templates and/or harvesting instruments discussed herein can be advantageously used for orthopedic applications without the elongated pin members for capturing surface topography related to the defect region and/or the donor site.

In accordance with yet another embodiment of the present disclosure, a method for defect repair is provided, generally including the steps of establishing a referential orientation of an instrument relative to an anatomical location, capturing a partial or an entire surface topography of the anatomical location of the defect region, forming a defect region cavity of a predefined geometry in the anatomical location, and using the captured surface topography of the anatomical location of the defect region to identify a donor location with a complementary surface topography as a harvest region for a plug to fill the defect region cavity. The defect region cavity can generally be formed with a predefined depth and can be formed at a substantially right angle relative to the axis of the instrument used to form the defect region cavity.

The exemplary method generally further includes using a detachable broach member for cleaning the defect region cavity and using a plurality of elongated rod members for capturing a peripheral surface topography of the anatomical location in proximity to the defect region cavity. Further still, the exemplary method generally includes obtaining a plug from the harvest region, using a cutter guide to trim the plug to a predefined depth, using a detachable broach member to eject the plug from a cutter, and introducing the plug into the defect region cavity. In general, the defect region cavity can be formed using a template having a predefined opening geometry, the plug can be obtained using the cutter having a cutting geometry, and the predefined opening geometry of the template and the cutting geometry of the cutter correspond to each other.

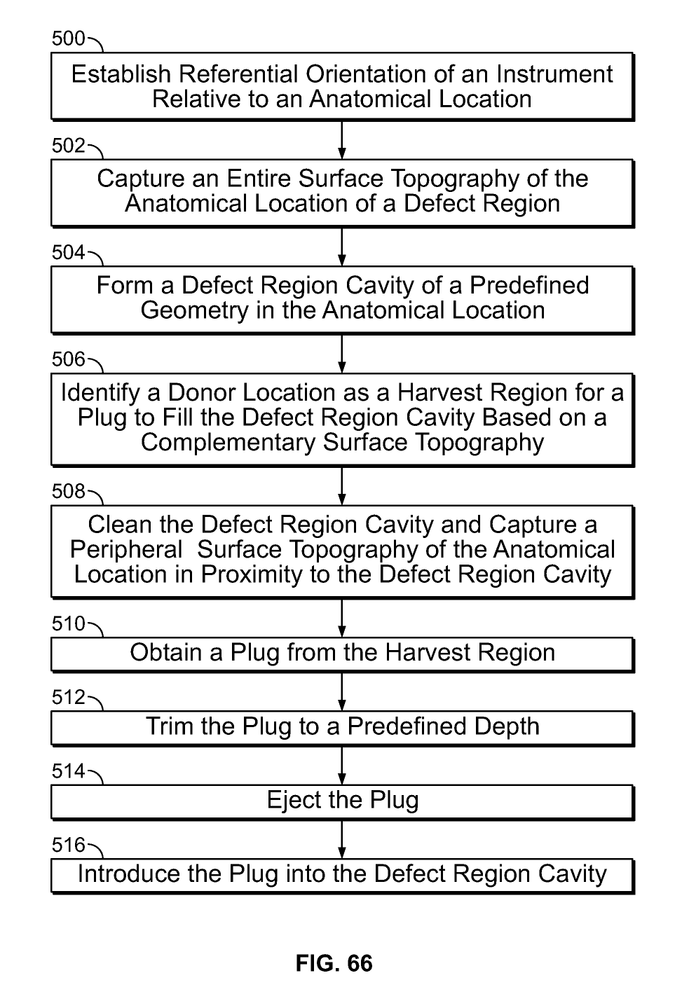

Turning now to FIG. 66, a flowchart is provided of an exemplary method for defect repair implementing the exemplary trial members and graft harvesting members discussed herein. In particular, the exemplary method includes establishing a referential orientation of an instrument to be implemented in conjunction with the exemplary trial members and graft harvesting members relative to an anatomical location (500). A trial member can be utilized to capture a partial or an entire surface topography of the anatomical location of a defect region (502). A defect region cavity can be formed with a predefined geometry in the anatomical location (504). A donor harvesting location can be identified as an appropriate harvest region for a donor graft plug to fill the defect region cavity based on a complementary surface topography (506). The defect region cavity can be cleaned and simultaneously the peripheral surface topography of the anatomical location in proximity, e.g., surrounding, the defect region cavity can be obtained (508). In some embodiments, steps 506 and 508 can be reversed such that the defect region cavity can be cleaned and the peripheral surface topography of the anatomical location surrounding the defect region cavity can be obtained. The captured peripheral surface topography can then be used to identify a donor location with a complementary surface topography. A donor graft plug with a complementary entire and/or peripheral surface topography can be obtained from a harvest region (510). The donor graft plug can be trimmed to a predetermined depth/height to property fill the defect region cavity (512). The donor graft plug can be ejected out of the cutting member of a graft harvesting device (514). The donor graft plug can then be introduced into the defect region cavity (516).

As discussed above, the exemplary instruments, methods and systems may be used in connection with mapping techniques and systems discussed in PCT applications entitled “Systems, Devices and Methods for Cartilage and Bone Grafting” and “Instruments, Methods and Systems for Harvesting and Implanting Cartilage Materials,” which published as WO 2009/154691 A9 (corrected version) and WO 2011/008968 A1, respectively, which have been previously incorporated herein by reference. Thus, in exemplary embodiments of the present disclosure, a clinician may be guided in his use of the disclosed instruments and systems by cartilage surface mapping data in locating/identifying harvest sites for “best fit” grafts, i.e., grafts that exhibit desired geometric and/or surface attributes for use in particular implantation site(s). Alternatively, the disclosed instruments, methods and systems may be employed to access anatomical sites independent of such mapping techniques/systems.

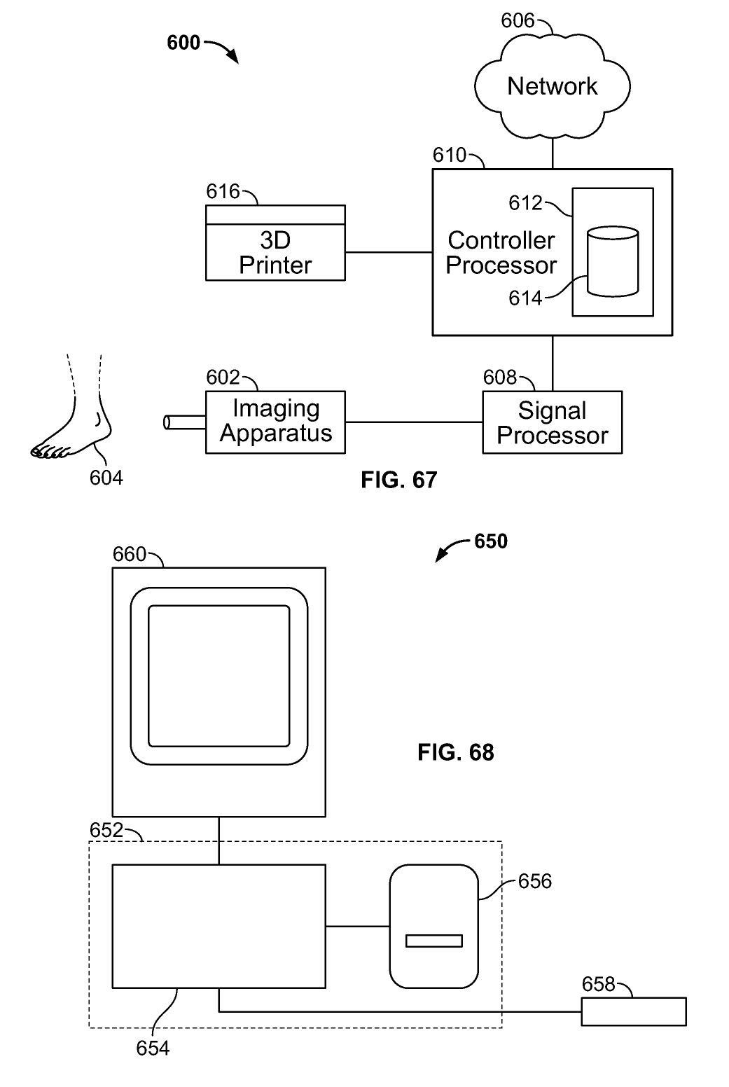

With reference to FIG. 67, an exemplary schematic diagram is shown of a surface mapping system 600 to acquire data regarding cartilage and/or bone anatomies and to enable identification of suitable donor sites to harvest cartilage to repair defects in a patient's bone. The exemplary system 600 generally includes an imaging apparatus 602 to capture image data of an area on a patient's body, e.g., a patient's foot 604, which includes at least one of the defect regions and an area around the defect. The imaging apparatus 602 includes, e.g., one or more of a Magnetic Resonance Imaging (MRI) system, a computed tomography apparatus configured to generate three-dimensional images from a series of two-dimensional images (e.g., X-Ray) taken around a single axis of rotation, a Medical sonography (ultrasound) imaging device, and any other suitable imaging device to acquire data representative of anatomical structures in a patient's body. In some exemplary embodiments, the data relating to the defect region of the patient may have been acquired at an earlier time and/or at some location other than where the system 600 is located, in which case, the data can be received at the system 600 from some remote location which can electronically communicate, e.g., via one or more types of communication networks such as a network 606, including the Internet, a telephony network, and the like, the data relating to the defect region of the patient.

The imaging apparatus 602 can acquire one or more images of a site in the body of a patient who has the defect, e.g., the talus surface at the foot 604, requiring a cartilage-bone graft procedure to correct. In some exemplary embodiments, the mapping system 600 also includes an optional signal processing unit 608 connected to the imaging apparatus 602. The processing unit 608 receives the signals communicated from the imaging apparatus 602 and performs signal processing and/or enhancement operations. Signal enhancement operations may include, e.g., amplification, filtering, and the like. For example, the processing unit 608 can be configured to perform noise reduction to remove noisy artifacts from acquired image data. Other types of processing can include image processing operations to transform the image data into resultant data which can be more easily manipulated for the purpose of identifying donor sites. For example, the acquired data can be processed to generate surface model corresponding to the defect region and/or the area proximate the defect region, transform spatial representations into another domain, e.g., the frequency domain, which is more conducive for various type of processing, and the like.

The processed data can subsequently be communicated to the controller processor 610. The controller processor 610 includes a storage device 612 to store the data (processed and/or raw acquired data) relating to the defect region of the patient, and to store a donor database 614 which includes information on each of a plurality of donor sites of the body. As will become apparent below, the database 614 can be constructed based on data acquired from multiple sources and/or multiple specimens. The acquired data can be used to develop and/or expand the database 614 and enhance the sensitivity and specificity of the system 600. Typically, the data stored on the database 614 pertains to healthy, non-injured specimens (or a composite representation thereof), thus enabling identification of suitable healthy sites in the body from which bone and/or cartilage can be harvested to perform bone-cartilage grafts. In some embodiments, the data stored on the database 614 pertains to defect regions, thus enabling identification of suitable defect region cavities which are compatible with the available harvest locations. In some embodiments, the data stored on the database 614 can pertain to both healthy, non-injured specimens and defect regions of various patients. The controller processor 610 can thereby be configured to receive a first data relating to a defect region of a patient and to identify, based on the received first data, at least one donor site from the donor database 614 from which a graft of bone and cartilage to repair the defect region of the patient can be harvested.

In some embodiments, the storage device 512 hosting the donor database 614, or another storage device hosting the database 507, can be located at one or more remote locations which can be accessed by multiple systems, such as the mapping system 600. Thus, such a remote device can serve as a central data repository on which data pertaining to donor sites may be stored. A user locally interacting with the system 600 can therefore access remotely, via a network 606, a database such as the database 614 to retrieve data as required. For example, and as will be described in greater details below, data pertaining to potential donor sites which is compared to data relating to a defect region can be retrieved from a remote location. Optionally, a 3D printer 616 can be locally or remotely interconnected to the controller processor 610. Such a 3D printer 616 can be used to create 3D custom templates corresponding to any identified donor site and/or to the defect region.

In some implementations, the controller processor 610 can also be configured to perform learning functions. A machine learning system is generally a system which iteratively analyzes training input data and the input data's corresponding output, and derives functions or models that cause subsequent inputs to produce outputs consistent with the machine's learned behavior. Thus, in some embodiments, the controller processor 610 can be configured to perform learning functions which include, e.g., identifying the type of donor site corresponding to newly received data, classifying the data so it is associated with other data sets corresponding to the same anatomical locations, automatically selecting several potentially suitable donor sites for further processing with respect to data received regarding the defect region, and the like. Some implementations of learning functionalities may be performed using, e.g., a neural network system implementation. A neural network includes interconnected processing elements (effectively the system's neurons), whose connections can be varied, thus enabling the neural network to adapt (or learn) in response to training data it receives. In some embodiments, a learning system may be implemented using decision trees, e.g., a graph of decisions/actions and their possible outcomes. A decision tree takes as input an object or situation described by a set of properties, and outputs a decision, i.e., an outcome. Alternatively and/or additionally, in some embodiments, the learning system may be implemented using regression techniques. Regression techniques produce functions, e.g., curves, which best fit a given set of data points. These curves can subsequently be applied to input data to determine the output based on the derived curves. Derivation of best fit curves is typically the solution to optimization problems, in which a particular error measure, e.g., least-square error, is being minimized Other types of learning system implementations may also be used.

With reference to FIG. 68, a schematic diagram of a generic computing system 650 is provided which can be used to implement the controller processor 610 and/or the signal processing unit 608. The computing system 650 includes a processor-based device 652, e.g., a personal computer, a specialized computing device, and the like, which typically includes a central processor unit (CPU) 654. In addition to the CPU 654, the system 650 includes main memory, cache memory and bus interface circuits (not shown). The processor-based device 652 includes a mass storage element 656, which may be the same device or a separate device from the storage device 612. The mass storage element 656 can be, e.g., a hard drive associated with personal computer systems. The computing system 650 may further include a keyboard 658 and a monitor 660, e.g., a cathode ray tube (CRT) or liquid crystal display (LCD) monitor.

The processor-based device 652 can be configured to facilitate, e.g., the implementation of the data capture and/or mapping operation used to identify suitable donor sites for harvesting a graft of bone-cartilage as described herein. The storage device 656 may thus include a computer program product which, when executed on the processor-based device 652, performs operations to facilitate the implementation of the data capture, mapping and/or site identification procedures described herein. The processor-based device 652 may further include peripheral devices to enable input/output functionality, e.g., a CD-ROM drive, a flash drive, a network connection, and the like, for downloading related content to the connected system. Such peripheral devices may also be used for downloading software containing computer instructions to enable general operation of the respective system/device, as well as data from remote locations, e.g., donor site data. Alternatively and/or additionally, in some exemplary embodiments, special purpose logic circuitry, e.g., a field programmable gate array (FPGA) or an application-specific integrated circuit (ASIC) may be used in the implementation of the system 650. Other modules which may be included with the processor-based device 652 are speakers, a sound card, a pointing device, e.g., a mouse, a trackball or a touch-based graphical user interface (GUI), by which the user can provide input to the computing system 650, and the like. The processor-based device 652 may include an operating system, e.g., Windows XP® Microsoft Corporation operating system. Alternatively, other operating systems could be used. Additionally or alternatively, one or more of the procedures performed by the signal processor 608 and/or the controller processor 610 may be implemented using processing hardware, such as digital signal processors (DSP), field programmable gate arrays (FPGA), mixed-signal integrated circuits, and the like.

The various systems and devices constituting the system 600 may be connected using conventional network arrangements. For example, the various systems and devices of system 600 may constitute part of a public private packet-based network, e.g., the Internet. Other types of network communication protocols may also be used to communicate between the various systems and devices. Alternatively, the systems and devices may each be connected to network gateways which enable communication via a public network, such as the Internet. Network communication links between the systems and devices of system 600 may be implemented using wireless or wire-based links. For example, in some embodiments, the controller processor 610 may include a communication apparatus, e.g., an antenna, a satellite transmitter, a transceiver such as a network gateway portal connected to a network, and the like, to transmit and/or receive data signals. Further, dedicated physical communication links, such as communication trunks, may be used. Some of the various systems described herein may be housed on a single processor-based device, e.g., a server, configured to simultaneously execute several applications.

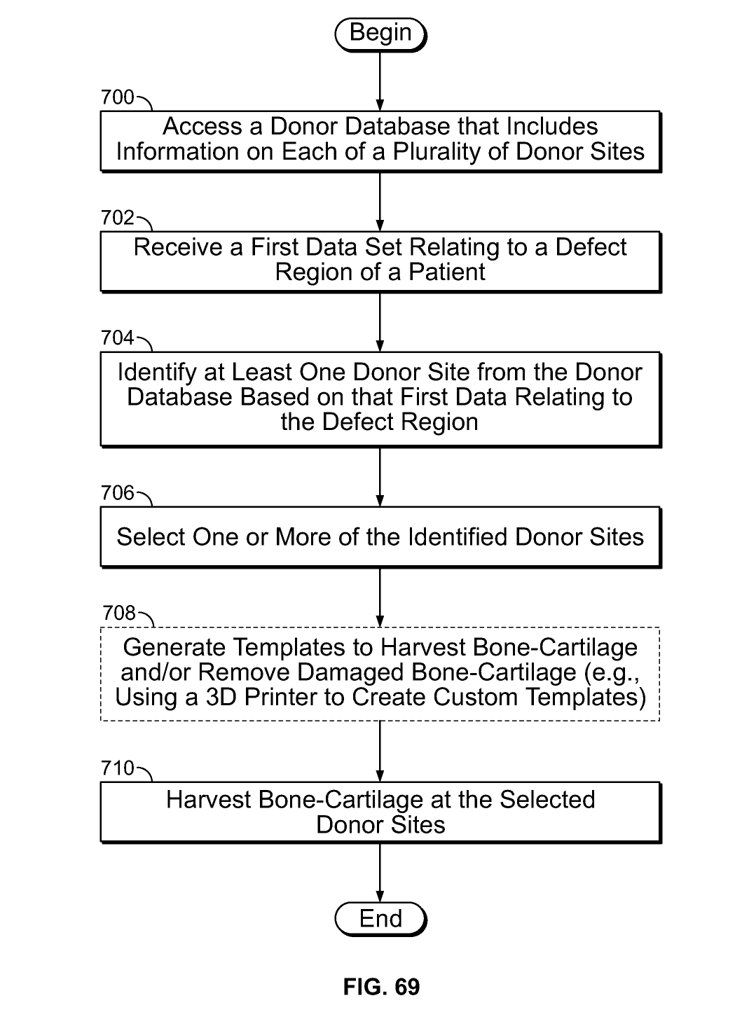

Referring to FIG. 69, a flowchart of a procedure to identify suitable donor sites for bone-cartilage grafts for repairing a defect region of a patient is shown. Initially, a computing device, such as a computer or the controller processor 610 depicted in FIG. 67, accesses a donor database 614 which includes information on each of a plurality of donor sites that may have been compiled and/or evolved from several sources of data (700). In some exemplary embodiments, the database 614 may have been populated with data downloaded, or otherwise retrieved, from remote locations which maintain data regarding potential donor sites. In some exemplary embodiments, the data may have been obtained by acquiring raw data, e.g., image data obtained using conventional imaging techniques, such as MRI imaging, CT imaging, ultrasound imaging, laser scans, and the like, from specimens having healthy cartilage of specific anatomical locations, e.g., joints which do not have defects or are otherwise non-injured. For example, in some embodiments, data acquired using a large sample of individuals may be used to assemble data about possible donor sites in those individuals, including data representative of the topology, health and other physiological attributes, e.g., gender, age, race, activity index, BMI, V02, cartilage thickness, bone density, and the like, of those donor sites. Such data acquired using such a sample of individuals may include data about some or all feasible sites in a body from which bone and/or cartilage may be harvested. Accordingly, the individuals used to acquire this data may be put through a comprehensive and systematic protocol of data acquisition procedure such that data regarding all (or substantially all) possible donor sites is acquired.

For example, the data acquisition stage required for constructing the database may require that all the joint areas in a person's feet be imaged using one or more imaging devices and/or surveyed using non-imaging type devices, e.g., devices utilized to measure bone density, to obtain an accurate and comprehensive database 614. Data processed in this manner can be added to the donor database 614. As noted, in some embodiments, a learning system, e.g., implemented on the controller processor 610 or on some other dedicated processing device, may be used to process acquired data of graft sites (e.g., donor sites and/or recipient sites) which is to be added to the database 614. For example, such a learning system may be used to determine (through implemented classification functions) the identity of the site with respect to which data was received, facilitate the identification procedure to identify donor sites which would be suitable for harvesting bone-cartilage to repair the particular damaged site, and the like.

The donor sites with respect to which data is acquired and added to the database 614 include donor sites of different shapes and sizes, including donor sites suitable for harvesting non-cylindrical bone-cartilage grafts. The data for those donor sites can subsequently be used to identify suitable donor sites from which cylindrical and non-cylindrical bone-cartilage grafts can be harvested. For example, the systems described herein enable matching irregularly shaped defects of the damaged/injured recipient site(s) to available donor sites which can be used to harvest non-cylindrical bone-cartilage grafts. Conventional bone-cartilage grafting systems and methods typically extract grafts having standard shapes, e.g., cylindrical, thus limiting the repertoire of available donor sites, e.g., donor sites from which such standard shaped grafts can be harvested. Once suitable donor site are identified, various types of grafts can be harvested, including standard-shaped grafts, e.g., cylindrical grafts, as well as irregularly-shaped grafts. Harvesting irregularly shaped grafts can be performed using a set of predetermined irregularly shaped templates or, in some exemplary embodiments, by generating custom templates.

In some exemplary embodiments, the specimens used to acquire data to populate the donor site database 614 may include cadavers. Under those circumstances, more invasive data acquisition procedures may be used to acquire the data. For example, in some embodiments, one or more of a cadaver's joints may be disarticulated to expose the actual cartilage tissue. With the joint sites of the cadavers disarticulated, a high resolution image scanner may be used to scan the tissue to obtain an accurate representation of the cartilage tissue. A suitable laser scanner to scan exposed cartilage may be, e.g, a NextEngine 3D Scanner manufactured by NextEngine, Inc. Other laser scanners and/or other types of high quality image capture devices may be used.

In some exemplary embodiments, data acquired from multiple specimens, e.g., live individuals and/or cadavers, may be used to generate a composite representation of donor sites. For example, the data acquired may be averaged to obtain a general representative model of the plurality of donor sites. In some variations, several representative models of donor sites and their associated data may be generated from multiple specimens that each correspond to a particular individual type such that, when identification of a suitable donor site is undertaken, a model which is more representative of the particular traits of the patient for whom a bone-cartilage graft is required can be used. For example, different general model sets of donor sites may be constructed for male and female models.

In circumstances where the database 614 is constructed, at least partly, by collecting data about donor sites (and areas surrounding such donor sites) from specimens, a system arrangement similar to the arrangement depicted in FIG. 67 may be used. Thus, such an arrangement would include an imaging apparatus 602, e.g., a NextEngine laser scanner, to capture data. The captured data would be forwarded to a signal processing unit 608, which may be implemented as a processor-based computing device to perform digital processing, e.g., filtering, on the data and/or a dedicated processing device to perform some or all of the processing operations. A storage device 612 to store captured and/or processed data may also be provided. In some embodiments, such storage device 612 can be locally connected to a processor-based computing device which may also serve to perform data processing, perform database management operations, e.g., by executing database management tools, and to perform the donor site identification procedure to identify suitable donor sites from which bone-cartilage may be harvested.

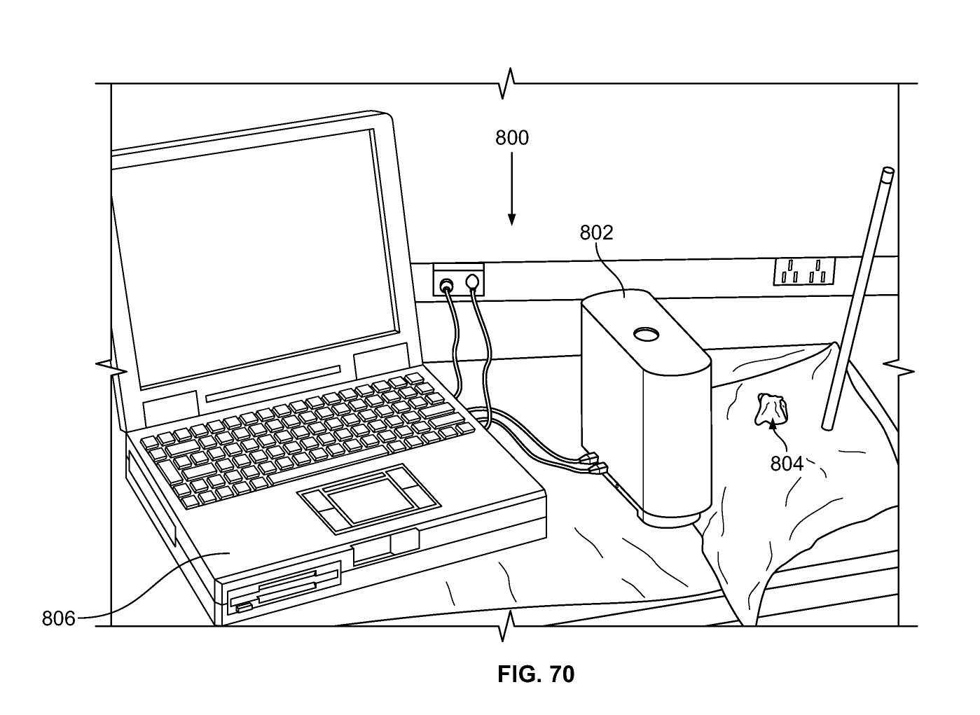

With reference to FIG. 70, an exemplary arrangement of a system 800 to acquire, process and/or store data is shown. The system 800 generally includes a laser scanner 802, e.g., a NextEngine scanner, whose imaging port, i.e., an outlet through which laser radiation is directed at the object being scanned, is facing an object 804, e.g., a disarticulated body joint. Data acquired by the imaging apparatus 802 can be communicated to a processing apparatus 806, e.g., a computer. The processing apparatus 806 typically includes software implemented applications to interface and/or interact with imaging apparatus 802 and may perform preliminary processing on data communicated by the imaging apparatus 802, e.g., perform analog-to-digital conversion, down-sample the data, and the like. The processing apparatus 806 may also run software-based implementations of data processing applications, e.g., SolidWorks 3D CAD software applications, and the like. Further, the processing apparatus 806 may include software implementations to perform the donor site identification procedure described herein. Data captured and processed may be maintained in a database implemented on the processing apparatus 806 or may be stored on a remote storage device and processing center implemented, for example, on a remote server connected to the processing apparatus 806 via a communications network.

Data acquired by imaging apparatus 802 for populating the donor site database may be processed to, for example, remove noisy artifacts from the image, remove unnecessary data, perform various mathematical mapping and/or transformation operations (e.g., normalization operations, re-sizing/scaling operations so all data corresponds to features at the same scale, frequency domain transformations, and the like) to transform the data into formats which are more conducive for subsequent search operation on the database. As noted, further processing on the image data (including image data on which some preliminary processing such as noise filtering and/or artifact removals have already been performed) can be performed on the data to convert it into a format which can subsequently be more easily controlled and can be more conducive for performing the donor site identification procedure described herein, e.g., using a format which enables comparisons of different donor site surfaces to one another. In some implementations, the data acquired can be used to generate surface models representative of the donor sites. The surface model may include data regarding the topology of the area, as well as other information descriptive of the area, e.g., bone thickness, bone density, and the like.

Several procedures may be used to generate the surface models. For example, in some exemplary embodiments, the captured data of the defect region can be provided as input to various computer aided design (CAD) interface applications, e.g., the SolidWorks 3D CAD application developed by Dassault Systemes SolidWorks Corp., and the like, such that the application generates a 3D rendering corresponding to the data provided. Specifically, the point cloud of data representative of an acquired image can be incorporated into SolidWorks (or any other CAD application used) to generate a resultant surface model. This data can then be stored in a format compatible with the graphical representation rendering or may be converted and stored using another type of representation of the surface model features, e.g., a representation of a composite of graphical primitives corresponding to, for example, dimensions and curvatures of lines or segments of the surface model, and the like. The generated surface model may be compared with, for example, a surface model representative of the damaged cartilage/bone of a defect region, to determine if the potential donor site would be suitable for harvesting bone-cartilage to repair the defect region of the patient. Surface comparisons may be performed visually by the operator of the system, e.g., a surgeon, who examines the surfaces compared to each other and selects one position/orientation which appears to result in the best match, or via a processing device. The procedure of matching the model surface of the defect region to model surfaces of potential donor sites can be repeated for other donor/recipient sites.

In some exemplary embodiments, the generated surface model of the donor site may be further manipulated to fit the surface model into a corresponding bone structure to provide further details on the anatomical structure of the potential donor site and provide orientation context to the user on how the surface model is overlaid relative to the bone structure. In some embodiments, the model representation of the bone structure on which the cartilage surface model is overlaid may have been acquired from other specimens, i.e., not necessarily from the same individual whose cartilage data was acquired, using an imaging device, such as an MRI imaging apparatus, a CT imaging apparatus and/or a laser scanner. Under such circumstances, when a generated surface model of the cartilage is overlaid on a previously acquired or imported model of the bone structure, small anatomical differences between the two models may be evident, e.g., topographical differences, size differences, and the like. Alternatively and/or additionally, in some embodiments, the bone structure models and the cartilage models may have been derived from the same set of specimens.

With reference again to FIG. 69, the procedure further includes receiving a first data set relating to a defect region of a patient (702). As noted, the defect region includes an area of a bone, a portion of which includes at least one of a missing and/or damaged cartilage, e.g., hyaline cartilage, and the like. In some embodiments, the data relating to the defect region may be representative of at least one of, for example, the defect region and the area around the defect region. Such data may be acquired by using imaging techniques, such as MRI, CT, ultrasound, and the like, as noted previously, to image the area and construct, using the data, a surface model. The data may have been sent via a communications link by a health professional, such as the patient's physician or the surgeon who will perform the graft. The surface model may include data regarding the topology of the area, as well as other information descriptive of the area, e.g., bone thickness, bone density, and the like. As noted with respect to the procedure for generating surface models for the potential donor sites, several procedures may be used to generate the surface model, including, e.g., using the Pro/Engineer CAD application developed by Parametric Technology Corporation, MA, the SolidWorks 3D CAD application developed by Dassault Systemes SolidWorks Corp., and the like, to generate a 3D rendering corresponding to the received first data corresponding to the defect region and the area surrounding it.

As further noted, the data can then be stored in a format compatible for providing graphical representations of the rendering or may be converted and stored as numerical representations of the surface model features, e.g., be represented as primitives corresponding to dimensions and curvatures of lines or segments of the surface model. Based on the captured data, a surface model of the cartilage can be generated (and in some embodiments, a model for the bone structure can also be generated) in a manner similar to that used for the surface model and bone structure models populating the donor database. This surface model may subsequently be manipulated, e.g., rotated, sized, and the like, during the donor site identification procedure to compare the defect region to donor models in the donor database.

In some exemplary embodiments, the received data relating to the defect region of the patient can be used to identify data in the donor database corresponding to the patient's defect. In other words, instead of using the data relating to the defect region to identify a donor site by comparing the data of the defect region to the donor data in the database, the data relating to the defect region can be used to first identify a corresponding non-damaged cartilage structure, i.e., the counterpart healthy cartilage from the donor database which does not have a defect, which can subsequently be used to identify a suitable donor site to harvest bone-cartilage to repair the defect region.

With continued reference to FIG. 69, once the first data has been received and/or the data was used to generate a surface model to be used in identifying a suitable donor site or to first identify a corresponding healthy cartilage-bone counterpart from the donor database, at least one donor site from the donor database can be identified based on the first data relating to the defect region (704). Identifying the at least one donor site may include performing comparisons of the data representative of, for example, surface models of donor sites from the database to the first data relating to the defect region, or to some derivative data thereof (for example, a generated surface model for the defect region, a surface model of the same anatomical location but without the defect, or a relevant portion of whichever surface model is selected for performing the comparisons, e.g., only the area in the surface model which includes the defect region. Based on these comparisons, the at least one suitable donor site can be determined.

Further, in some exemplary embodiments, the surface model data obtained from the data relating to the defect region can be used to compare, for example, the dimensions and surface curvatures of the model, to the corresponding dimensions and curvature data of the plurality of donor sites in the donor database. The two surface models can be similarly scaled and/or directionally tagged to enable an accurate comparison. The dimensions and curvatures can thus be compared to determine if the particular cartilage would be a suitable donor site to harvest bone-cartilage to repair the defect region in the body of the patient.

In performing the comparisons to identify suitable donor sites, the model surfaces can be manipulated to place them in different orientations to facilitate the comparisons. In particular, the surface model corresponding to the defect region can be rotated relative to the donor site surface models to determine an optimal matching orientation for the models being compared. For example, the surface model of the defect region can be rotated to determine how the curvatures of the surface model match different areas of the surfaces model against which it is compared. Alternatively and/or additionally, in some embodiments, the donor site surface models can be manipulated, e.g., rotated, to compare how those surfaces match the surface model of the defect region in different spatial orientations. The manipulation of the surface models may be performed using the rendering application which was used to generate the surface model or by using a separate application which can perform the manipulation using the rendered models. The results of these comparisons may be expressed using, e.g., a matching score or metric representative of how well the two surfaces matched at the particular positions and/or orientations. The level of matching may be based on the extent to which the curvatures and dimensions of the surfaces being compared fit each other, i.e., to what extent the two surfaces are congruent to each other. Such a determination may be performed by, e.g., minimizing the difference between the topologies represented by the two surface models (such as finding min(Σ_{x,y,z}V_{defect(x,y,z)}−V_{donor(x,y,z)})), where V represents topology vector values by minimizing the least-square error of the difference between the surface model representations of the donor and defect sites. In some exemplary embodiments, the optimal matching position orientation of the model surfaces compared may be performed visually by the operator of the system, e.g., a surgeon, who examines the surfaces compared to each other and selects one position/orientation which appears to result in the best match, or by a processing device. The procedure of matching the model surface of the defect region to model surfaces of potential donor sites can be repeated for other sites.

To compare the surface model of a defect region to one or more donor site surface models through, e.g., computations based on topological features of the surfaces, and the like, the operations may be facilitated by overlaying the surface models against each other. The overlaying operations may be achieved by using built-in overlaying functions available on the particular graphical rendering application being implemented. For example, when using SolidWorks, the application's alignment function may be used to position two or more surface model appearing in a view against each other. Alternatively and/or additionally, custom-made procedures for aligning and/or overlaying multiple surface models may be implemented for use with the particular rendering application or independently of the particular rendering application.

As described herein, the donor database 614 may include donor sites from which irregularly-shaped bone-cartilage grafts, e.g., non-cylindrical grafts, can be harvested. Thus, in situations where the defect region has an irregular shape and the optimal shape of the graft would be one that is substantially similar to the irregular shape of the defect region, a surface model of the irregularly-shaped defect, generated in the manner described herein, can be used to identify suitable donor sites from which irregularly-shaped grafts can be harvested. Specifically, the surface model of such an irregularly-shaped defect region, which includes small surface segments representative of dimensions and curvatures defining the irregular shape, can be compared against one or more donor sites stored in the database 614 with respect to which similar dimension and curvature information is maintained. As described herein, such a comparison may be performed by computing, e.g., a minimum of the difference (or the least-square error) between the surface features of the surface models of the defect and surface features of the surface models of candidate donor sites. In performing such comparisons, the donor surface models and/or the surface model of defect region may be re-positioned and have their orientations manipulated to enable comparing surface features of the defect region against sub-areas in a particular donor site surface model. In other words, the matching of a defect region, e.g., an irregularly-shaped defect, includes, in some exemplary embodiments, not only identifying a suitable donor site, but also identifying appropriate sub-areas and orientations at the donor site.

In some exemplary embodiments, after identifying an appropriate position where the model surface of the defect region matches (or reasonably matches) the model surface of the donor site, a cross-sectional tool to obtain cross-sections of each surface relative to the other may be used. Such a cross-sectional tool may be implemented on the application used to render the models, e.g., Pro/Engineer, SolidWorks 3D, and the like, or by using another application, e.g., a software implemented tool. The cross-sections of each surface may be overlapped to determine congruence of, e.g., surface textures, contours, and the like.

To identify suitable donor sites, comparisons of the surface model corresponding to the defect region to surface models from the donor database may be performed according to a hierarchy of matching criteria. Thus, identified suitable donor sites may be ranked to provide a hierarchy of suitable sites from which a user, e.g., a surgeon, may select one or more of the listed sites. Examples of matching criteria include the dimensions and/or topological attributes of the donor sites, the defect directionality, the cartilage characteristics, the area around the defect, and the like. In some embodiments, evaluation of the quality of a particular suitable site may be performed in a manner analogous to the matching level score described above, in which the extent of how well the surface of the defect region matches the surface of a potential donor site is determined and a representative “topographical matching” score is generated. Another example of a matching criterion is the impact of the harvesting bone-cartilage from a particular donor site will have on the well-being of the individual. Particularly, harvesting bone-cartilage from one particular anatomical location may affect the mobility of the patient (in that the bone-cartilage may be used, under some circumstances, during movement of the patient), while harvesting bone-cartilage from another anatomical location may have little or no impact on the mobility of the patient (in that the bone-cartilage is not utilized for mobility). Accordingly, another score, i.e., an “impact” score, may be computed to represent the impact of harvesting bone-cartilage from a potential donor site. For example, various anatomical locations may be associated with predetermined impact values indicative of the impact harvesting bone-cartilage from the particular anatomical location would have on a patient's mobility or well-being. In some embodiments, a composite score which factors in the various scores derived for a particular anatomical location using the matching criteria may be determined Such a composite score may be computed, in some embodiments, as a weighted average of the various computed criteria scores for the anatomical location.

Thus, and with reference again to FIG. 69, having identified suitable donor sites from which bone-cartilage can be harvested, and, in some embodiments, having ranked those sites, one or more of the identified sites can be selected by, e.g., a surgeon (706). Optionally, templates to harvest bone-cartilage and/or remove damaged bone-cartilage may be generated (708). Alternatively, the templates used may be selected from a repertoire of standard, pre-generated templates. Generating custom templates may be based, at least in part, on the received data corresponding to the defect region and/or on the data corresponding to the identified donor sites (and/or their associated surface model data) from which bone-cartilage graft(s) to repair the defect can be harvested. In some embodiments, generating custom templates can be performed using a 3D printer, such as the 3D printer 616 depicted in FIG. 67. The surgeon may then proceed to perform the harvesting procedures at the selected sites by utilizing the instruments, systems and methods described above (710).

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. An instrument for capturing a surface topography of an anatomical location, comprising:
a plurality of elongated rod members, and
a locking mechanism for releasably securing the plurality of elongated rod members relative to each other,
wherein the plurality of elongated rod members are oriented to capture the surface topography of the anatomical location.

2. The instrument according to claim 1, wherein the plurality of elongated rod members are independently translatable relative to each other.

3. The instrument according to claim 1, wherein the surface topography of the anatomical location comprises a combination of a peripheral surface topography and a central surface topography of the anatomical location of a defect region.

4. The instrument according to claim 1, wherein the plurality of elongated rod members include a visual indicator thereon to indicate an effectiveness of the plurality of elongated rod members to capture the surface topography of the anatomical location.

5. An instrument for defining an implant region, comprising:
a template configured to be secured to an anatomical location, and
an adapter including a plurality of elongated rod members for capturing a surface topography surrounding a defect region.

6. The instrument according to claim 5, wherein the template defines a geometry configured and dimensioned to capture the defect region in the anatomical location.

7. The instrument according to claim 6, wherein the geometry of the template is one of a predetermined geometry or a variable geometry.

8. The instrument according to claim 7, wherein the predetermined geometry is one of an asymmetrical geometry or a symmetrical geometry.

9. The instrument according to claim 5, wherein the adapter is detachable from the template.

10. The instrument according to claim 5, comprising a driving mechanism for driving the template into the anatomical location and a cutter.

11. The instrument according to claim 10, wherein the driving mechanism is at least one of a hammer mechanism and a crank-actuated mechanism.

12. The instrument according to claim 11, wherein the hammer mechanism is a slap hammer and the crank-actuated mechanism includes a screw for anchoring the template to the defect region and an actuator for driving the template into the anatomical location.

13. (canceled)

14. The instrument according to claim 11, wherein as the driving mechanism drives the template into the anatomical location, the adapter captures the surface topography surrounding the defect region.

15. The instrument according to claim 10, wherein the cutter forms one of a smooth defect region cavity or a stepped defect region cavity.

16. The instrument according to claim 15, wherein the stepped defect region cavity creates a press fit between the stepped defect region cavity and a donor plug.

17. The instrument according to claim 5, wherein the plurality of elongated rod members include a visual indicator thereon to indicate an effectiveness of the plurality of elongated rod members to capture the surface topography surrounding the defect region.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. A graft harvesting device, comprising:
an elongated shaft, and
a cutting member mounted with respect to the elongated shaft,
wherein the cutting member is operative to form a harvest cavity of a predetermined geometry.

34. The device according to claim 33, comprising a plurality of elongated rod members for capturing a peripheral surface topography of an anatomical location surrounding the defect region cavity.

35. The device according to claim 34, comprising a locking mechanism for releasably locking the plurality of elongated rod members in a desired relative orientation.

36. The device according to claim 35, wherein the plurality of elongated rod members are actuated manually or electronically to translate against a surface of the anatomical location.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)
